SEQUENCE LISTING (28 sequences)

SEQ ID NO: 1 - DNA, 1755 bp, Pseudomonas mesoacidophila MX-45
atgcttatgaagagattattcgccgcgtctctgatgcttgctttttcaagcgtctcctctgtgagggctgaggaggccgtaaagccgggcgcgccatggtggaaaagtgctgtcttctatcaggtctatccgcgctcgttcaaggataccaacggtgatgggatcggcgatttcaaaggactgacggagaagctcgactatctcaaggggctcggcatagacgccatctggatcaatccacattacgcgtctcccaacaccgataatggctacgatatcagcgactatcgagaggtcatgaaggaatatgggacgatggaggacttcgatcgtctgatggctgagttgaagaagcgcggcatgcggctcatggttgatgtcgtgatcaaccattcgagtgaccaacacgaatggttcaagagcagccgggcctccaaagacaatccctaccgtgactattatttctggcgtgacggcaaagacggtcacgagccaaacaattacccttccttcttcggcggttcggcatgggagaaggaccccgtaaccgggcaatattacctgcattatttcggtcgtcagcagccagatctgaactgggacacgccgaagcttcgcgaggaactctatgcgatgctgcggttctggctcgacaagggcgtatcaggcatgcggttcgatacggtggctacctactcgaagacaccgggtttcccggatctgacaccggagcagatgaagaacttcgcggaggcctatacccaggggccgaaccttcatcgttacctgcaggaaatgcacgagaaggtcttcgatcattatgacgcggtcacggccggcgaaatcttcggcgctccgctcaatcaagtgccgctgttcatcgacagccggaggaaagagctggatatggctttcaccttcgatctgatccgttatgatcgcgcactggatcgttggcataccattccgcgtaccttagcggacttccgtcaaacgatcgataaggtcgacgccatcgcgggcgaatatggctggaacacgttcttcctcggcaatcacgacaatccccgtgcggtatcgcattttggtgacgatcggccgcaatggcgcgaagcctcggccaaggctctggccaccgtcaccttgacccagcgaggaacgccgttcatcttccaaggagatgaactcggaatgaccaactaccccttcaagacgctgcaggactttgatgatatcgaagtcaaaggcttctttcaggactatgtcgaaaccggaaaggcaactgccgaggaattgctgaccaatgtggcgttgactagccgcgacaacgcccgcacgccctttcaatgggatgacagtgctaatgcgggattcacgaccggcaagccttggctaaaggtcaatccaaactacactgagatcaacgccgcgcgggaaattggcgatcctaaatcggtctacagcttttaccgcaacctgatctcaatccggcatgaaactcccgctctttcgaccgggagctatcgcgacatcgatccgagtaatgccgatgtctatgcctatacgcgcagccaggatggcgagacctatctggtcgtagtcaacttcaaggcagagccaaggagtttcacgcttccggacggcatgcatattgccgaaaccctgattgagagcagttcgccagcagctccggcggcgggggctgcaagccttgagctgcagccttggcagtccggcatctacaaggtgaagtaa

SEQ ID NO: 2 - protein, 584 aa, Pseudomonas mesoacidophila MX-45
MLMKRLFAASLMLAFSSVSSVRAEEAVKPGAPWWKSAVFYQVYPRSFKDTNGDGIGDFKGLTEKLDYLKGLGIDAIWINPHYASPNTDNGYDISDYREVMKEYGTMEDFDRLMAELKKRGMRLMVDVVINHSSDQHEWFKSSRASKDNPYRDYYFWRDGKDGHEPNNYPSFFGGSAWEKDPVTGQYYLHYFGRQQPDLNWDTPKLREELYAMLRFWLDKGVSGMRFDTVATYSKTPGFPDLTPEQMKNFAEAYTQGPNLHRYLQEMHEKVFDHYDAVTAGEIFGAPLNQVPLFIDSRRKELDMAFTFDLIRYDRALDRWHTIPRTLADFRQTIDKVDAIAGEYGWNTFFLGNHDNPRAVSHFGDDRPQWREASAKALATVTLTQRGTPFIFQGDELGMTNYPFKTLQDFDDIEVKGFFQDYVETGKATAEELLTNVALTSRDNARTPFQWDDSANAGFTTGKPWLKVNPNYTEINAAREIGDPKSVYSFYRNLISIRHETPALSTGSYRDIDPSNADVYAYTRSQDGETYLVVVNFKAEPRSFTLPDGMHIAETLIESSSPAAPAAGAASLELQPWQSGIYKVK

SEQ ID NO: 3 - DNA, 1698 bp, Artificial Sequence
  MutB gene (derived from Pseudomonas mesoacidophila MX-45), N-terminal deleted, codon optimized
gctacaaatattcaaaagtccgcagactttccaatatggtggaaacaagctgtattttaccaaatatatccgagaagtttcaaagactctaatggcgatggcatcggggacatccccggtattatagagaagctcgactacttgaagatgctcggcgtcgatgccatctggattaatcctcactacgagagtcctaatacggacaacggctacgatatttcggactacaggaagataatgaaggaatacggttcaatggctgactttgatcggctcgtggctgagatgaacaagcgcggaatgaggttgatgatagacatagttattaaccacacctcggacagacatcggtggtttgtccaatctaggtcgggtaaggacaacccctaccgtgattactacttttggagggacggtaaacagggtcaagcgccgaacaactatccctcgttcttcggtggctccgcatggcaactcgacaaacaaacagatcagtattaccttcattactttgctccccagcaaccggatcttaactgggataacccaaaggtgcgcgctgaactctacgatattcttcgtttctggttggacaagggagtgtcgggcctccgttttgacaccgtcgccacattctcaaagatccccggatttcctgacctttcaaaggctcagcttaaaaacttcgctgaggcgtacaccgaagggccgaacatacataagtacatacacgagatgaaccgccaggtacttagtaagtataatgtcgcaactgcgggtgaaatattcggcgtccccgtcagcgccatgccagattactttgataggcggagggaggaactcaacatcgcgttcacctttgatttgatcaggcttgatcgctacccggatcagcgttggcgccgcaagccctggaccctgtctcagtttcggcaagtaatctcccagactgacagagcggcaggcgagttcggctggaatgccttctttttggataatcacgacaacccgaggcaagtcagtcactttggggatgacagcccacagtggcgtgaacgcagcgcgaaagcgctggctacactcttgcttactcagcgcgctaccccttttatattccaaggagcggagttggggatgacgaactatccattcaaaaatattgaagagttcgacgacattgaggttaagggcttctggaacgattacgttgcgtcaggaaaggtgaacgccgcagagttccttcaggaagtgcggatgaccagtagagacaactcgcgcacgcccatgcagtggaacgacagcgtgaacgctggctttacacaaggtaagccctggtttcacctcaaccctaattacaaacagataaacgctgccagggaggtgaacaagccagattcggtcttttcctattaccgccagcttatcaatcttcggcaccagattccggctctgacatctggcgagtacagggacctggacccacaaaataatcaagtttatgcttatactcgcattttggacaacgagaagtatctcgtcgttgtgaactttaaaccagagcaacttcattatgctcttccggacaatttgacaatagcaagcagcttgctcgaaaatgtacatcagccctccctccaagaaaatgctagtacactgactctcgctccgtggcaggctggaatatataagctgaactga

SEQ ID NO: 4 - protein, 565 aa, Artificial Sequence
  MutB polypeptide (derived from Pseudomonas mesoacidophila MX-45), N-terminal deleted, codon optimized
ATNIQKSADFPIWWKQAVFYQIYPRSFKDSNGDGIGDIPGIIEKLDYLKMLGVDAIWINPHYESPNTDNGYDISDYRKIMKEYGSMADFDRLVAEMNKRGMRLMIDIVINHTSDRHRWFVQSRSGKDNPYRDYYFWRDGKQGQAPNNYPSFFGGSAWQLDKQTDQYYLHYFAPQQPDLNWDNPKVRAELYDILRFWLDKGVSGLRFDTVATFSKIPGFPDLSKAQLKNFAEAYTEGPNIHKYIHEMNRQVLSKYNVATAGEIFGVPVSAMPDYFDRRREELNIAFTFDLIRLDRYPDQRWRRKPWTLSQFRQVISQTDRAAGEFGWNAFFLDNHDNPRQVSHFGDDSPQWRERSAKALATLLLTQRATPFIFQGAELGMTNYPFKNIEEFDDIEVKGFWNDYVASGKVNAAEFLQEVRMTSRDNSRTPMQWNDSVNAGFTQGKPWFHLNPNYKQINAAREVNKPDSVFSYYRQLINLRHQIPALTSGEYRDLDPQNNQVYAYTRILDNEKYLVVVNFKPEQLHYALPDNLTIASSLLENVHQPSLQENASTLTLAPWQAGIYKLN

SEQ ID NO: 5 - DNA, 1800 bp, Artificial Sequence
  MutB gene (derived from Pseudomonas mesoacidophila MX-45), N-terminal deleted, plus nucleic acid encoding ER signal peptide and N′ terminal propeptide (NTPP) from sweet potato sporamin, codon optimized
atgaaagcctttacactcgcattgttcctcgcattgtctctgtatttgctgccgaacccggcgcattctaggttcaacccgataagactccccaccacccatgaaccggccgccgaagaggcggtcaagcccggtgccccctggtggaagtcagcagtgttttaccaggtctatccacgctcattcaaggatacgaacggggatggcattggcgattttaagggcctcacagaaaaactcgactaccttaaggggctcgggattgacgctatctggatcaacccgcattacgcgagtcctaatactgacaatggatatgatatttcagattatcgcgaagttatgaaggaatacggaactatggaggatttcgatcggcttatggcagagctgaaaaagaggggcatgcgtctgatggtggacgtggttatcaatcacagtagcgatcagcacgagtggttcaagtccagtagggcctctaaggataatccctacagggactattacttttggcgcgacggaaaggacggccacgagcctaacaactacccatcgttctttgggggaagcgcttgggagaaagatccagtcacgggtcagtactacttgcactatttcgggcgtcagcaaccggatttgaattgggacacaccaaagctccgcgaggagttgtatgccatgttgcgcttctggcttgataagggtgttagtgggatgagattcgatacagttgccacatatagtaagactccgggcttcccggatctcacccccgagcagatgaagaactttgcggaagcctacacccagggcccaaacttgcaccggtatctgcaagagatgcacgaaaaggtgtttgatcactacgatgccgtgaccgccggtgagattttcggagcccctctgaaccaggtgccgctcttcattgatagccgccgcaaggagcttgacatggcgttcactttcgatctcatacggtacgaccgggcgcttgaccgttggcacaccatcccaagaacattggcggatttcagacagaccatcgacaaagtggatgccattgccggagaatacggctggaatacctttttcctcggtaaccacgataaccctcgtgcagtttcccactttggagatgatcgtccccagtggcgtgaggcatccgccaaggctcttgcgaccgtgacgctcacacagaggggcactccattcatctttcaaggtgatgagctgggcatgaccaattacccgtttaagactcttcaggacttcgacgacatcgaggtcaaggggttcttccaggactatgtggagacaggcaaggcaaccgccgaggagctgctcacaaacgttgcgttgacgtctcgggataacgcgcggaccccattccagtgggacgacagtgctaatgcaggcttcaccacggggaagccttggctgaaggtgaatccgaattatactgagatcaatgcagcgcgcgaaattggagatcctaagagcgtgtactccttctaccgcaacctgataagcattcgtcacgaaactcccgccctctcaaccgggtcatatagagacatcgaccctagcaacgctgacgtctacgcttacacccgcagtcaagatggtgagacttacctcgtcgtcgttaacttcaaggctgaacccaggtcattcactcttccggatggcatgcacatagccgaaaccctgatcgagtcttcctctccagcggccccagcggctggtgccgcgagcctcgaactccaaccatggcagtccgggatatataaggtgaagtaa

SEQ ID NO: 6 - protein, 599 aa, Artificial Sequence
  MutB polypeptide (derived from Pseudomonas mesoacidophila MX-45), N-terminal deleted, plus ER signal peptide and N′ terminal propeptide (NTPP) from sweet potato sporamin, codon optimized
MKAFTLALFLALSLYLLPNPAHSRFNPIRLPTTHEPAAEEAVKPGAPWWKSAVFYQVYPRSFKDTNGDGIGDFKGLTEKLDYLKGLGIDAIWINPHYASPNTDNGYDISDYREVMKEYGTMEDFDRLMAELKKRGMRLMVDVVINHSSDQHEWFKSSRASKDNPYRDYYFWRDGKDGHEPNNYPSFFGGSAWEKDPVTGQYYLHYFGRQQPDLNWDTPKLREELYAMLRFWLDKGVSGMRFDTVATYSKTPGFPDLTPEQMKNFAEAYTQGPNLHRYLQEMHEKVFDHYDAVTAGEIFGAPLNQVPLFIDSRRKELDMAFTFDLIRYDRALDRWHTIPRTLADFRQTIDKVDAIAGEYGWNTFFLGNHDNPRAVSHFGDDRPQWREASAKALATVTLTQRGTPFIFQGDELGMTNYPFKTLQDFDDIEVKGFFQDYVETGKATAEELLTNVALTSRDNARTPFQWDDSANAGFTTGKPWLKVNPNYTEINAAREIGDPKSVYSFYRNLISIRHETPALSTGSYRDIDPSNADVYAYTRSQDGETYLVVVNFKAEPRSFTLPDGMHIAETLIESSSPAAPAAGAASLELQPWQSGIYKVK

SEQ ID NO: 7 - DNA, 1797 bp, Pantoea dispersa
atgtttcttaatggatttaagacagttattgctctgactatggcaagctcgttttatcttgccgccagcccgttaactaagccatcgacccctattgccgcaacgaatatacaaaagtccgctgattttcccatttggtggaaacaggcagtattttaccagatttatccccgctcatttaaagatagcaatggtgatggtatcggcgatattcccggtatcattgagaaactggactatttaaaaatgctgggagttgatgctatctggataaacccgcactatgagtctcctaacaccgacaatggttacgatattagtgattatcgtaaaatcatgaaggagtacggcagcatggctgactttgaccgtctggttgccgaaatgaataaacgtggtatgcgcctgatgattgatattgttatcaatcataccagcgatcgtcaccgctggtttgtgcagagccgttcaggtaaagataatccttaccgcgactattatttctggcgtgatggtaaacagggacaggctcccaataactatccctctttctttggcggttcagcctggcaactggataaacagactgaccagtattatctgcactattttgcaccacagcagccggatctgaactgggataacccaaaagttcgggctgaactctacgatattctgcgtttctggctggataaaggcgtatccggactacgttttgataccgtggctactttctccaaaattcctggcttcccggacctgtcaaaagcgcagctgaagaattttgccgaagcttatactgaggggccgaatattcataaatatatccatgaaatgaaccgccaggtactgtctaaatataatgttgccaccgctggtgaaatcttcggtgtgccagtgagtgctatgccggattattttgaccggcggcgtgaagaactcaatattgctttcacctttgatttgatcaggctcgatcgttatcccgatcagcgctggcgtcgtaaaccatggacattaagccagtttcgtcaagttatctctcagactgaccgtgccgccggtgaatttggctggaacgcctttttccttgataaccatgataacccgcgccaggtctcacactttggtgacgacagcccacaatggcgcgaacgctcggcaaaagcactggcaacgctgctgctgacgcagcgtgccacgccgtttatctttcagggggcggagttgggaatgactaattacccctttaaaaatatagaggaatttgatgatattgaggttaaaggcttctggaacgactatgtagccagcggaaaagtaaacgctgctgaatttttacaggaggttcgcatgaccagccgcgataacagccgaacaccaatgcagtggaacgactctgttaatgccggattcacccagggcaaaccctggtttcacctcaatcccaactataagcaaatcaatgccgccagggaggtgaataaacccgactcggtattcagttactaccgtcaactgatcaacctgcgtcaccagatcccggcactgaccagtggtgaataccgtgatctcgatccgcagaataaccaggtctatgcctatacccgtatactggataatgaaaaatatctggtggtagttaattttaaacctgagcagctgcattacgctctgccagataatctgactattgccagcagtctgctggaaaatgtccaccaaccatcactgcaagaaaatgcctccacgctgactcttgctccgtggcaagccgggatctataagctgaactga

SEQ ID NO: 8 - protein, 598 aa, Pantoea dispersa
MFLNGFKTVIALTMASSFYLAASPLTKPSTPIAATNIQKSADFPIWWKQAVFYQIYPRSFKDSNGDGIGDIPGIIEKLDYLKMLGVDAIWINPHYESPNTDNGYDISDYRKIMKEYGSMADFDRLVAEMNKRGMRLMIDIVINHTSDRHRWFVQSRSGKDNPYRDYYFWRDGKQGQAPNNYPSFFGGSAWQLDKQTDQYYLHYFAPQQPDLNWDNPKVRAELYDILRFWLDKGVSGLRFDTVATFSKIPGFPDLSKAQLKNFAEAYTEGPNIHKYIHEMNRQVLSKYNVATAGEIFGVPVSAMPDYFDRRREELNIAFTFDLIRLDRYPDQRWRRKPWTLSQFRQVISQTDRAAGEFGWNAFFLDNHDNPRQVSHFGDDSPQWRERSAKALATLLLTQRATPFIFQGAELGMTNYPFKNIEEFDDIEVKGFWNDYVASGKVNAAEFLQEVRMTSRDNSRTPMQWNDSVNAGFTQGKPWFHLNPNYKQINAAREVNKPDSVFSYYRQLINLRHQIPALTSGEYRDLDPQNNQVYAYTRILDNEKYLVVVNFKPEQLHYALPDNLTIASSLLENVHQPSLQENASTLTLAPWQAGIYKLN

SEQ ID NO: 9 - DNA, 1698 bp, Artificial Sequence; identical to SEQ ID NO: 3
  UQ68J gene (derived from Pantoea dispersa), N-terminal deleted, codon optimized

SEQ ID NO: 10 - protein, 565 aa, Artificial Sequence; identical to SEQ ID NO: 4
  UQ68J polypeptide (derived from Pantoea dispersa), N-terminal deleted, codon optimized

SEQ ID NO: 11 - DNA, 1809 bp, Artificial Sequence
  UQ68J gene (derived from Pantoea dispersa), N-terminal deleted, plus nucleic acid encoding ER signal peptide and N′ terminal propeptide (NTPP) from sweet potato sporamin, codon optimized
atgaaagcctttacactcgcattgttcctcgcattgtctctgtatttgctgccgaacccggcgcattctaggttcaacccgataagactccccaccacccatgaaccggccgctacaaatattcaaaagtccgcagactttccaatatggtggaaacaagctgtattttaccaaatatatccgagaagtttcaaagactctaatggcgatggcatcggggacatccccggtattatagagaagctcgactacttgaagatgctcggcgtcgatgccatctggattaatcctcactacgagagtcctaatacggacaacggctacgatatttcggactacaggaagataatgaaggaatacggttcaatggctgactttgatcggctcgtggctgagatgaacaagcgcggaatgaggttgatgatagacatagttattaaccacacctcggacagacatcggtggtttgtccaatctaggtcgggtaaggacaacccctaccgtgattactacttttggagggacggtaaacagggtcaagcgccgaacaactatccctcgttcttcggtggctccgcatggcaactcgacaaacaaacagatcagtattaccttcattactttgctccccagcaaccggatcttaactgggataacccaaaggtgcgcgctgaactctacgatattcttcgtttctggttggacaagggagtgtcgggcctccgttttgacaccgtcgccacattctcaaagatccccggatttcctgacctttcaaaggctcagcttaaaaacttcgctgaggcgtacaccgaagggccgaacatacataagtacatacacgagatgaaccgccaggtacttagtaagtataatgtcgcaactgcgggtgaaatattcggcgtccccgtcagcgccatgccagattactttgataggcggagggaggaactcaacatcgcgttcacctttgatttgatcaggcttgatcgctacccggatcagcgttggcgccgcaagccctggaccctgtctcagtttcggcaagtaatctcccagactgacagagcggcaggcgagttcggctggaatgccttctttttggataatcacgacaacccgaggcaagtcagtcactttggggatgacagcccacagtggcgtgaacgcagcgcgaaagcgctggctacactcttgcttactcagcgcgctaccccttttatattccaaggagcggagttggggatgacgaactatccattcaaaaatattgaagagttcgacgacattgaggttaagggcttctggaacgattacgttgcgtcaggaaaggtgaacgccgcagagttccttcaggaagtgcggatgaccagtagagacaactcgcgcacgcccatgcagtggaacgacagcgtgaacgctggctttacacaaggtaagccctggtttcacctcaaccctaattacaaacagataaacgctgccagggaggtgaacaagccagattcggtcttttcctattaccgccagcttatcaatcttcggcaccagattccggctctgacatctggcgagtacagggacctggacccacaaaataatcaagtttatgcttatactcgcattttggacaacgagaagtatctcgtcgttgtgaactttaaaccagagcaacttcattatgctcttccggacaatttgacaatagcaagcagcttgctcgaaaatgtacatcagccctccctccaagaaaatgctagtacactgactctcgctccgtggcaggctggaatatataagctgaactga

SEQ ID NO: 12 - protein, 602 aa, Artificial Sequence
  UQ68J polypeptide (derived from Pantoea dispersa), N-terminal deleted, plus ER signal peptide and N′ terminal propeptide (NTPP) from sweet potato sporamin,  codon optimized
MKAFTLALFLALSLYLLPNPAHSRFNPIRLPTTHEPAATNIQKSADFPIWWKQAVFYQIYPRSFKDSNGDGIGDIPGIIEKLDYLKMLGVDAIWINPHYESPNTDNGYDISDYRKIMKEYGSMADFDRLVAEMNKRGMRLMIDIVINHTSDRHRWFVQSRSGKDNPYRDYYFWRDGKQGQAPNNYPSFFGGSAWQLDKQTDQYYLHYFAPQQPDLNWDNPKVRAELYDILRFWLDKGVSGLRFDTVATFSKIPGFPDLSKAQLKNFAEAYTEGPNIHKYIHEMNRQVLSKYNVATAGEIFGVPVSAMPDYFDRRREELNIAFTFDLIRLDRYPDQRWRRKPWTLSQFRQVISQTDRAAGEFGWNAFFLDNHDNPRQVSHFGDDSPQWRERSAKALATLLLTQRATPFIFQGAELGMTNYPFKNIEEFDDIEVKGFWNDYVASGKVNAAEFLQEVRMTSRDNSRTPMQWNDSVNAGFTQGKPWFHLNPNYKQINAAREVNKPDSVFSYYRQLINLRHQIPALTSGEYRDLDPQNNQVYAYTRILDNEKYLVVVNFKPEQLHYALPDNLTIASSLLENVHQPSLQENASTLTLAPWQAGIYKLN

SEQ ID NO: 13 - protein, 598 aa, Artificial Sequence
  Modified UQ68J protein
MFLNGFKTVIALTMASSFYLAASPLTKPSTPIAATNIQKSADFPIWWKQAVFYQIYPRSFKDSNGDGIGDIPGIIEKLDYLKMLGVDAIWINPHYESPNTDNGYDISDYRKIMKEYGSMADFDRLVAEMNKRGMRLMIDIVINHTSDRHRWFVQSRSGRNNPYRDYYFWRDGRNGNAPNNYPSFFGGSAWQLDKQTDQYYLHYFAPQQPDLNWDNPKVRADLYAILRFWLDKGVSGLRFDTVATFSKIPGFPNLTPEQLKNFAEAYTEGPNIHKYIHEMNRQVLSKYNVATAGEIFGVPVDAMPQYFDRRREELNIAFTFDLIRLDRDSNERWRRKPWTLSQFRQVISQTDKAAGKYGWNAFFLDNHDNPRQVSHFGDDSPQWRERSAKALATLLLTQRATPFIFQGAELGMTNYPFRQLNEFDDIEVKGFWNDYVASGKVNAAEFLQEVRMTSRDNSRTPMQWNDSVNAGFTQGKPWFHLNPNYKQINAAREVTREDSVFSYYKKMINLRHQIPALTSGEYRDLDPQNNQVYAYTRILDNEKYLVVVNFKPEQLHYALPDNLTIASSLLENVHQPSLQENASTLTLAPWQAGIYKLN

SEQ ID NO: 14 - DNA, 1698 bp, Artificial Sequence
  Modified UQ68J gene, N-terminal deleted, codon optimized
gctacaaatattcaaaagtccgcagactttccaatatggtggaaacaagctgtattttaccaaatatatccgagaagtttcaaagactctaatggcgatggcatcggggacatccccggtattatagagaagctcgactacttgaagatgctcggcgtcgatgccatctggattaatcctcactacgagagtcctaatacggacaacggctacgatatttcggactacaggaagataatgaaggaatacggttcaatggctgactttgatcggctcgtggctgagatgaacaagcgcggaatgaggttgatgatagacatagttattaaccacacctcggacagacatcggtggtttgtccaatctaggtcgggtcgcaacaacccctaccgtgattactacttttggagggacggtcgcaacggtaacgcgccgaacaactatccctcgttcttcggtggctccgcatggcaactcgacaaacaaacagatcagtattaccttcattactttgctccccagcaaccggatcttaactgggataacccaaaggtgcgcgctgacctctacgccattcttcgtttctggttggacaagggagtgtcgggcctccgttttgacaccgtcgccacattctcaaagatccccggatttcctaaccttaccccagagcagcttaaaaacttcgctgaggcgtacaccgaagggccgaacatacataagtacatacacgagatgaaccgccaggtacttagtaagtataatgtcgcaactgcgggtgaaatattcggcgtccccgtcgacgccatgccacaatactttgataggcggagggaggaactcaacatcgcgttcacctttgatttgatcaggcttgatcgcgactccaacgagcgttggcgccgcaagccctggaccctgtctcagtttcggcaagtaatctcccagactgacaaagcggcaggcaagtacggctggaatgccttctttttggataatcacgacaacccgaggcaagtcagtcactttggggatgacagcccacagtggcgtgaacgcagcgcgaaagcgctggctacactcttgcttactcagcgcgctaccccttttatattccaaggagcggagttggggatgacgaactatccattccgccaactcaacgagttcgacgacattgaggttaagggcttctggaacgattacgttgcgtcaggaaaggtgaacgccgcagagttccttcaggaagtgcggatgaccagtagagacaactcgcgcacgcccatgcagtggaacgacagcgtgaacgctggctttacacaaggtaagccctggtttcacctcaaccctaattacaaacagataaacgctgccagggaggtgacccgcgaagattcggtcttttcctattacaagaagatgatcaatcttcggcaccagattccggctctgacatctggcgagtacagggacctggacccacaaaataatcaagtttatgcttatactcgcattttggacaacgagaagtatctcgtcgttgtgaactttaaaccagagcaacttcattatgctcttccggacaatttgacaatagcaagcagcttgctcgaaaatgtacatcagccctccctccaagaaaatgctagtacactgactctcgctccgtggcaggctggaatatataagctgaactga

SEQ ID NO: 15 - protein, 565 aa, Artificial Sequence
  Modified UQ68J polypeptide, N-terminal deleted, codon optimized
ATNIQKSADFPIWWKQAVFYQIYPRSFKDSNGDGIGDIPGIIEKLDYLKMLGVDAIWINPHYESPNTDNGYDISDYRKIMKEYGSMADFDRLVAEMNKRGMRLMIDIVINHTSDRHRWFVQSRSGRNNPYRDYYFWRDGRNGNAPNNYPSFFGGSAWQLDKQTDQYYLHYFAPQQPDLNWDNPKVRADLYAILRFWLDKGVSGLRFDTVATFSKIPGFPNLTPEQLKNFAEAYTEGPNIHKYIHEMNRQVLSKYNVATAGEIFGVPVDAMPQYFDRRREELNIAFTFDLIRLDRDSNERWRRKPWTLSQFRQVISQTDKAAGKYGWNAFFLDNHDNPRQVSHFGDDSPQWRERSAKALATLLLTQRATPFIFQGAELGMTNYPFRQLNEFDDIEVKGFWNDYVASGKVNAAEFLQEVRMTSRDNSRTPMQWNDSVNAGFTQGKPWFHLNPNYKQINAAREVTREDSVFSYYKKMINLRHQIPALTSGEYRDLDPQNNQVYAYTRILDNEKYLVVVNFKPEQLHYALPDNLTIASSLLENVHQPSLQENASTLTLAPWQAGIYKLN

SEQ ID NO: 16 - DNA, 1809 bp, Artificial Sequence
  Modified UQ68J gene, N-terminal deleted, plus nucleic acid encoding ER signal peptide and N′ terminal propeptide (NTPP) from sweet potato sporamin, codon optimized
atgaaggccttcaccctcgccctcttcctcgccctctccctctacctcctcccgaacccggcccactcccgcttcaacccgatccgcctcccgaccacccacgagccggccgctacaaatattcaaaagtccgcagactttccaatatggtggaaacaagctgtattttaccaaatatatccgagaagtttcaaagactctaatggcgatggcatcggggacatccccggtattatagagaagctcgactacttgaagatgctcggcgtcgatgccatctggattaatcctcactacgagagtcctaatacggacaacggctacgatatttcggactacaggaagataatgaaggaatacggttcaatggctgactttgatcggctcgtggctgagatgaacaagcgcggaatgaggttgatgatagacatagttattaaccacacctcggacagacatcggtggtttgtccaatctaggtcgggtcgcaacaacccctaccgtgattactacttttggagggacggtcgcaacggtaacgcgccgaacaactatccctcgttcttcggtggctccgcatggcaactcgacaaacaaacagatcagtattaccttcattactttgctccccagcaaccggatcttaactgggataacccaaaggtgcgcgctgacctctacgccattcttcgtttctggttggacaagggagtgtcgggcctccgttttgacaccgtcgccacattctcaaagatccccggatttcctaaccttaccccagagcagcttaaaaacttcgctgaggcgtacaccgaagggccgaacatacataagtacatacacgagatgaaccgccaggtacttagtaagtataatgtcgcaactgcgggtgaaatattcggcgtccccgtcgacgccatgccacaatactttgataggcggagggaggaactcaacatcgcgttcacctttgatttgatcaggcttgatcgcgactccaacgagcgttggcgccgcaagccctggaccctgtctcagtttcggcaagtaatctcccagactgacaaagcggcaggcaagtacggctggaatgccttctttttggataatcacgacaacccgaggcaagtcagtcactttggggatgacagcccacagtggcgtgaacgcagcgcgaaagcgctggctacactcttgcttactcagcgcgctaccccttttatattccaaggagcggagttggggatgacgaactatccattccgccaactcaacgagttcgacgacattgaggttaagggcttctggaacgattacgttgcgtcaggaaaggtgaacgccgcagagttccttcaggaagtgcggatgaccagtagagacaactcgcgcacgcccatgcagtggaacgacagcgtgaacgctggctttacacaaggtaagccctggtttcacctcaaccctaattacaaacagataaacgctgccagggaggtgacccgcgaagattcggtcttttcctattacaagaagatgatcaatcttcggcaccagattccggctctgacatctggcgagtacagggacctggacccacaaaataatcaagtttatgcttatactcgcattttggacaacgagaagtatctcgtcgttgtgaactttaaaccagagcaacttcattatgctcttccggacaatttgacaatagcaagcagcttgctcgaaaatgtacatcagccctccctccaagaaaatgctagtacactgactctcgctccgtggcaggctggaatatataagctgaactga

SEQ ID NO: 17 - protein, 602 aa, Artificial Sequence
  Modified UQ68J polypeptide, N-terminal deleted, plus N′ terminal propeptide (NTPP) from sweet potato sporamin, codon optimized
MKAFTLALFLALSLYLLPNPAHSRFNPIRLPTTHEPAATNIQKSADFPIWWKQAVFYQIYPRSFKDSNGDGIGDIPGIIEKLDYLKMLGVDAIWINPHYESPNTDNGYDISDYRKIMKEYGSMADFDRLVAEMNKRGMRLMIDIVINHTSDRHRWFVQSRSGRNNPYRDYYFWRDGRNGNAPNNYPSFFGGSAWQLDKQTDQYYLHYFAPQQPDLNWDNPKVRADLYAILRFWLDKGVSGLRFDTVATFSKIPGFPNLTPEQLKNFAEAYTEGPNIHKYIHEMNRQVLSKYNVATAGEIFGVPVDAMPQYFDRRREELNIAFTFDLIRLDRDSNERWRRKPWTLSQFRQVISQTDKAAGKYGWNAFFLDNHDNPRQVSHFGDDSPQWRERSAKALATLLLTQRATPFIFQGAELGMTNYPFRQLNEFDDIEVKGFWNDYVASGKVNAAEFLQEVRMTSRDNSRTPMQWNDSVNAGFTQGKPWFHLNPNYKQINAAREVTREDSVFSYYKKMINLRHQIPALTSGEYRDLDPQNNQVYAYTRILDNEKYLVVVNFKPEQLHYALPDNLTIASSLLENVHQPSLQENASTLTLAPWQAGIYKLN

SEQ ID NO: 18 - DNA, 63 bp, Artificial Sequence
  Nucleic Acid encoding ER signal peptide, codon optimized
atgaaggccttcaccctcgccctcttcctcgccctctccctctacctcctcccgaacccggcc

SEQ ID NO: 19 - protein, 21 aa, Artificial Sequence
  ER signal peptide
MKAFTLALFLALSLYLLPNPA

SEQ ID NO: 20 - DNA, 48 bp, Artificial Sequence
  Nucleic Acid encoding N′ terminal propeptide (NTPP) from sweet potato sporamin, codon optimized
cactcccgcttcaacccgatccgcctcccgaccacccacgagccggcc

SEQ ID NO: 21 - protein, 16 aa, Artificial Sequence
  N′ terminal propeptide (NTPP) from sweet potato sporamin
HSRFNPIRLPTTHEPA

SEQ ID NO: 22 - DNA, 2164 bp, Oryza sativa
atatcttgtttgatcccacctatctttggcattgacagatcattcaccattgaattgattgacagcatagctgagtctgcagagttcagttctggtaatgcccataaattttgaaggtgcggaaggaaaatgagggcagaagataggaaataccattaaagttgcattcacgtaatattcgaagcacagctgaaaattacttgccttatatcgtgacatgtagggaatggatctttctgtgttactccctttggggcaatgttacttcttacatgaataatgcttgacctttttctaaaatactgtgttttgaccttgacattctgtttgagatctatatagaatgcttttaagactcttttttgaagcattatgacccttgtggttcactgttttccttttagtttctgtttgctacaaagtgactcattgttggttgctactgtaagttgtgaacataaacaacagaattgatggctcatggctagatattatatcatacataaagaaacatagcaccataggcatctagatcatgctgcagatgcaagtaaggtgctcctgttaataagttggcactagttctcaatcaaccaaaataattaccgaacacatgcaaaccaagttataatcctgttccttttcttcttttttttttgagaaaaatactttcagagttgggcaaatgtaggtagtcaaacatggtccctgcttggtcaagaatgattctgggataatgtgctctcctaatgtatctaaagccagctccagatttgttactgctcaatggccagtgcatgttatgtctggtcgatgaaaacaattgatcctctattggctaggatacagttctaataagctgctacttgatagccagctattcaggcacaaagtggttgaaccatccagttttcattggtttctaacaaaactagatagaatatgtgattcttgatattgcttgttcttcacaaaattgacgggggatggcatattatgaccaatggtacccaaccaattggtattggcgggaccttgaatcttgactggtatatgatgttactgattgggtgcatttctgagagggtattcactatctccatgtcagccacctattaaaaaaaagtcaaaagttcagaaattttcataggagtgttcgacttgtttgtgcccagcaactaaatcccacttcggtgaagcggttgaaccccacaatcggtgctagtgatttcctccctgtacaagtggaaaattccatcctaaaatacagttagttctttttctgttttgtttagttatatgcatataaggatgcctccgttgggctcaagagctctagtcgtacatgaaactgagatcttatcgaatttaagtgcatgttatggaagctgatttatttagagggagggacttctgactttgtcatccaggatttggaaatgtaatgctttggataaacatgagttagtggctaaacaggagtgtcacattgttctaactccacactcttaaaggcatagctctaaaggtggagagtgctccttcagctttttgtaatgggtgcctttcttttttgccccctctcttgctgatagtagattcagtcacacatcatgagtttttgaagttcgttttgttggtgagggcttaagtacaaggttggtgattaacttgctgtatctacagttctacagaattttctttcttttattagaaacacagatataggttgttcattaaatgtcaatgaactaatagtgaaaatcaaggcagatatagttagatgaagtgtgcacactgcaaaagagatgcacagattttagaagtatataggtttttcaggaaaaaaaacaatttacaagtatatattccctgggaactggaactggatctttgtacatcttcttgttatataatataggtctataattgttctgttaagtaaaatactctggatagtattgggtttgtaagtggaatgagaatttctctagagaaggaatatagttagaattgacatgatgagggaagaacaacctcagtgtgaagtccagcaaatgagcacttggcacagtttttactttcattgtcctttctccactaagcatgctgcgctgtccaatttaaaatgctaccatgcagattctgggttctttgcaattga

SEQ ID NO: 23 - DNA, 3006 bp, Saccharum spp.
gcataggcattgtaaaagcggtatgcctcttcttcagtgcagaatttcataccaaccttaggtatcctgtcttccatagaattttctacctgagtaggttcggtctggttggatttgtagcgggtttcatgcaaaataagttagaaatcgtgcaaacttgcaatggaggttaaatttgaaatatatttgcatagacaaaacaaatatagattatgaatggtaatccaatatgacttgcattttctaactctattgctactgtgccagatgaagaatgttgatctggagaagttttgtgagaatgtgacaacaacgggaggtcatatcaagattctgggtacccgcggagaatcggcctccatgtagttagcctcgtcaggcatggggggaattggctgagatgcccccatgtagtcgtcaggcatggagagtactggctgagatgccattgttgtgtagatcgagagaaacgagaagaatgctagtctaataatacccttccgtatgctaaccaactattataattggcaccatttttcacatgctagcgccttttgcctgctttatttaattcaattgggtccgataagcatgtgaacgtgggagacggttccgtcggacggctccgttttcttgtagcgtacggcgtggacggagaaaaggtgagggcctatctctaaaggggaacgaatggatggtggacacgtgtggggagacaccgaagggacatgccgaggaggcacacaagcttcagcaggcgtctccagactctcagaagaagaagaagctcacggcacggttgcggctggttcttgctgtcgctgtctcgtggtgcacgtttctgtgatcacgctgaaatcgaccggccggcggaccaacaggaggtcagctcggccactccgtctccgagcgcatgagtgcaccgttcgtccgcggttccttttctcgtggtgccgtgcacgcctctgcgttcaccggcaccctgaaaccaatcagaacgttccctttacaggggaaagggacaagtctgataacctctctgtttccatcgtcctctaaccgcgaagagcggacgcacaagacttagagtctatttgttcgaaattttttactctcacaaaagctagcttttatagacgggcataaaagctatcatgtcgaccggcacgtttaatatttaacttataccatatgaatatcatgtcgaactatgaggatgatacttttctgaacgtgattgcgtgagttattaaattgtacttttagttgtttgagcatgaaggtctgaactatgaatttatgatgtattgtggcttgtgagctactccgctctacatttagttggtatcataaatattattatattatcatataaatttgatcaacttgagatgctttgactcttcaagattcttggaatgacttatcatttggggtagggagtaggtttctaaggccagtctcagtggggtttcatcagagtttcatggacattaaataagctgatgtgacaccgtattgatgaagagagagatgataagagtttcatgcgagtagagagagtttcatggggatgaaactcttcttcactgtttccaaaatatagatgcattggtaagagggccatgaaatctctagtgacactgacctaagatgagattgactctagcactatgtttcaaaatctgcatgcatgcatgctttgaatattgtaacctcacattaactcccctcacacatgcatgcaaacgggcggtgcacgcaaaagaattgagtgaagatgcacatgaaaaataagtaaaatgctttggcttcatcacccggcttaaatgctcgacagaaaaacacgtcggtagtcaaggttgtgcctaacaaactggggttcacatgtaaaacacgttcatgccttagaaacggcctggagggattagatacaacttcaattatatcttagggcccctccaatattgtcagctctaaactagttttatgtgtcacggtggaggagagggaggctaaaaatataatcttgagctaacgtgaagagaagagctatttttttttgctccccaatacatgatagatacaatatgagagaaaaaatatatgaataaagaacactttacatgccagccatacaatatgagatttcatctaagagccaacacctgactcgtactgttgaaggtgtcctagttggagtggtcgatcttttagttgttagtagtgtaagacctagtttagtgctcttttcttgtctaggtttatgttgtgttttggctgccaagtgttgaacaactcaaggtaaggtcccatctaattctaaaatgatgccaaataaagatagattacaaagttaaacgacggaaaaactctaaaataggatggaaagttttgtagagtaataattggtatgaagtggcgaagtcgaccacaaccaaacataaagagttaaatgcatggtaggctcttgatcttgtctggaggtgccacttaggtccacaaactctcaaattgcatttttgacaccctaatgttattcaagtgtgccacttagatctacaaactctcaaaatgcatttctgataccctagtgttgttcaagtgtgtcacttaggcaagaaaagttagataattttgataagctatgggaccaaattaatttatgtatgcatgctcgaactagttgatgatgatggaccccataatagacactagttcatgggctggtttccttgtatagtactagctagtataactttttcaagttgtagctactactttagcttatactccgcatattacaatcaaatagaattcggaagtactataaacgggagcctataaatggagacgttttgcatcatgaggctataacaacttgagcaaaaacagaagccgtgcgccatggcgctcagcaaggagcaacacacaagcactgatcagcaggccgtgctggatgctcagctccagctctggcaccacaccctgggctatgtcaagtcc

SEQ ID NO: 24 - DNA, 2686 bp, Saccharum spp.
tctagataatacgactcactatagggcacgcgtggtcgacggcccgggctggtctgcgacagctagaggcgccaccgcgtcctagcttcctccaacttctcgtcggagatcccttcagggatgcccaatgccaccgcccctaagtcaacctgcgggagctggagcttcgccagggtcagagctgcggcagcaccctggtagaccgcattcctgatgacccgcggggtgcgctccatgaagaagtgcattcgcccaaccaagtcgagtgggtcgcctggagggggcggggaagcaaaacgttgcatgcacctagcgccctggcagcgagctcctgtagtatcacctgcgtcgcctccagctcatgctcgcaagcctccagggcggcccggcagtgctccaacactttcgcctcctcctacagctccttccacatgcagtcgtgctccgcacgcaccttctccacctttttactcttttctttctcttttcttggcccatctttggtattttcacaaatgtccccctacaaatgataaatcaccaaaactcatggagcttgctagttataaactctaattctaagtttggtgtttatttgagtggattttgtgtgaaagttggtggttagaaataggagttaaggaccgccaacaagatcccccacacttagccctttgctcatcctcgagtaaagttcaaggactaaggtggaacatctcctcaaatggtacgatgcctgcatataagttattccaagcctcacctatacatgtgaactttgaagtgtctaccacgccatcttgggtggttgagaaatggaacagatcagaatccagtcatctttacctctcttgcttagataacttgggtttttgtaaggttttcaaatttaaaacatagtcttgctcctcaaatgattctctcatatagctcaatgtgtatggtttctcaccaaggcaatgttttgcctcttttcatcctacttctaatatttcttttgtggagcttagggtagggaatgaaaaggaagcatacttgcattgcatatgttactaagtcaaaaaccaaatctgaggagaagcaagtcatacaatctgatcaagatgtgcaagtgtgtggatatgtggattaagataactcctgtttattcatgctctcctccttaataaactttagagggcatggcaatctttgcatgggccttcatgagctcatcgtatgtctaagcatggagctcatcatttatataagcatggtgataccaaaattactccttttgagcatgtttatatttaggaggacgttttacctgttgaggtaaatctgaacgctaataaatcggctaagcaaaataatttatcacctgttgattctaacaatttgatgatggacaatattgatgaggtgactgacaaatgattgaaggctttaaaggagattgagaaggataaatctacaataaaaatgtaaagaagaaagcattcaaagtgtgagatctggtgtggaagactattttgcctcttgggggtaaaagacaacaagtttagtaagtggcctcaaaattgggagggcccatgcaagattgttaaagtaattgttttggattgacggaggcatttcaaggtgatcatctacctagagctctcaatgggaggtgctcgaagacatattacccatgtgtatggcaagatgtttagctagtaactgactgatagtgtaaacgatctccaatggggcaagacatattacctaaggccaggctggtttttgcaagttcgagtaggatatagagattctcgtgcgagttgtaaacgatctccaatggggcaagacatcctaccctatatatagtgaaggggcagtagctgattgagaatcaatcaatcaagcacaatataatttattaattttttattcaaacccaatttttttccttttccaaccctaattatagttttccttttgcctctaggacaaattgacgtgttccgggtatcctgctgaattaagaacaaccctaggtgcacctgtcccgatagagtcccacctgggtaggcattcatagggattcgtgtatttcctgcaaaaaagcgattaagctggcttctaaaactggctaggccggattctgtggccttcactaccaggtgattttcatgtgatccgtgcattctagcactttgctatgtaacccaaacttaagtcgacaactataaatatgctacttgcaggatgttatcacgacacaactcctaatctacggaagcctaagtttagttttgctcggagacaagcaattgtggccagtcactatagctacgtcagagggtagtgggagcagttgcgtcgttggattgaaaacaggtggatcgtatcagatattatgcattcacatggacagtaaatgtggtacagtaacttcgcaaacaataaaatctgtcacaatttattagtgcactcctctgacgtaaatgcttctacgtcagaggatttgattccgagggccgctgcacccatcactaatgacggtctttacccatcatcatggaccattgttcacatccatgctatcactgtcgtcctgtccatgcactgcagccctctataaatactggcatccctcccccgttcacagatcacacaacacaagcaagaaataaacggtagctgccataactagtacacc

SEQ ID NO: 25 - DNA, 463 bp, Zea mays
aaggtgcaaggattgctggagcgtcaaggatcattggtgtcgacctgaaccccagcagattcgaagaaggtacagtacacacacatgtatatatgtatgatgtatcccttcgatcgaaggcatgccttggtataatcactgagtagtcattttattactttgttttgacaagtcagtagttcatccatttgtcccattttttcagcttggaagtttggttgcactggccttggtctaataactgagtagtcattttattacgttgtttcgacaagtcagtagctcatccatctgtcccattttttcagctaggaagtttggttgcactggccttggactaataactgattagtcattttattacattgtttcgacaagtcagtagctcatccatctgtcccatttttcagctaggaagttcggttgcactgaatttgtgaacccaaaagaccacaacaagccg

SEQ ID NO: 26 - protein, 21 aa, Hordeum vulgare
SSSSFADSNPIRPVTDRAAST

SEQ ID NO: 27 - protein, 7 aa, Nicotiana tabacum
GLLVDTM

SEQ ID NO: 28 - DNA, 891 bp, Oryza sativa
ggtcattcatatgcttgagaagagagtcgggatagtccaaaataaaacaaaggtaagattacctggtcaaaagtgaaaacatcagttaaaaggtggtataaagtaaaatatcggtaataaaaggtggcccaaagtgaaatttactcttttctactattataaaaattgaggatgtttttgtcggtactttgatacgtcatttttgtatgaattggtttttaagtttattcgcttttggaaatgcatatctgtatttgagtcgggttttaagttcgtttgcttttgtaaatacagagggatttgtataagaaatatctttaaaaaaacccatatgctaatttgacataatttttgagaaaaatatatattcaggcgaattctcacaatgaacaataataagattaaaatagctttcccccgttgcagcgcatgggtattttttctagtaaaaataaaagataaacttagactcaaaacatttacaaaaacaacccctaaagttcctaaagcccaaagtgctatccacgatccatagcaagcccagcccaacccaacccaacccaacccaccccagtccagccaactggacaatagtctccacacccccccactatcaccgtgagttgtccgcacgcaccgcacgtctcgcagccaaaaaaaaaaaaagaaagaaaaaaaagaaaaagaaaaaacagcaggtgggtccgggtcgtgggggccggaaacgcgaggaggatcgcgagccagcgacgaggccggccctccctccgcttccaaagaaacgccccccatcgccactatatacatacccccccctctcctcccatccccccaaccctaccaccaccaccaccaccacctccacctcctcccccctcgctgccggacgac

(19) United States
(12) Patent Application Publication
(10) Pub. No.: US 2014/0068817 A1
(43) Pub. Date: Mar. 6, 2014

(54) EXPRESSION OF ISOMERS OF SUCROSE INCREASES SEED WEIGHT, SEED NUMBER AND/OR SEED SIZE

(71) Applicants: Stefan Dittmar, Noblesville, IN (US); George Singletary, Ankeny, IA (US); Asha Palta, Carmel, IN (US); Barry Stoddard, Bellevue, WA (US); Paul Olivier, Seattle, WA (US)

(72) Inventors: Stefan Dittmar, Noblesville, IN (US); George Singletary, Ankeny, IA (US); Asha Palta, Carmel, IN (US); Barry Stoddard, Bellevue, WA (US); Paul Olivier, Seattle, WA (US)

(73) Assignee: Targeted Growth, Inc., Seattle, WA (US)

(21) Appl. No.: 14/002,823

(22) PCT Filed: Mar. 5, 2012
(86) PCT No.: PCT/US12/27750

§ 371 Date: Nov. 14, 2013

Related U.S. Application Data
(60) Provisional application No. 61/448,980, filed on Mar. 3, 2011.

(51) Int. Cl. C12N 15/82

(52) U.S. Cl. CPC C12N 15/8261
USPC 800/290; 435/320.1; 800/298; 800/312; 800/306; 800/314; 800/317.4; 800/322; 800/320.1; 800/320.2; 800/320.3; 800/320; 435/419; 800/313

(57) ABSTRACT

The present invention provides expression vectors comprising polynucleotides encoding chimeric sucrose isomerases, and methods of using the same. In addition, transgenic plants expressing said chimeric sucrose isomerases are provided. Furthermore, methods of increasing average seed weight, seed number and/or seed size of a plant by using said chimeric sucrose isomerases are provided.

### CROSS-REFERENCE TO RELATED APPLICATIONS

This application claims the benefit of U.S. Provisional Patent Application Ser. No. 61/448,980, filed Mar. 3, 2011, which is hereby incorporated by reference in their entireties for all purposes.

### TECHNICAL FIELD

The invention generally relates to methods for increasing crop yield. More specifically, the present invention relates to methods and compositions for increasing plant seed weight, seed number and/or seed size by expressing one or more sucrose isomerases in the plant.

### BACKGROUND

The most important trait as a target for crop improvement is yield. Efforts to improve crop yields by developing new plant varieties can be divided into two approaches. One is to reduce crop yield losses by breeding or engineering crop varieties with increased resistance to abiotic stress conditions such as drought, cold, or salt or to biotic stress conditions resulting from pests or disease-causing pathogens. While this approach has value, it does not provide fundamentally improved crop yield in the absence of stress conditions and in fact, such resistance may direct plant resources that otherwise would be available for increased yield in the plant. The second approach is to breed or engineer new crop varieties in which the basic yield capacity is increased.

Classical breeding programs have initially produced substantial gains in improved yield in a variety of crops. A commonly experienced pattern though has been substantial gains in yield initially followed by incremental further improvements that become smaller and more difficult to obtain. More recently developed approaches based on molecular biology technologies have in principle offered the potential to achieve substantial improvement in crop yield by altering the timing, location, or level of expression of plant genes or heterologous genes that play a role in plant growth and/or development. Substantial progress has been made over the past twenty years in identifying plant genes and or heterologous genes that have a role in plant growth and/or development. Because of the complexity of plant growth regulation and how it relates in the end to yield traits, it is still not obvious which, if any, of these genes would be a clear candidate to improve crop yield.

Sucrose isomerases have been used previously to engineer the carbohydrate production in plants. For example, sucrose isomerases were previously transformed into tobacco, potato tubers, and sugarcane. Wu et al. (Plant Biotechnol J. 2007 January; 5(1):109-17) reported that expression of sucrose isomerase in sugarcane increases total stored sugar. But such expression is not always beneficial to the plants. For example, expression of a cytosolic version of a sugar isomerase in sugarcane was very detrimental to plant development (Wu et al., Plant Biotechnol J. 2007 January; 5(1):109-17). Additionally, expression of sucrose isomerases in tobacco leads to strong phenotypic alterations, including leaves with curled margins and bleached areas, and dysfunctional flower buds which result in sterility and absence of seed formation (Börnke et al., Planta. 2002 January; 214(3):356-64). Apoplastic targeting of sucrose isomerase in potato tubers produces plants with no change in tuber yield, compared to controls, but leads to decreased sugar content in potato tubers and triggers starch breakdown and respiration in stored potato tubers (Hajirezaei et al., J Exp Bot. 2003 January; 54(382):477-88).

The inventors of the present invention discovered that expressing sucrose isomerases in plants increase the seed yield, more specifically, by increasing the seed weight, seed number and/or seed size. This result is totally unexpected based on previous studies of expressing sucrose isomerases in plants.

### SUMMARY

The present invention provides expression vectors comprising a polynucleotide having a nucleic acid sequence encoding a sucrose isomerase, or biologically active variants, or fragments thereof. In some embodiments, the polynucleotide is operably-linked to a stem specific and/or stem preferred promoter. In some embodiments, the sucrose isomerase is derived from microbes or insects, such as, for example, the sucrose isomerases identified in Agrobacterium radiobacter, Pantoea dispersa, Protaminobacter rubrum, Erwinia rhapontici, Serratia plyinuthica, Pseudomonas mesoacidophila, Klebsiella planticola, Enterobacter sp., and Bemisia argentifolii. In some embodiments, the sucrose isomerase is any biologically active chimeric sucrose isomerase designed in silico using gene shuffling and/or directed molecular evolution.

In some embodiments, the sucrose isomerase is encoded by a polynucleotide sequence that can be selected from the group consisting of: (i) SEQ ID NOs: 1, 3, 7, 9, 14, biologically active variants, and fragments thereof; (ii) a sequence sharing at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% identity to a gene encoding the polypeptide of SEQ ID NO: 2, 4, 8, 10, 13 or 15, wherein the sequence encodes a functional sucrose isomerase, or biologically active variants, or fragments thereof; and (iii) a sequence that can hybridize under stringent conditions with a nucleic acid sequence encoding the polypeptide of SEQ ID NO 2, 4, 8, 10, 13 or 15, or biologically active variants, or fragments thereof. In some embodiments, the nucleic acid sequence encoding sucrose isomerase has been codon-optimized for plant expression.

In some embodiments, the stem specific and/or stem preferred promoter is a stalk specific promoter. In one example, the stem specific and/or stem preferred promoter can be selected from the group consisting of promoters of the cellulose synthesis catalytic complex involved in the synthesis of cellulose during plant secondary cell wall formation in the plant vascular tissue (stem tissue), storage protein promoters, promoters associated with early nodulin genes (e.g., rice ENOD40 promoter, homologs, orthologs, functional variants or fragments thereof), o-methyl transferase (OMT) promoter (e.g., sugarcane o-methyl transferase promoter, homologs, orthologs, functional variants or fragments thereof), jasmonate-inducible protein promoter (e.g., sugarcane jasmonate-inducible protein promoter, homologs, orthologs, functional variants or fragments thereof), SCSV3, SCSV4, SCSV5, and SCSV7 promoters, the rolC gene promoter of Agrobacterium rhizogenes, the rolA gene promoter of Agrobacterium rhizogenes, the promoter of the Agrobacterium tumefaciens T-DNA gene 5, the rice sucrose synthase RSs1 gene promoter, the CoYMV or Commelina yellow mottle badnavirus promoter, the CFDV or coconut foliar decay virus promoter, the RTBV or rice tungro bacilliform virus promoter, the pea glutamine synthase GS3A gene promoter, the invCD111 and invCD141 promoters of the potato invertase genes, the VAHOX1 promoter, the pea cell wall invertase gene promoter, the promoter of chitinase, acid invertase gene promoter from carrot, the promoter of the sulfate transporter gene Sultr1;3, a promoter of a sucrose synthase gene, promoter of a tobacco sucrose transporter gene, functional variants thereof, fragments thereof, and any promoter homologous to (or having a high sequence identity to) any of these promoters.

In some embodiments, the polynucleotide having a nucleic acid sequence encoding the sucrose isomerase is in-frame tagged with a subcellular localization signal peptide. In some embodiments, the subcellular localization signal peptide is an N-terminal or C-terminal signal peptide. In further embodiments, the signal peptide is an ER signal peptide, a vacuolar targeting signal peptide, or combination thereof. In some embodiments, the vacuolar targeting signal peptide can be selected from the group consisting of N′ terminal propeptide (NTPP) of sweet potato sporamin, 21 amino acids of barley proaleurain, 7 amino acids of tobacco chitinase A, signal peptide of barley lectin, signal peptide of tobacco glucanase, signal peptide of tobacco osmotin, and signal peptide of 2S albumin storage proteins from Brazil nut. In some embodiments, the ER signal peptide can be selected from the group consisting of ER signal peptide from sweet potato sporamin, ER signal peptide from fava bean lectin, barley hordein, tomato polygalacturonase, wheat high molecular weight glutenin, soybean protein VSPαS, chitinase, oleosin, patatin, 2s albumin of Bertbolletia excelsa H.B.K., proteinase inhibitor II, ER retention signal (KDEL or HDEL), functional variants thereof, and fragments thereof.

In further embodiments, the expression vectors of the present invention can further comprise a plant gene intron sequence, wherein the plant gene intron sequence is between the plant promoter and the polynucleotide encoding the sucrose isomerase, and wherein the intron sequence leads to intron-mediated enhancement (IME) of sucrose isomerase expression. In some embodiments, the plant gene intron is the first intron of maize ADH1 gene, or functional variants thereof, or fragment thereof.

In another aspect, the present invention provides methods for increasing seed weight, seed number and/or seed size in a plant comprising incorporating into the plant one or more transgenes comprising a polynucleotide having a nucleic acid sequence encoding a sucrose isomerase, or biologically active variants, or fragments thereof. In some embodiments, the transgene is operably-linked to a stem specific and/or stem preferred promoter. In some embodiments, the stem specific or the stem preferred promoter is a promoter associated with early nodulin genes (e.g., rice ENOD40 promoter, homologs, orthologs, functional variants or fragments thereof), or an o-methyl transferase (OMT) promoter (e.g., sugarcane o-methyl transferase promoter, homologs, orthologs, functional variants or fragments thereof), or a jasmonate-inducible protein promoter (e.g., sugarcane jasmonate-inducible protein promoter). In some embodiments, the plant is a dicotyledon plant or a monocotyledon plant.

In some embodiments, the expression of a sucrose isomerase increases seed weight by primarily increasing seed test weight. In some embodiments, the expression of a sucrose isomerase increases seed number by primarily increase average seed number per plant or per acre.

In some embodiments, the sucrose isomerase is encoded by a polynucleotide sequence selected from the group consisting of:

(i) SEQ ID NOs: 1, 3, 7, 9, 14, biologically active variants, and fragments thereof;
(ii) a sequence sharing at least 65% identity to a gene encoding the polypeptide of SEQ ID NO: 2, 4, 8, 10, 13, or 15 wherein the sequence encodes a functional sucrose isomerase, biologically active variants, and fragments thereof; and
(iii) a sequence that can hybridize under stringent condition with a nucleic acid sequence encoding the polypeptide of SEQ ID NO 2, 4, 8, 10, 13 or 15, or biologically active variants, or fragments thereof; and optionally, wherein the polynucleotide sequence is codon-optimized for plant expression.

In some embodiments, the plant can be a dicotyledon plant selected from the group consisting of bean, soybean, peanut, nuts, Arabidopsis, Brassicaceae family (Camelina, oilseed rape, Canola, etc.), amaranth, cotton, peas, tomatoes, sugarbeet, and sunflower. In other embodiments, the plant can be a monocotyledon plant selected from the group consisting of corn, rice, wheat, barley, sorghum, millets, oats, ryes, triticales, buckwheats, fonio, quinoa, or oil palm.

In some embodiments, the compositions and methods of the present invention can increase the average seed weight, seed number and/or seed size of a plant by at least 5%, at least 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 110%, 120%, 130%, 140%, 150%, 160%, 170%, 180%, 190%, 200%, 210%, 220%, 230%, 240%, 250%, 260%, 270%, 280%, 290%, 300%, 310%, 320%, 330%, 340%, 350%, 360%. 370%, 380%, 390%, 400%, or greater when compared to a control plant not expressing the sucrose isomerase, or biologically active variants, or fragments thereof. The control plant can be a wild-type plant with or without any transgene inside.

In another aspect, the present invention provides a transgenic plant expressing a sucrose isomerase, biologically active variants, or fragments thereof, wherein the transgenic plant has increased seed weight, seed number and/or seed size compared to a control plant not expressing the sucrose isomerase, biologically active variants, or fragments thereof. In some embodiments, said transgenic plant is produced by incorporating a recombinant transgenic sucrose isomerase construct of the present invention into a plant. In some embodiments, the recombinant sucrose isomerase is under the control of a stem specific and/or stem preferred promoter, e.g., a stalk specific promoter. In some embodiments, the transgenic plant is a dicotyledon plant or a monocotyledon plant.

In some embodiments, the transgenic plants expressing recombinant sucrose isomerase is produced by transforming the expression vectors of the present invention into a plant. In another embodiment, the transgenic plants expressing recombinant sucrose isomerase are produced from progenies genetically related to transgenic plants transformed with the expression vectors of the present invention.

In some embodiments, the transgenic plants of the present invention have an increased seed weight, seed number and/or seed size at least 5%, at least 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 110%, 120%, 130%, 140%, 150%, 160%, 170%, 180%, 190%, 200%, 210%, 220%, 230%, 240%, 250%, 260%, 270%, 280%, 290%, 300%, 310%, 320%, 330%, 340%, 350%, 360%, 370%, 380%, 390%, 400%, or more when compared to a control plant not expressing sucrose isomerase, biologically active variants, or fragments thereof. The control plant can be a wild-type plant with or without any transgene inside.

The present invention also provides a seed, a fruit, a plant part, or a plant cell of the transgenic plants of the present invention. In some embodiments, said plant cell is an embryo, apollen or an ovule of the transgenic plants. The present invention also provides a genetically related plant population comprising the transgenic plants of the present invention. In addition, the present invention also provides tissue culture of regenerable cells of the transgenic plants. In some embodiments, the tissue culture is made from regenerable cells that are derived from embryos, protoplasts, meristematic cells, callus, pollen, leaves, anthers, stems, petioles, roots, root tips, fruits, seeds, flowers, cotyledons, and/or hypocotyls.

### BRIEF DESCRIPTION OF THE DRAWINGS

FIG. 1 depicts recombinant vector TG Zm78+pSB1 for expressing sucrose isomerase MutB gene in maize. The MutB gene is driven by the OMT promoter. The ADH1 intron was introduced right before the translation initiation site. The nucleic acid sequences encoding the ER and the NTPP signal peptides were connected to the N-terminus of the codon-optimized MutB gene.

FIG. 2 depicts recombinant vector TG Zm75+pSB1 for expressing sucrose isomerase UQ68J gene in maize. The UQ68J gene is driven by the OsENOD40 promoter. The ADH1 intron was introduced right before the translation initiation site. The nucleic acid sequences encoding the ER and the NTPP signal peptides were connected to the N-terminus of the codon-optimized UQ68J gene.

FIG. 3 depicts recombinant vector TG Zm77+pSB1 for expressing modified sucrose isomerase UQ68J gene in maize. The modified UQ68J gene is driven by the OsENOD40 promoter. The ADH1 intron was introduced right before the translation initiation site. The nucleic acid sequences encoding the ER and the NTPP signal peptides were connected to the N-terminus of the codon-optimized modified UQ68J gene.

FIG. 4 depicts recombinant vector TG Zm81+pSB1 for expressing sucrose isomerase MutB gene in maize. The MutB gene is driven by the OsENOD40 promoter. The ADH1 intron was introduced right before the translation initiation site. The nucleic acid sequences encoding the ER and the NTPP signal peptides were connected to the N-terminus of the codon-optimized MutB gene.

FIG. 5 depicts recombinant vector TG Zm88+pSB1 for expressing sucrose isomerase UQ68J gene in maize. The UQ68J gene is driven by the JAS promoter. The ADH1 intron was introduced right before the translation initiation site. The nucleic acid sequences encoding the ER and the NTPP signal peptides were connected to the N-terminus of the codon-optimized UQ68J gene.

FIG. 6 depicts average seed weight (mg/kernel, air-dry basis) of maize plants expressing sucrose isomerase genes (TG75, TG77, TG78, TG81, TG88) and maize plants expressing non-sucrose isomerase genes (TG74, TG84, TG85, TG87).

FIG. 7 depicts recombinant vector TG Zm54+pSB1 for expressing sucrose isomerase UQ68J gene in maize. The UQ68J gene is driven by the rice Actin promoter (SEQ ID NO. 28). The ADH1 intron was introduced right before the translation initiation site. The nucleic acid sequences encoding the ER and the NTPP signal peptides were connected to the N-terminus of the codon-optimized UQ68J gene.

FIG. 8 depicts recombinant vector TG Zm55+pSB1 for expressing modified sucrose isomerase UQ68J gene in maize. The modified UQ68J gene is driven by the rice Actin promoter (SEQ ID NO. 28). The ADH1 intron was introduced right before the translation initiation site. The nucleic acid sequences encoding the ER and the NTPP signal peptides were connected to the N-terminus of the codon-optimized modified UQ68J gene.

FIG. 9 depicts recombinant vector TG Zm60+pSB1 for expressing sucrose isomerase MutB gene in maize. The MutB gene is driven by the rice Actin promoter (SEQ ID NO. 28). The ADH1 intron was introduced right before the translation initiation site. The nucleic acid sequences encoding the ER and the NTPP signal peptides were connected to the N-terminus of the codon-optimized MutB gene.

FIG. 10 depicts recombinant vector TG Zm58+pSB1 for expressing modified sucrose isomerase UQ68J gene in maize. The modified UQ68J gene is driven by the OMT promoter. The ADH1 intron was introduced right before the translation initiation site. The nucleic acid sequences encoding the ER and the NTPP signal peptides were connected to the N-terminus of the codon-optimized modified UQ68J gene.

FIG. 11 depicts recombinant vector TG Zm155+pSB1 for expressing sucrose isomerase MutB gene in maize. The MutB gene is driven by the OsENOD40 promoter. The ADH1 intron was introduced right before the translation initiation site. The nucleic acid sequences encoding the ER and the NTPP signal peptides were connected to the N-terminus of the codon-optimized MutB gene.

FIG. 12 depicts the percent of null ear grain weight for TG_Zm 155 Events across all locations. Black data points are not statistically significant. Gray-shaded points above and below the dashed line at “100% of Null Ear Grain Wt.” signify statistically significant (p<0.10) increases or decreases, respectively, for the grain yield trait.

FIG. 13 depicts the percent of null ear grain weight for TG_Zm 54, TG_Zm 58, TG_Zm 75, TG_Zm 77, TG_Zm 78 and TG_Zm 81 Events across all locations. Black data points are not statistically significant. Gray-shaded points above and below the dashed line at “100% of Null Ear Grain Wt.” signify statistically significant (p<0.10) increases or decreases, respectively, for the grain yield trait.

### SEQUENCES

Sequence listings for SEQ ID No: 1-SEQ ID No: 28 are part of this application and are incorporated by reference herein. Sequence listings are provided at least at the end of this document.

### DETAILED DESCRIPTION

All publications, patents and patent applications, including any drawings and appendices, and all nucleic acid sequences and polypeptide sequences identified by GenBank Accession numbers, herein are incorporated by reference to the same extent as if each individual publication or patent application was specifically and individually indicated to be incorporated by reference.

The following description includes information that may be useful in understanding the present invention. It is not an admission that any of the information provided herein is prior art or relevant to the presently claimed inventions, or that any publication specifically or implicitly referenced is prior art.

### DEFINITIONS

As used herein, the verb “comprise” as is used in this description and in the claims and its conjugations are used in its non-limiting sense to mean that items following the word are included, but items not specifically mentioned are not excluded.

As used herein, the term “plant” refers to any living organism belonging to the kingdom Plantae (i.e., any genus/species in the Plant Kingdom). This includes familiar organisms such as but not limited to trees, herbs, bushes, grasses, vines, ferns, mosses and green algae. The term refers to both monocotyledonous plants, also called monocots, and dicotyledonous plants, also called dicots. Examples of particular plants include but are not limited to corn, potatoes, roses, apple trees, sunflowers, wheat, rice, bananas, tomatoes, opo, pumpkins, squash, lettuce, cabbage, oak trees, guzmania, geraniums, hibiscus, clematis, poinsettias, sugarcane, taro, duck weed, pine trees, Kentucky blue grass, zoysia, coconut trees, brassica leafy vegetables (e.g. broccoli, broccoli raab, Brussels sprouts, cabbage, Chinese cabbage (Bok Choy and Napa), cauliflower, cavalo, collards, kale, kohlrabi, mustard greens, rape greens, and other brassica leafy vegetable crops), bulb vegetables (e.g. garlic, leek, onion (dry bulb, green, and Welch), shallot, and other bulb vegetable crops), citrus fruits (e.g. grapefruit, lemon, lime, orange, tangerine, citrus hybrids, pummelo, and other citrus fruit crops), cucurbit vegetables (e.g. cucumber, citron melon, edible gourds, gherkin, muskmelons (including hybrids and/or cultivars of cucumis melons), water-melon, cantaloupe, and other cucurbit vegetable crops), fruiting vegetables (including eggplant, ground cherry, pepino, pepper, tomato, tomatillo, and other fruiting vegetable crops), grape, leafy vegetables (e.g. romaine), root/tuber and corm vegetables (e.g. potato), and tree nuts (almond, pecan, pistachio, and walnut), berries (e.g., tomatoes, barberries, currants, elderberries, gooseberries, honeysuckles, mayapples, nannyberries, Oregon-grapes, see-buckthorns, hackberries, bearberries, lingonberries, strawberries, sea grapes, lackberries, cloudberries, loganberries, raspberries, salmonberries, thimbleberries, and wineberries), cereal crops (e.g., corn, rice, wheat, barley, sorghum, millets, oats, ryes, triticales, buckwheats, fonio, quinoa, oil palm), pome fruit (e.g., apples, pears), stone fruits (e.g., coffees, jujubes, mangos, olives, coconuts, oil palms, pistachios, almonds, apricots, cherries, damsons, nectarines, peaches and plums), vine (e.g., table grapes, wine grapes), fiber crops (e.g. hemp, cotton), ornamentals, and the like.

As used herein, the term “plant part” refers to any part of a plant including but not limited to the shoot, root, stem, seeds, stipules, leaves, petals, flowers, ovules, bracts, branches, petioles, internodes, bark, pubescence, tillers, rhizomes, fronds, blades, pollen, stamen, and the like. The two main parts of plants grown in some sort of media, such as soil, are often referred to as the “above-ground” part, also often referred to as the “shoots”, and the “below-ground” part, also often referred to as the “roots”.

The term “a” or “an” refers to one or more of that entity; for example, “a gene” refers to one or more genes or at least one gene. As such, the terms “a” (or “an”), “one or more” and “at least one” are used interchangeably herein. In addition, reference to “an element” by the indefinite article “a” or “an” does not exclude the possibility that more than one of the elements are present, unless the context clearly requires that there is one and only one of the elements.

As used herein, the term “chimeric protein” or “recombinant protein” refers to a construct that links at least two heterologous proteins into a single macromolecule (fusion protein).

As used herein, the term “nucleic acid” refers to a polymeric form of nucleotides of any length, either ribonucleotides or deoxyribonucleotides, or analogs thereof. This term refers to the primary structure of the molecule, and thus includes double- and single-stranded DNA, as well as double- and single-stranded RNA. It also includes modified nucleic acids such as methylated and/or capped nucleic acids, nucleic acids containing modified bases, backbone modifications, and the like. The terms “nucleic acid” and “nucleotide sequence” are used interchangeably.

As used herein, the terms “polypeptide,” “peptide,” and “protein” are used interchangeably herein to refer to polymers of amino acids of any length. These terms also include proteins that are post-translationally modified through reactions that include glycosylation, acetylation and phosphorylation.

As used herein, the term “homologous” or “homologue” or “ortholog” is known in the art and refers to related sequences that share a common ancestor or family member and are determined based on the degree of sequence identity. The terms “homology”, “homologous”, “substantially similar” and “corresponding substantially” are used interchangeably herein. They refer to nucleic acid fragments wherein changes in one or more nucleotide bases do not affect the ability of the nucleic acid fragment to mediate gene expression or produce a certain phenotype. These terms also refer to modifications of the nucleic acid fragments of the instant invention such as deletion or insertion of one or more nucleotides that do not substantially alter the functional properties of the resulting nucleic acid fragment relative to the initial, unmodified fragment. It is therefore understood, as those skilled in the art will appreciate, that the invention encompasses more than the specific exemplary sequences. These terms describe the relationship between a gene found in one species, subspecies, variety, cultivar or strain and the corresponding or equivalent gene in another species, subspecies, variety, cultivar or strain. For purposes of this invention homologous sequences are compared. “Homologous sequences” or “homologues” or “orthologs” are thought, believed, or known to be functionally related. A functional relationship may be indicated in any one of a number of ways, including, but not limited to: (a) degree of sequence identity and/or (b) the same or similar biological function. Preferably, both (a) and (b) are indicated. The degree of sequence identity may vary, but in some embodiments, is at least 50% (when using standard sequence alignment programs known in the art), at least 60%, at least 65%, at least 70%, at least 75%, at least 80%, at least 85%, at least 90%, at least about 91%, at least about 92%, at least about 93%, at least about 94%, at least about 95%, at least about 96%, at least about 97%, at least about 98%, or at least 98.5%, or at least about 99%, or at least 99.5%, or at least 99.8%, or at least 99.9%. Homology can be determined using software programs readily available in the art, such as those discussed in Current Protocols in Molecular Biology (F. M. Ausubel et al., eds., 1987) Supplement 30, section 7.718, Table 7.71. Some alignment programs are MacVector (Oxford Molecular Ltd, Oxford, U.K.), ALIGN Plus (Scientific and Educational Software, Pennsylvania) and AlignX (Vector NTI, Invitrogen, Carlsbad, Calif.). Another alignment program is Sequencher (Gene Codes, Ann Arbor, Mich.), using default parameters.

As used herein, the term “nucleotide change” refers to, e.g., nucleotide substitution, deletion, and/or insertion, as is well understood in the art. For example, mutations contain alterations that produce silent substitutions, additions, or deletions, but do not alter the properties or activities of the encoded protein or how the proteins are made.

As used herein, the term “protein modification” refers to, e.g., amino acid substitution, amino acid modification, deletion, and/or insertion, as is well understood in the art,

As used herein, the term “derived from” refers to the origin or source, and may include naturally occurring, recombinant, unpurified, or purified molecules. A nucleic acid or an amino acid derived from an origin or source may have all kinds of nucleotide changes or protein modification as defined elsewhere herein.

As used herein, the term “agent”, as used herein, means a biological or chemical compound such as a simple or complex organic or inorganic molecule, a peptide, a protein or an oligonucleotide that modulates the function of a nucleic acid or polypeptide. A vast array of compounds can be synthesized, for example oligomers, such as oligopeptides and oligonucleotides, and synthetic organic and inorganic compounds based on various core structures, and these are also included in the term “agent”. In addition, various natural sources can provide compounds for screening, such as plant or animal extracts, and the like. Compounds can be tested singly or in combination with one another.

As used herein, the term “at least a portion” or “fragment” of a nucleic acid or polypeptide means a portion having the minimal size characteristics of such sequences, or any larger fragment of the full length molecule, up to and including the full length molecule. For example, a portion of a nucleic acid may be 12 nucleotides, 13 nucleotides, 14 nucleotides, 15 nucleotides, 16 nucleotides, 17 nucleotides, 18 nucleotides, 19 nucleotides, 20 nucleotides, 22 nucleotides, 24 nucleotides, 26 nucleotides, 28 nucleotides, 30 nucleotides, 32 nucleotides, 34 nucleotides, 36 nucleotides, 38 nucleotides, 40 nucleotides, 45 nucleotides, 50 nucleotides, 55 nucleotides, and so on, going up to the full length nucleic acid. Similarly, a portion of a polypeptide may be 4 amino acids, 5 amino acids, 6 amino acids, 7 amino acids, and so on, going up to the full length polypeptide. The length of the portion to be used will depend on the particular application. A portion of a nucleic acid useful as hybridization probe may be as short as 12 nucleotides; in some embodiments, it is 20 nucleotides. A portion of a polypeptide useful as an epitope may be as short as 4 amino acids. A portion of a polypeptide that performs the function of the full-length polypeptide would generally be longer than 4 amino acids.

As used herein, “sequence identity” or “identity” in the context of two nucleic acid or polypeptide sequences includes reference to the residues in the two sequences which are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. Where sequences differ in conservative substitutions, the percent sequence identity may be adjusted upwards to correct for the conservative nature of the substitution. Sequences which differ by such conservative substitutions are said to have “sequence similarity” or “similarity.” Means for making this adjustment are well-known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., according to the algorithm of Meyers and Miller, Computer Applic. Biol. Sci., 4:11-17 (1988).

As used herein, the term “substantially complementary” means that two nucleic acid sequences have at least about 65%, preferably about 70% or 75%, more preferably about 80% or 85%, even more preferably 90% or 95%, and most preferably about 98% or 99%, sequence complementarities to each other. This means that primers and probes must exhibit sufficient complementarity to their template and target nucleic acid, respectively, to hybridize under stringent conditions. Therefore, the primer and probe sequences need not reflect the exact complementary sequence of the binding region on the template and degenerate primers can be used. For example, a non-complementary nucleotide fragment may be attached to the 5′-end of the primer, with the remainder of the primer sequence being complementary to the strand. Alternatively, non-complementary bases or longer sequences can be interspersed into the primer, provided that the primer has sufficient complementarity with the sequence of one of the strands to be amplified to hybridize therewith, and to thereby form a duplex structure which can be extended by polymerizing means. The non-complementary nucleotide sequences of the primers may include restriction enzyme sites. Appending a restriction enzyme site to the end(s) of the target sequence would be particularly helpful for cloning of the target sequence. A substantially complementary primer sequence is one that has sufficient sequence complementarity to the amplification template to result in primer binding and second-strand synthesis. The skilled person is familiar with the requirements of primers to have sufficient sequence complementarity to the amplification template.

As used herein, the terms “polynucleotide”, “polynucleotide sequence”, “nucleic acid sequence”, “nucleic acid fragment”, and “isolated nucleic acid fragment” are used interchangeably herein. These terms encompass nucleotide sequences and the like. A polynucleotide may be a polymer of RNA or DNA that is single- or double-stranded, that optionally contains synthetic, non-natural or altered nucleotide bases. A polynucleotide in the form of a polymer of DNA may be comprised of one or more segments of cDNA, genomic DNA, synthetic DNA, or mixtures thereof. Nucleotides (usually found in their 5′-monophosphate form) are referred to by a single letter designation as follows: “A” for adenylate or deoxyadenylate (for RNA or DNA, respectively), “C” for cytidylate or deoxycytidylate, “G” for guanylate or deoxyguanylate, “U” for uridylate, “T” for deoxythymidylate, “R” for purines (A or G), “Y” for pyrimidines (C or T), “K” for G or T, “H” for A or C or T, “I” for inosine, and “N” for any nucleotide.

As used herein, the phrase “a biologically active variant” or “functional variant” with respect to a protein refers to an amino acid sequence that is altered by one or more amino acids with respect to a reference sequence, while still maintains substantial biological activity of the reference sequence. The variant can have “conservative” changes, wherein a substituted amino acid has similar structural or chemical properties, e.g., replacement of leucine with isoleucine. Alternatively, a variant can have “nonconservative” changes, e.g., replacement of a glycine with a tryptophan. Analogous minor variations can also include amino acid deletion or insertion, or both. Guidance in determining which amino acid residues can be substituted, inserted, or deleted without eliminating biological or immunological activity can be found using computer programs well known in the art, for example, DNASTAR software.

The term “primer” as used herein refers to an oligonucleotide which is capable of annealing to the amplification target allowing a DNA polymerase to attach, thereby serving as a point of initiation of DNA synthesis when placed under conditions in which synthesis of primer extension product is induced, i.e., in the presence of nucleotides and an agent for polymerization such as DNA polymerase and at a suitable temperature and pH. The (amplification) primer is preferably single stranded for maximum efficiency in amplification. Preferably, the primer is an oligodeoxyribonucleotide. The primer must be sufficiently long to prime the synthesis of extension products in the presence of the agent for polymerization. The exact lengths of the primers will depend on many factors, including temperature and composition (A/T vs. G/C content) of primer. A pair of bi-directional primers consists of one forward and one reverse primer as commonly used in the art of DNA amplification such as in PCR amplification.

The terms “stringency” or “stringent hybridization conditions” refer to hybridization conditions that affect the stability of hybrids, e.g., temperature, salt concentration, pH, formamide concentration and the like. These conditions are empirically optimized to maximize specific binding and minimize non-specific binding of primer or probe to its target nucleic acid sequence. The terms as used include reference to conditions under which a probe or primer will hybridize to its target sequence, to a detectably greater degree than other sequences (e.g. at least 2-fold over background). Stringent conditions are sequence dependent and will be different in different circumstances. Longer sequences hybridize specifically at higher temperatures. Generally, stringent conditions are selected to be about 5° C. lower than the thermal melting point (Tm) for the specific sequence at a defined ionic strength and pH. The Tm is the temperature (under defined ionic strength and pH) at which 50% of a complementary target sequence hybridizes to a perfectly matched probe or primer. Typically, stringent conditions will be those in which the salt concentration is less than about 1.0 M Na⁺ ion, typically about 0.01 to 1.0 M Na+ ion concentration (or other salts) at pH 7.0 to 8.3 and the temperature is at least about 30° C. for short probes or primers (e.g. 10 to 50 nucleotides) and at least about 60° C. for long probes or primers (e.g. greater than 50 nucleotides). Stringent conditions may also be achieved with the addition of destabilizing agents such as formamide. Exemplary low stringent conditions or “conditions of reduced stringency” include hybridization with a buffer solution of 30% formamide, 1 M NaCl, 1% SDS at 37° C. and a wash in 2×SSC at 40° C. Exemplary high stringency conditions include hybridization in 50% formamide, 1M NaCl, 1% SDS at 37° C., and a wash in 0.1×SSC at 60° C. Hybridization procedures are well known in the art and are described by e.g. Ausubel et al., 1998 and Sambrook et al., 2001.

As used herein, “coding sequence” refers to a DNA sequence that codes for a specific amino acid sequence. “Regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence.

As used herein, “regulatory sequences” may include, but are not limited to, promoters, translation leader sequences, introns, and polyadenylation recognition sequences.

As used herein, “promoter” refers to a DNA sequence capable of controlling the expression of a coding sequence or functional RNA. The promoter sequence consists of proximal and more distal upstream elements, the latter elements often referred to as enhancers. Accordingly, an “enhancer” is a DNA sequence that can stimulate promoter activity, and may be an innate element of the promoter or a heterologous element inserted to enhance the level or tissue specificity of a promoter. Promoters may be derived in their entirety from a native gene, or be composed of different elements derived from different promoters found in nature, or even comprise synthetic DNA segments. It is understood by those skilled in the art that different promoters may direct the expression of a gene in different tissues or cell types, or at different stages of development, or in response to different environmental conditions. It is further recognized that since in most cases the exact boundaries of regulatory sequences have not been completely defined, DNA fragments of some variation may have identical promoter activity.

As used herein, a “plant promoter” is a promoter capable of initiating transcription in plant cells whether or not its origin is a plant cell, e.g. it is well known that Agrobactenum promoters are functional in plant cells. Thus, plant promoters include promoter DNA obtained from plants, plant viruses and bacteria such as Agrobacterium and Bradyrhizobium bacteria. A plant promoter can be a constitutive promoter or a non-constitutive promoter.

As used herein, a “constitutive promoter” is a promoter which is active under most conditions and/or during most development stages. There are several advantages to using constitutive promoters in expression vectors used in plant biotechnology, such as: high level of production of proteins used to select transgenic cells or plants; high level of expression of reporter proteins or scorable markers, allowing easy detection and quantification; high level of production of a transcription factor that is part of a regulatory transcription system; production of compounds that requires ubiquitous activity in the plant; and production of compounds that are required during all stages of plant development. Non-limiting exemplary constitutive promoters include, CaMV 35S promoter, opine promoters, ubiquitin promoter, actin promoter, alcohol dehydrogenase promoter, etc.

As used herein, a “non-constitutive promoter” is a promoter which is active under certain conditions, in certain types of cells, and/or during certain development stages. For example, tissue specific, tissue preferred, cell type specific, cell type preferred, inducible promoters, and promoters under development control are non-constitutive promoters. Examples of promoters under developmental control include promoters that preferentially initiate transcription in certain tissues, such as stems, leaves, roots, or seeds.

As used herein, “inducible” or “repressible” promoter is a promoter which is under chemical or environmental factors control. Examples of environmental conditions that may effect transcription by inducible promoters include anaerobic conditions, or certain chemicals, or the presence of light.

As used herein, a “tissue specific” promoter is a promoter that initiates transcription only in certain tissues. Unlike constitutive expression of genes, tissue-specific expression is the result of several interacting levels of gene regulation. As such, in the art sometimes it is preferable to use promoters from homologous or closely related plant species to achieve efficient and reliable expression of transgenes in particular tissues. This is one of the main reasons for the large amount of tissue-specific promoters isolated from particular plants and tissues found in both scientific and patent literature. Non-limiting tissue specific promoters include, beta-amylase gene or barley hordein gene promoters (for seed gene expression), tomato pz7 and pz130 gene promoters (for ovary gene expression), tobacco RD2 gene promoter (for root gene expression), banana TRX promoter and melon actin promoter (for fruit gene expression), and embryo specific promoters, e.g., a promoter associated with an amino acid permease gene (AAPI), an oleate 12-hydroxylase:desaturase gene from Lesquerella fendleri (LFAH12), an 2S2 albumin gene (2S2), a fatty acid elongase gene (FAE1), or a leafy cotyledon gene (LEC2).

As used herein, a “tissue preferred” promoter is a promoter that initiates transcription mostly, but not necessarily entirely or solely in certain tissues.

As used herein, a “cell type specific” promoter is a promoter that primarily drives expression in certain cell types in one or more organs, for example, vascular cells in roots, leaves, stalk cells, and stem cells.

As used herein, a “cell type preferred” promoter is a promoter that primarily drives expression mostly, but not necessarily entirely or solely in certain cell types in one or more organs, for example, vascular cells in roots, leaves, stalk cells, and stem cells.

As used herein, a “stem specific” or a “stalk specific” promoter is a promoter that initiates transcription only in stem tissues or stalk tissue.

As used herein, a “stem preferred” or a “stalk preferred” promoter is a promoter that initiates transcription mostly, but not necessarily entirely or solely in stem or stalk tissues.

As used herein, a “vascular specific” promoter is a promoter which initiates transcription only in vascular cells.

As used herein, a “vascular preferred” promoter is a promoter which initiates transcription mostly, but not necessarily entirely or solely in vascular cells.

As used herein, a “phloem specific promoter” is a promoter which initiates transcription only in phloem cells.

As used herein, a “phloem preferred promoter” is a promoter which initiates transcription mostly, but not necessarily entirely or solely in phloem cells.

As used herein, the “3′ non-coding sequences” or “3′ untranslated regions” refer to DNA sequences located downstream of a coding sequence and include polyadenylation recognition sequences and other sequences encoding regulatory signals capable of affecting mRNA processing or gene expression. The polyadenylation signal is usually characterized by affecting the addition of polyadenylic acid tracts to the 3′ end of the mRNA precursor. The use of different 3′ non-coding sequences is exemplified by Ingelbrecht, I. L., et al. (1989) Plant Cell 1:671-680.

As used herein, “RNA transcript” refers to the product resulting from RNA polymerase-catalyzed transcription of a DNA sequence. When the RNA transcript is a perfect complementary copy of the DNA sequence, it is referred to as the primary transcript. An RNA transcript is referred to as the mature RNA when it is an RNA sequence derived from post-transcriptional processing of the primary transcript. “Messenger RNA (mRNA)” refers to the RNA that is without introns and that can be translated into protein by the cell. “cDNA” refers to a DNA that is complementary to and synthesized from an mRNA template using the enzyme reverse transcriptase. The cDNA can be single-stranded or converted into the double-stranded form using the Klenow fragment of DNA polymerase I. “Sense” RNA refers to RNA transcript that includes the mRNA and can be translated into protein within a cell or in vitro. “Antisense RNA” refers to an RNA transcript that is complementary to all or part of a target primary transcript or mRNA, and that blocks the expression of a target gene (U.S. Pat. No. 5,107,065). The complementarity of an antisense RNA may be with any part of the specific gene transcript, i.e., at the 5′ non-coding sequence, 3′ non-coding sequence, introns, or the coding sequence. “Functional RNA” refers to antisense RNA, ribozyme RNA, or other RNA that may not be translated but yet has an effect on cellular processes. The terms “complement” and “reverse complement” are used interchangeably herein with respect to mRNA transcripts, and are meant to define the antisense RNA of the message.

As used herein, the term “operably linked” refers to the association of nucleic acid sequences on a single nucleic acid fragment so that the function of one is regulated by the other. For example, a promoter is operably linked with a coding sequence when it is capable of regulating the expression of that coding sequence (i.e., that the coding sequence is under the transcriptional control of the promoter). Coding sequences can be operably linked to regulatory sequences in a sense or antisense orientation. In another example, the complementary RNA regions of the invention can be operably linked, either directly or indirectly, 5′ to the target mRNA, or 3′ to the target mRNA, or within the target mRNA, or a first complementary region is 5′ and its complement is 3′ to the target mRNA.

As used herein, the term “recombinant” refers to an artificial combination of two otherwise separated segments of sequence, e.g., by chemical synthesis or by the manipulation of isolated segments of nucleic acids by genetic engineering techniques.

As used herein, the phrases “recombinant construct”, “expression construct”, “chimeric construct”, “construct”, and “recombinant DNA construct” are used interchangeably herein. A recombinant construct comprises an artificial combination of nucleic acid fragments, e.g., regulatory and coding sequences that are not found together in nature. For example, a chimeric construct may comprise regulatory sequences and coding sequences that are derived from different sources, or regulatory sequences and coding sequences derived from the same source, but arranged in a manner different than that found in nature. Such construct may be used by itself or may be used in conjunction with a vector. If a vector is used then the choice of vector is dependent upon the method that will be used to transform host cells as is well known to those skilled in the art. For example, a plasmid vector can be used. The skilled artisan is well aware of the genetic elements that must be present on the vector in order to successfully transform, select and propagate host cells comprising any of the isolated nucleic acid fragments of the invention. The skilled artisan will also recognize that different independent transformation events will result in different levels and patterns of expression (Jones et al., (1985) EMBO J. 4:2411-2418; De Almeida et al., (1989) Mol. Gen. Genetics 218:78-86), and thus that multiple events must be screened in order to obtain lines displaying the desired expression level and pattern. Such screening may be accomplished by Southern analysis of DNA, Northern analysis of mRNA expression, immunoblotting analysis of protein expression, or phenotypic analysis, among others. Vectors can be plasmids, viruses, bacteriophages, pro-viruses, phagemids, transposons, artificial chromosomes, and the like, that replicate autonomously or can integrate into a chromosome of a host cell. A vector can also be a naked RNA polynucleotide, a naked DNA polynucleotide, a polynucleotide composed of both DNA and RNA within the same strand, a poly-lysine-conjugated DNA or RNA, a peptide-conjugated DNA or RNA, a liposome-conjugated DNA, or the like, that is not autonomously replicating.

The term “expression”, as used herein, refers to the production of a functional end-product e.g., an mRNA or a protein (precursor or mature).

As used herein, the phrase “plant selectable or screenable marker” refers to a genetic marker functional in a plant cell. A selectable marker allows cells containing and expressing that marker to grow under conditions unfavorable to growth of cells not expressing that marker. A screenable marker facilitates identification of cells which express that marker.

As used herein, the term “inbred”, “inbred plant” is used in the context of the present invention. This also includes any single gene conversions of that inbred. The term single allele converted plant as used herein refers to those plants which are developed by a plant breeding technique called backcrossing wherein essentially all of the desired morphological and physiological characteristics of an inbred are recovered in addition to the single allele transferred into the inbred via the backcrossing technique.

As used herein, the term “sample” includes a sample from a plant, a plant part, a plant cell, or from a transmission vector, or a soil, water or air sample.

As used herein, the term “offspring” refers to any plant resulting as progeny from a vegetative or sexual reproduction from one or more parent plants or descendants thereof. For instance an offspring plant may be obtained by cloning or selfing of a parent plant or by crossing two parent plants and include selfings as well as the F1 or F2 or still further generations. An F1 is a first-generation offspring produced from parents at least one of which is used for the first time as donor of a trait, while offspring of second generation (F2) or subsequent generations (F3, F4, etc.) are specimens produced from selfings of F1's F2's etc. An F1 may thus be (and usually is) a hybrid resulting from a cross between two true breeding parents (true-breeding is homozygous for a trait), while an F2 may be (and usually is) an offspring resulting from self-pollination of said F1 hybrids.

As used herein, the term “cross”, “crossing”, “cross pollination” or “cross-breeding” refer to the process by which the pollen of one flower on one plant is applied (artificially or naturally) to the ovule (stigma) of a flower on another plant.

As used herein, the term “cultivar” refers to a variety, strain or race of plant that has been produced by horticultural or agronomic techniques and is not normally found in wild populations.

As used herein, the terms “dicotyledon” and “dicot” refer to a flowering plant having an embryo containing two seed halves or cotyledons. Dicotyledon plants at least include the Eudicot, Magnoliid, Amborella, Nymphaeales, Austrobaileyales, Chloranthales, and Ceratophyllum groups. Eudicots include these clades: Ranunculales, sabiales, Proteales, Trochodendrales, Buxales, and Core Eudicots (e.g., Berberidopsidales, Dilleniales, Gunnerales, Caryophyllales, Santalales, Saxifragales, Vitales, Rosids and Asterids). Non-limiting examples of dicotyledon plants include tobacco, tomato, pea, alfalfa, clover, bean, soybean, peanut, members of the Brassicaceae family (e.g., camelina, Canola, oilseed rape, etc.), amaranth, sunflower, sugarbeet, cotton, oaks, maples, roses, mints, squashes, daisies, nuts; cacti, violets and buttercups.

As used herein, the term “monocotyledon” or “monocot” refer to any of a subclass (Monocotyledoneae) of flowering plants having an embryo containing only one seed leaf and usually having parallel-veined leaves, flower parts in multiples of three, and no secondary growth in stems and roots. Non-limiting examples of monocotyledon plants include lilies, orchids, corn, rice, wheat, barley, sorghum, millets, oats, ryes, triticales, buckwheats, fonio, quinoa, grasses, such as tall fescue, goat grass, and Kentucky bluegrass; grains, such as wheat, oats and barley, irises, onions, palms.

As used herein, the term “nut” or “nut plant” refers to a plant producing dry fruit with one or more seeds in which the ovary wall becomes very hard (stony or woody) at maturity. Non-limiting examples of nuts include, Family Fagaceae (e.g., beech (Fagus), chestnut (Castanea), oak (Quercus), Tanoak (Lithocarpus)), Family Betulaceae (e.g., alder (Alnus), hazel, filbert (Corylus), and hornbeam), cuisine, almonds, pecans, walnuts, Brazil nut, Candlenut, cashew nut, gevuinanut, horse-chestnut, macadamia nut, Malabar chestnut, mongongo, peanut, pine nut, pistachio, et al.

As used herein, the term “gene” refers to any segment of DNA associated with a biological function. Thus, genes include, but are not limited to, coding sequences and/or the regulatory sequences required for their expression. Genes can also include nonexpressed DNA segments that, for example, form recognition sequences for other proteins. Genes can be obtained from a variety of sources, including cloning from a source of interest or synthesizing from known or predicted sequence information, and may include sequences designed to have desired parameters.

As used herein, the term “genotype” refers to the genetic makeup of an individual cell, cell culture, tissue, organism (e.g., a plant), or group of organisms.

As used herein, the term “hemizygous” refers to a cell, tissue or organism in which a gene is present only once in a genotype, as a gene in a haploid cell or organism, a sex-linked gene in the heterogametic sex, or a gene in a segment of chromosome in a diploid cell or organism where its partner segment has been deleted.

As used herein, the terms “heterologous polynucleotide” or a “heterologous nucleic acid” or an “exogenous DNA segment” refer to a polynucleotide, nucleic acid or DNA segment that originates from a source foreign to the particular host cell, or, if from the same source, is modified from its original form. Thus, a heterologous gene in a host cell includes a gene that is endogenous to the particular host cell, but has been modified. Thus, the terms refer to a DNA segment which is foreign or heterologous to the cell, or homologous to the cell but in a position within the host cell nucleic acid in which the element is not ordinarily found. Exogenous DNA segments are expressed to yield exogenous polypeptides.

As used herein, the term “heterologous trait” refers to a phenotype imparted to a transformed host cell or transgenic organism by an exogenous DNA segment, heterologous polynucleotide or heterologous nucleic acid.

As used herein, the term “heterozygote” refers to a diploid or polyploid individual cell or plant having different alleles (forms of a given gene) present at least at one locus.

As used herein, the term “heterozygous” refers to the presence of different alleles (forms of a given gene) at a particular gene locus.

As used herein, the terms “homolog” or “homologue” refer to a nucleic acid or peptide sequence which has a common origin and functions similarly to a nucleic acid or peptide sequence from another species.

As used herein, the term “homozygote” refers to an individual cell or plant having the same alleles at one or more loci.

As used herein, the term “homozygous” refers to the presence of identical alleles at one or more loci in homologous chromosomal segments.

As used herein, the term “hybrid” refers to any individual cell, tissue or plant resulting from a cross between parents that differ in one or more genes.

As used herein, the term “inbred” or “inbred line” refers to a relatively true-breeding strain.

As used herein, the term “line” is used broadly to include, but is not limited to, a group of plants vegetatively propagated from a single parent plant, via tissue culture techniques or a group of inbred plants which are genetically very similar due to descent from a common parent(s). A plant is said to “belong” to a particular line if it (a) is a primary transformant (T0) plant regenerated from material of that line; (b) has a pedigree comprised of a T0 plant of that line; or (c) is genetically very similar due to common ancestry (e.g., via inbreeding or selfing). In this context, the term “pedigree” denotes the lineage of a plant, e.g. in terms of the sexual crosses affected such that a gene or a combination of genes, in heterozygous (hemizygous) or homozygous condition, imparts a desired trait to the plant.

As used herein, the terms “mutant” or “mutation” refer to a gene, cell, or organism with an abnormal genetic constitution that may result in a variant phenotype. As used herein, the term “open pollination” refers to a plant population that is freely exposed to some gene flow, as opposed to a closed one in which there is an effective barrier to gene flow.

As used herein, the terms “open-pollinated population” or “open-pollinated variety” refer to plants normally capable of at least some cross-fertilization, selected to a standard, that may show variation but that also have one or more genotypic or phenotypic characteristics by which the population or the variety can be differentiated from others. A hybrid, which has no barriers to cross-pollination, is an open-pollinated population or an open-pollinated variety.

As used herein when discussing plants, the term “ovule” refers to the female gametophyte, whereas the term “pollen” means the male gametophyte.

As used herein, the term “phenotype” refers to the observable characters of an individual cell, cell culture, organism (e.g., a plant), or group of organisms which results from the interaction between that individual's genetic makeup (i.e., genotype) and the environment.

As used herein, the term “plant tissue” refers to any part of a plant. Examples of plant organs include, but are not limited to the leaf, stem, root, tuber, seed, branch, pubescence, nodule, leaf axil, flower, pollen, stamen, pistil, petal, peduncle, stalk, stigma, style, bract, fruit, trunk, carpel, sepal, anther, ovule, pedicel, needle, cone, rhizome, stolon, shoot, pericarp, endosperm, placenta, berry, stamen, and leaf sheath.

As used herein, the term “self-crossing”, “self pollinated” or “self-pollination” means the pollen of one flower on one plant is applied (artificially or naturally) to the ovule (stigma) of the same or a different flower on the same plant.

As used herein, the term “transformation” refers to the transfer of nucleic acid (i.e., a nucleotide polymer) into a cell. As used herein, the term “genetic transformation” refers to the transfer and incorporation of DNA, especially recombinant DNA, into a cell.

As used herein, the term “transformant” refers to a cell, tissue or organism that has undergone transformation. The original transformant is designated as “T0” or “T₀.” Selfing the T0 produces a first transformed generation designated as “T1” or “T₁.”

As used herein, the term “transgene” refers to a nucleic acid that is inserted into an organism, host cell or vector in a manner that ensures its function.

As used herein, the term “transgenic” refers to cells, cell cultures, organisms (e.g., plants), and progeny which have received a foreign or modified gene by one of the various methods of transformation, wherein the foreign or modified gene is from the same or different species than the species of the organism receiving the foreign or modified gene.

As used herein, the term “transposition event” refers to the movement of a transposon from a donor site to a target site.

As used herein, the term “variety” refers to a subdivision of a species, consisting of a group of individuals within the species that are distinct in form or function from other similar arrays of individuals.

As used herein, the term “vector”, “plasmid”, or “construct” refers broadly to any plasmid or virus encoding an exogenous nucleic acid. The term should also be construed to include non-plasmid and non-viral compounds which facilitate transfer of nucleic acid into virions or cells, such as, for example, polylysine compounds and the like. The vector may be a viral vector that is suitable as a delivery vehicle for delivery of the nucleic acid, or mutant thereof, to a cell, or the vector may be a non-viral vector which is suitable for the same purpose. Examples of viral and non-viral vectors for delivery of DNA to cells and tissues are well known in the art and are described, for example, in Ma et al. (1997, Proc. Natl. Acad. Sci. U.S.A. 94:12744-12746).

As used herein, the phrase “seed size” refers to the volume of the seed material itself, which is the space occupied by the constituents of the seed.

As used herein, the phrase “Test Weight” or “Grain Test Weight” is a determination of bulk density (mass/volume), measured for commerce under specific conditions defined in the U.S. by the USDA-FGIS. Test weight is a general indicator of grain quality and higher test weight normally means higher quality grain. Grain test weight in units of pounds per bushel specifies the weight of a “volume” bushel, which is 32 quarts (30,283 cubic centimeters) of grain. When grain is traded, samples are usually tested for quality, and test weight is one of the tests carried out. Test weights have been a part of U.S. grain grades since the United States Grain Standards Act was passed by Congress in 1916. U.S. grades for most grains specify test weight minimums for each grade level. For instance, the official minimum allowable test weight in the U.S. for No. 1 yellow corn is 56 lbs/bu and for No. 2 yellow corn is 54 lbs/bu (USDA-GIPSA, 1996). By law, a “weight” bushel of corn is exactly 56 pounds, a soybean bushel is 60 pounds, and a wheat bushel is 60 pounds, regardless of the test weight. The “weight” bushel is used for the basis of payment for grain, but price discounts are often tied to shipments of lower grade grain possessing low test weight.

As used herein, the phrase “Grain Apparent Density” refers to grain density determined in a fashion wherein the bulk density (mass/volume) of cereal seed is sometimes measured with the aid of a gas pycnometer, which typically uses helium and measures the volume of the sample. Grain kernels contain internal void spaces and intercellular spaces and are not completely porous to helium. Since the gas cannot reach all internal spaces, the volume of material comprising the kernel can be overestimated with gas pycnometry and a density lower than the “true density” of grain material is determined (Chang, C S (1988) Cereal Chem: 65:13-15).

As used herein, the phrase “Grain True Density” refers to the bulk density of grain, expressed as the quotient of mass divided by volume, whereby all void space not comprising solid materials of the seed has been eliminated before, or discounted in, determination of the volume used in the calculation (Chang, C S (1988) Cereal Chem: 65:13-15).

### Sucrose Isomerases

Sucrose isomerases (EC 5.4.99.11) refer to enzymes derived from microorganisms and insects that can produce isomaltulose (alpha-D-glucosylpyranosyl-1,6-D-fructofuranose, or 6-O-D-glucopyranosyl-D-fructose, also called palatinose) and/or trehalulose (alpha-D-glucosylpyranosyl-1,1-D-fructofuranose, or 6-O-D-glucopyranosyl-D-fructose) from the disaccharide sucrose, thus also as known as isomaltulose synthase, isomaltulose synthetase, sucrose alpha-glucosyltransferase, or trehalulose synthase. Such enzymes were previously isolated from several microbes including Protaminobacter rubrum (Nakajima, Y. (1988) J. Jpn. Soc. Starch Sci. 35, 131-139), Erwinia rhapontici (Cheetam, P. S. J. (1983) Biochem. J. 220. 213-220), Serratia plymnuthica (Veèroneése, T. and Perlot, P. (1998) Enzyme Microbiol. Technol., in press), Pseudomonas mesoacidophila (Nagai et al., (1994) Biosci. Biotech. Biochem. 58, 1789-1793), Klebsiella planticola (Ideno et al. (1991) The Annual Meeting of the Japan Society for Bioscience, Biotechnology and Agrochemistry, Kyoto, April 1991, pp. 284; Zhang et al., Appl Environ Microbiol. 2002 June; 68(6):2676-82), Enterobacter sp. (Cha et al., J Appl Microbiol. 2009 October; 107(4):1119-30. Epub 2009 Mar. 30), and insects, e.g., Bemisia argentifiulii (Salvucci, Comp Biochem Physiol B Biochem Mol. Biol. 2003 June; 135(2):385-95). More study on sucrose isomerases can be found in references 1-16, each of which is hereby incorporated by reference in its entirety.

Isomaltulose and trehalulose are commercially used since 1985 as non-cariogenic sucrose replacements and can be found in products for diabetics and people with prediabetic dispositions. They are resistant to invertases and are not metabolized by many microbes, including the predominant oral microflora, conferring an advantage for use in foods as an acariogenic sweetener. They are also attractive renewable starting materials for manufacture of biomaterials as petrochemical replacements (Lichtenthaler and Peters, 2004, C. R. Chim. 7:65-90). In addition, several other characteristics make isomaltulose advantageous over sucrose for some applications in the food industry: low glycemic index (useful for diabetics); selective promotion of growth of beneficial bifidobacteria among human intestinal microflora; greater stability of isomaltulose-containing foods and beverages; being less hygroscopic; and simple conversion into sugar alcohols which impacts other useful properties in food products.

Previously isolated sucrose isomerases from Protaminobacter rubrum (Weidenhagen & Lorenz 1957, Z. Zuckerindust. 7: 533-534), Serratia plymuthica (Fujii et al. 1983, Nippon Shokuhin Kogvo Gakkaishi 30: 339-344) or Erwinia rhapontici (Cheetham 1983, Biochem J. 220: 213-220) mainly produce isomaltulose (about 80%-90%); while the sucrose isomerases from the genus Klebsiella, Pseudomonas mesoacidophila MX-45 (Miyata et al. 1992, Biosci. Biotech. Biochem. 54: 1680-1681) and Agrobacterium radiobacter MX-232 (Nagai-Miyata et al. 1993, Biosci. Biotech. Biochem. 57: 2049-2053) mainly produce trehalulose.

The inventors of the present invention unexpectedly observed that expressing a sucrose isomerase in a plant leads to dramatic increase of seed weight, seed number and/or seed size. Thus, the present invention in one aspect provides methods of increasing seed weight, seed number and/or size in a plant by expressing a sucrose isomerase, biologically active variants, or fragments thereof. Without wishing to be bound by theory, expression of a sucrose isomerase of the present invention may increase seed weight by increasing the density of a seed without significantly affecting the size of the seed; by increasing the size of a seed without significantly affecting the seed density; or by increasing both the seed density and the seed size. For example, an increased seed weight may be due to (1) the seed becomes larger in volume with no change in true density and, in turn, is heavier; and/or (2) the true density of the seed increases, meaning there is no change in volume but an increase of weight per volume (g/cc). Thus, in some embodiments, the “test weight” of a seed is increased. For corn, and other cereal crops, grain “test weight” is a grading factor of significant economic importance, see, e.g., Glossary of Crop Science Terms (July 1992) Crop Science Society of America, Madison, Wis., page 35. Thus, the methods and compositions of the present invention provide seed with increased seed size, increased seed volume, increased seed weight, increased seed test weight, increased grain apparent density, and/or increased grain true density when compared to the seed produced by the corresponding wild type plants (i.e., the same or similar genotype without a nucleic acid sequence encoding a sucrose isomerase, or a biologically active variant, or fragment thereof, operably linked to a stem specific or stem preferred promoter).

Sucrose isomerases that can be used in the present invention include, but are not limited to any sucrose isomerase that have been or could be isolated and biologically active variants or fragments thereof. For example, sucrose isomerase can be selected from the group consisting of sucrose isomerase from Raoultella planticola (Accession No. AAP57085.1), sucrose isomerase from Erwinia rhapontici (Accession No. AAP57084.1), sucrose isomerase from Pantoea dispersa, (Accession No. AAP57083.1), sucrose isomerase from Pectobacterium atrosepticumn SCR11043 (Accession No. YP_{—}049947.1), sucrose isomerase from Enterobacter sp. FMB-1 (Accession No. ACF42098.1), sucrose isomerase from Pectobacterium atrosepticuum SCR11043 (Accession No. CAG74753.1), sucrose isomerase from Leucaena leucocephala (Accession No. ABB01680.1), sucrose isomerase from Erwinia pyrifoliae Ep1/96 (Accession No. YP_{—}002648731.1), sucrose isomerase from Erwinia amylovora ATCC 49946 (Accession No. YP_{—}003538902.1), sucrose isomerase from Erwinia tasmaniensis Et1/99 (Accession No. YP_{—}001907593.1), sucrose isomerase from Azotobacter vinelandii DJ (Accession No. YP_{—}002798053.1), sucrose isomerase from Erwinia rhapontici (Accession No. AAK28735.1), sucrose isomerase from Pectobacterium carotovorum subsp. brasiliensis PBR1692, (Accession No. ZP_{—}03825380.1), sucrose isomerase precursor from Pseudornmonas mesoacidophila (Accession No. ABC33903.1), trehalulose synthase from Pseudomonas mesoacidophila (Accession No. ACO005018.1), alpha-glucosyltransferase from Klebsiella sp. NK33-98-8 (Accession No. AAM96902.1), MutB protein (derived from Pseudomonas mesoacidophila MX-45, SEQ ID NO: 2), and UQ68J protein (derived from Pantoea dispersa, SEQ ID NO: 8). All the sequences mentioned herein are incorporated by reference in their entireties.

General texts which describe molecular biological techniques, which are applicable to the present invention, such as cloning, mutation, cell culture and the like, include Berger and Kimmel, Guide to Molecular Cloning Techniques. Methods in Enzymology, Vol. 152 Academic Press, Inc., San Diego, Calif. (“Berger”); Sambrook et al., Molecular Cloning—A Laboratory Manual (3rd Ed.), Vol. 1-3, Cold Spring Harbor Laboratory, Cold Spring Harbor. N.Y., 2000 (“Sambrook”) and Current Protocols in Molecular Biology, F. M. Ausubel et al., eds., Current Protocols, a joint venture between Greene Publishing Associates, Inc. and John Wiley & Sons, Inc., (“Ausubel”). These texts describe mutagenesis, the use of vectors, promoters and many other relevant topics related to, e.g., the cloning and mutating of sucrose isomerase. Thus, the invention also encompasses using known methods of protein engineering and recombinant DNA technology to improve or alter the characteristics of the sucrose isomerases expressed in plants. Various types of mutagenesis can be used to produce and/or isolate variant nucleic acids that encode for protein molecules and/or to further modify/mutate the sucrose isomerases. They include but are not limited to site-directed, random point mutagenesis, homologous recombination (DNA shuffling), mutagenesis using uracil containing templates, oligonucleotide-directed mutagenesis, phosphorothioate-modified DNA mutagenesis, mutagenesis using gapped duplex DNA or the like. Additional suitable methods include point mismatch repair, mutagenesis using repair-deficient host strains, restriction-selection and restriction-purification, deletion mutagenesis, mutagenesis by total gene synthesis, double-strand break repair, and the like. Mutagenesis, e.g., involving chimeric constructs, is also included in the present invention. In some embodiments, mutagenesis can be guided by known information of the naturally occurring molecule or altered or mutated naturally occurring molecule, e.g., sequence, sequence comparisons, physical properties, crystal structure or the like. The invention further comprises sucrose isomerase variants which show substantial biological activity, e.g., able to convert sucrose to isomaltulose and/or trehalulose. Such variants include deletions, insertions, inversions, repeats, and substitutions selected according to general rules known in the art so as have little effect on activity.

In some embodiments, variant sucrose isomerase proteins differ in amino acid sequence from the sequences described herein but that share at least 65% amino acid sequence identity with such enzyme sequences, but still maintain substantial biological activity. In other embodiments, other variants will share at least 70%, at least 75%, at least 80%, at least 85%, at least 90%, at least 91%, at least 92%, at least 93%, at least 94%, at least 95%, at least 96%, at least 97%, at least 98%, or at least 99% amino acid sequence identity. Manipulation of corresponding gene (including +/−upstream and downstream flanking regions) and ORF nucleotide sequences using standard procedures (e.g., site-directed mutagenesis or PCR) can be used to produce such variants. The simplest modifications involve the substitution of one or more amino acids for amino acids having similar biochemical properties. Said amino acid substitutions may be conservative or non-conservative. These so-called conservative substitutions are likely to have minimal impact on the activity of the resultant protein. The non-conservative substitution may or may not reduce the activity of the resultant protein. So long as the resultant protein still maintains a practically useful level of activity to increase seed weight, seed number and/or seed size in a plant, it can be used in the present invention.

In some embodiments, the function of a sucrose isomerase can be maintained in variants if amino acid substitutions are introduced in regions outside of the conserved domains of the protein, where amino acid substitutions are less likely to affect protein function. As used herein, the term “domain” generally refers to a portion of a protein or nucleic acid that is structurally and/or functionally distinct from another portion of the protein or nucleic acid.

In another embodiment, more substantial changes in the sucrose isomerase enzyme function or other protein features may be obtained by selecting amino acid substitutions that are less conservative than conservative substitutions. In one specific, non-limiting, embodiment, such changes include changing residues that differ more significantly in their effect on maintaining polypeptide backbone structure (e.g., sheet or helical conformation) near the substitution, charge or hydrophobicity of the molecule at the target site, or bulk of a specific side chain. The following specific, non-limiting, examples are generally expected to produce the greatest changes in protein properties: (a) a hydrophilic residue (e.g., seryl or threonyl) is substituted for (or by) a hydrophobic residue (e.g., leucyl, isoleucyl, phenylalanyl, valyl or alanyl); (b) a cysteine or proline is substituted for (or by) any other residue; (c) a residue having an electropositive side chain (e.g., lysyl, arginyl, or histidyl) is substituted for (or by) an electronegative residue (e.g., glutamyl or aspartyl); or (d) a residue having a bulky side chain (e.g., phenylalanine) is substituted for (or by) one lacking a side chain (e.g., glycine).

Variant sucrose isomerase-encoding sequences may be produced by standard DNA mutagenesis techniques. In one specific, non-limiting, embodiment, M13 primer mutagenesis is performed. Details of these techniques are provided in Sambrook et al. (In Molecular Cloning: A Laboratory Manual, Cold Spring Harbor Laboratory Press, New York, 1989), Ch. 15. By the use of such techniques, variants may be created that differ from the isomerases sequences. DNA molecules and nucleotide sequences that are derivatives of those specifically disclosed herein, and which differ from those disclosed by the deletion, addition, or substitution of nucleotides while still encoding a protein having the biological activity of the prototype enzyme. The resulting product gene can be cloned as a DNA insert into a vector. In many, but not all, common embodiments, the vectors of the present invention are plasmids or bacmids.

Conservative amino acid substitutions are those substitutions that, when made, least interfere with the properties of the original protein, that is, the structure and especially the function of the protein is conserved and not significantly changed by such substitutions. Conservative substitutions generally maintain (a) the structure of the polypeptide backbone in the area of the substitution, for example, as a sheet or helical conformation, (b) the charge or hydrophobicity of the molecule at the target site, or (c) the bulk of the side chain. Further information about conservative substitutions can be found, for instance, in Ben Bassat et al. (J. Bacteriol., 169:751-757, 1987), O'Regan et al. (Gene, 77:237-251, 1989), Sahin-Toth et al. (Protein Sci., 3:240-247, 1994), Hochuli et al. (Bio/Technology, 6:1321-1325, 1988) and in widely used textbooks of genetics and molecular biology. The Blosum matrices are commonly used for determining the relatedness of polypeptide sequences. The Blosum matrices were created using a large database of trusted alignments (the BLOCKS database), in which pairwise sequence alignments related by less than some threshold percentage identity were counted (Henikoff et al., Proc. Natl. Acad. Sci. USA, 89:10915-10919, 1992). A threshold of 90% identity was used for the highly conserved target frequencies of the BLOSUM90 matrix. A threshold of 65% identity was used for the BLOSUM65 matrix. Scores of zero and above in the Blosum matrices are considered “conservative substitutions” at the percentage identity selected. The following table shows non-limiting exemplary conservative amino acid substitutions.

TABLE 1
Conservation Amino Acid Substitution

| | Very Highly - | | |
|---|---|---|---|
| Original | Conserved | Highly Conserved Substitutions | Conserved Substitutions |
| Residue | Substitutions | (from the Blosum90 Matrix) | (from the Blosum65 Matrix) |
| Ala | Ser | Gly, Ser, Thr | Cys, Gly, Ser, Thr, Val |
| Arg | Lys | Gln, His, Lys | Asn, Gln, Glu, His, Lys |
| Asn | Gln; His | Asp, Gln, His, Lys, Ser, Thr | Arg, Asp, Gln, Glu, His, Lys, Ser, Thr |
| Asp | Glu | Asn, Glu | Asn, Gln, Glu, Ser |
| Cys | Ser | None | Ala |
| Gln | Asn | Arg, Asn, Glu, His, Lys, Met | Arg, Asn, Asp, Glu, His, Lys, Met, Ser |
| Glu | Asp | Asp, Gln, Lys | Arg, Asn, Asp, Gln, His, Lys, Ser |
| Gly | Pro | Ala | Ala, Ser |
| His | Asn; Gln | Arg, Asn, Gln, Tyr | Arg, Asn, Gln, Glu, Tyr |
| Ile | Leu; Val | Leu, Met, Val | Leu, Met, Phe, Val |
| Leu | Ile; Val | Ile, Met, Phe, Val | Ile, Met, Phe, Val |
| Lys | Arg; Gln; Glu | Arg, Asn, Gln, Glu | Arg, Asn, Gln, Glu, Ser, |
| Met | Leu; Ile | Gln, Ile, Leu, Val | Gln, Ile, Leu, Phe, Val |
| Phe | Met; Leu; Tyr | Leu, Trp, Tyr | Ile, Leu, Met, Trp, Tyr |
| Ser | Thr | Ala, Asn, Thr | Ala, Asn, Asp, Gln, Glu, Gly, Lys, Thr |
| Thr | Ser | Ala, Asn, Ser | Ala, Asn, Ser, Val |
| Trp | Tyr | Phe, Tyr | Phe, Tyr |
| Tyr | Trp; Phe | His, Phe, Trp | His, Phe, Trp |
| Val | Ile; Leu | Ile, Leu, Met | Ala, Ile, Leu, Met, Thr |

In some examples, variants can have no more than 3, 5, 10, 15, 20, 25, 30, 40, 50, or 100 conservative amino acid changes (such as very highly conserved or highly conserved amino acid substitutions). In other examples, one or several hydrophobic residues (such as Leu, Ile, Val, Met, Phe, or Trp) in a variant sequence can be replaced with a different hydrophobic residue (such as Leu, Ile, Val, Met, Phe, or Trp) to create a variant functionally similar to any of the sucrose isomerases as mentioned herein. For example, the present invention provides SEQ ID NO: 13 which is a modified UQ68J protein with 30 amino acids substitution. This modified UQ68J protein shares 95% identity to the wild type UQ68J protein, and has similar enzyme activity of wild type UQ68J protein.

In some embodiments, variants may differ from the sucrose isomerase sequences described herein by alteration of the coding region to fit the codon usage bias of the particular organism into which the molecule is to be introduced. In other embodiments, the coding region may be altered by taking advantage of the degeneracy of the genetic code to alter the coding sequence such that, while the nucleotide sequence is substantially altered, it nevertheless encodes a protein having an amino acid sequence substantially similar to the sucrose isomerase sequences described herein. For example, because of the degeneracy of the genetic code, four nucleotide codon triplets (GCT, GCG, GCC and GCA) code for alanine. The coding sequence of any specific alanine residue within a sucrose isomerase, therefore, could be changed to any of these alternative codons without affecting the amino acid composition or characteristics of the encoded protein. Based upon the degeneracy of the genetic code, variant DNA molecules may be derived from the nucleic acid sequences disclosed herein using standard DNA mutagenesis techniques, as described herein, or by synthesis of DNA sequences.

Based on the polynucleotide sequences of sucrose isomerase genes and polypeptide sequences of sucrose isomerase proteins described in the invention, one skilled in the art will be able to design variant nucleic acid sequences encoding a protein having similar function of sucrose isomerase by virtue of the degeneracy of the genetic code. One skilled in the art will also be able to isolate variant nucleic acid sequences encoding a protein having similar function of sucrose isomerase from a species other than those mentioned herein. In some embodiments, homologous genes from other species can be cloned by the classical approach, wherein it involves the purification of the target protein, obtaining amino acid sequences from peptides generated by proteolytic digestion and reverse translation of the peptides. The derived DNA sequence, which is bound to be ambiguous due to the degeneracy of the genetic code, can then be employed for the construction of probes to screen a gene library. In some embodiments, PCR methods can be used to isolate fragments of homologous genes containing at least two blocks of conserved amino acids. The amino acid sequence of a conserved region is reverse translated and a mixture of oligonucleotides is synthesized representing all possible DNA sequences coding for that particular amino acid sequence. Two such degenerate primer mixtures derived from appropriately spaced conserved blocks are employed in a PCR reaction. The PCR products are then, usually after enrichment for the expected fragment length, cloned and sequenced. In some embodiments, a homologous sucrose isomerase gene can be isolated based on hybridization of two nucleic acid molecules under stringent conditions. More detailed methods of cloning homologous genes based on a known gene is described in “Gene Cloning and DNA Analysis: An Introduction”, (Publisher: John Wiley and Sons, 2010, ISBN 1405181737, 9781405181730), and “Gene cloning: principles and applications” (Publisher: Nelson Thomes, 2006).

In some embodiments, the invention provides sucrose isomerases that may comprise, mutations containing alterations that produce silent substitutions, additions, or deletions, but do not alter the properties or activities of the encoded protein or how the proteins are made. Nucleotide variants can be produced for a variety of reasons, e.g., to optimize codon expression for a particular host (e.g., change codons in microbes to those preferred by plant cells).

In some embodiments, the invention provides chimeric proteins, wherein the chimeric proteins comprise polypeptide of a sucrose isomerase, or comprise variants and/or fragments of a sucrose isomerase, which is fused to one or more other polypeptides. Polynucleotides that encode such chimeric proteins can be cloned into an expression vector that can be expressed in a plant cell. In some embodiments, the polynucleotide encoding sucrose isomerase is operably linked to one or more DNA encoding a signal peptide which targets the fusion polypeptide produced therefrom to an organelle of the plant, wherein the seed weight, seed number and/or seed size of the plant are increased. In some embodiments, said organelle of the plant is a storage compartment and/or transport compartment. In some embodiments, said storage compartment is the vacuole. The vacuole-targeting signal peptides can be either fused to the N-terminal or to the C-terminal of the sucrose isomerase, variants and fragments thereof.

In some embodiments, the sucrose isomerase is any biologically active chimeric sucrose isomerase designed in silico using gene shuffling and/or directed molecular evolution.

Gene shuffling (a.k.a. DNA shuffling, or sexual PCR), is a way to rapidly propagate beneficial mutations in a directed evolution experiment. Gene shuffling provides new ways to improve the functionality of genes, thus improving traits and creating higher-performing products. Non-limiting exemplary methods of using gene shuffling to produce chimeric genes are described in U.S. Pat. Nos. 6,521,453, 6,423,542, 6,479,652, 6,368,861, 6,500,639, and U.S. Patent Application Publication Nos. 20060141626, 20040191772, 20040053267, 20030104417, and 20080171668, each of which is herein incorporated by reference in its entirety.

Directed evolution (DE) has in recent years emerged as an effective technique for generating and selecting proteins with a variety of uses. The starting point is usually a library containing proteins that already possess the desired function to some extent, although randomly generated proteins have also been used. Through a series of iterative steps, or ‘generations’, during each of which the proteins are diversified and then screened, the protein library is ‘evolved’ towards better performance. Several evoluted proteins have been described previously (see, Chemy and Fidantsef, 2003; Sylvestre et al., 2006; Yun et al. 2006; Chautard et al., 2007; Joyce, 1994; and Piatesi et al., 2006). Non-limiting exemplary methods of directed molecular evolution are described in Jackson et al. (Directed Evolution of Enzymes, Comprehensive Natural Products II, 2010, Chapter 9.20, Pages 723-749), Rubin-Pitel et al. (Directed Evolution Tools in Bioproduct and Bioprocess Development Bioprocessing for Value-Added Products from Renewable Resources, 2007, Pages 49-72), Reetz (Directed evolution of selective enzymes and hybrid catalysts, Tetrahedron. Volume 58, Issue 32, 5 Aug. 2002, Pages 6595-6602), Datamonitor (Datamonitor reports, Directed molecular evolution: product life cycle management for biologics, 2006, Electronic books), Brakmann and Johnsson (Directed molecular evolution of proteins: or how to improve enzymes for biocatalysis, Publisher: Wiley-VCH, 2002, ISBN 3527304231, 9783527304233), Davies (Directed molecular evolution by gene conversion, Publisher University of Bath, 2001), Arnold and Georgiou (Directed enzyme evolution: screening and selection methods, Publisher: Humana Press, 2003, ISBN 158829286X, 9781588292865), and directed evolution library creation: methods and protocols, Publisher Humana Press, 1984, ISBN 1588292851, 9781588292858), each of which is incorporated by reference in its entirety.

Computer-assistant design of directed evolution can be utilized, following the non-limiting exemplary strategies and methods as described in Wedge et al. (In silico modeling of directed evolution: Implications for experimental design and stepwise evolution, Journal of Theoretical Biology, Volume 257, Issue 1, 7 Mar. 2009, Pages 131-141), Knowles (Closed-loop evolutionary multiobjective optimization, IEEE Computational Intelligence Magazine 4 (3), art. no. 5190940, pp. 77-91), Francois and Hakim (Design of genetic networks with specified functions by evolution in silico, PNAS Jan. 13, 2004 vol. 101 no. 2 580-585), Sole et al. (Synthetic protocell biology: from reproduction to computation, Phil. Trans. R. Soc. B. 2007 362 (1486) 1727-1739), and Marguet et al. (Biology by design: reduction and synthesis of cellular components and behavior, J R Soc Interface 2007 4 (15) 607-623), each of which is incorporated by reference in its entirety.

Vacuole-targeting signal peptides are well known to one skilled in the art. Non-limiting examples of vacuole-targeting signal peptides (a.k.a. sequence-specific vacuolar sorting signal peptides) include, 16 amino acids of prosporamin storage protein in sweet potato tubers (Nakamura et al., J Exp Bot 44 (Suppl): 331-338 (1993), a.k.a the N′terminal propeptide (NTPP) of sporamin, SEQ ID NO: 21), 21 amino acids of barley proaleurain (Holwerda et al., J Exp Bot 44 (Suppl): 321-339, 1993, SEQ ID NO: 26, N-terminal), 7 amino acids of tobacco chitinase A (Neuhaus et al. Proc Natl Acad Sci USA, 88: 10362-10366, 1991, SEQ ID NO: 27, C-terminal), signal peptide of barley lectin (Dombrowski et al., Plant Cell 5: 587-596, 1993), signal peptide of tobacco glucanase (Melchers et al., Plant Mol Biol 21: 583-593, 1993), signal peptide of tobacco osmotin (Sticher et al., Planta 188: 559-565, 1992), signal peptide of 2S albumin storage proteins from Brazil nut (Saalbach et al., Plant Cell 3: 695-708, 1991) and pea (Higgins J Biol Chem 261: 11124-11130, 1986). More vacuole-targeting signal peptides have been described in Neuhaus et al. (Plant Molecular Biology 38: 127-144, 1998), De et al. (Plant cell vacuoles: an introduction, Publisher: CSIRO Publishing, 2000, ISBN 0643062548, 9780643062542), Jolliffe et al. (Biochemical Society Transactions (2005) 33:1016-1018), Nakamura et al. (Plant Physiol. (1993), 101:1-5), Hunt et al. (Plant Physiol. (1991) 96, 18-25), Marty (The Plant Cell, Vol. 11, 587-599, April 1999), and Holwerda et al. (The Plant Cell, Vol. 4, 307-318, March 1992), each of the references mentioned herein is incorporated by reference in its entirety. Thus, any of the vacuole-targeting signal peptide mentioned herein can be fused to sucrose isomerase, variants or fragments thereof to make chimeric protein, which will be sorted to vacuole when expressed in plant cells.

The sucrose isomerases in the present invention can originate from microbes or insects. To express these proteins in plants at high yield level and have them correctly transported to vacuole, post-translation modification of gene product may be involved. Particularly, post-translation modification in the endoplasmic reticulum (ER) may be important, since correct translocation to the ER of storage proteins as well as other plant proteins, requires an amino-terminal transit peptide that is cotranslationally removed (Chrispeels, 1984, Philos. Trans. R. SOC. Lond. B Biol. Sci. 304:309-322). The oxidizing environment of the ER, the lack of proteases and the abundance of molecular chaperones are all important factors for correct protein folding and assembly. Also, protein glycosylation occurs only in the ER system and this modification is required for the correct function of many proteins. Thus, the chimeric protein as described herein can further contain ER signal peptide. Such ER signal peptide sequence can direct ribosomes with newly synthesized sucrose isomerase to the ER membrane. After cleavage of the ER signal peptide, the matured protein in the ER lumen will be secreted to the vacuole. Transportation from ER to vacuole can occur through at least several pathways, including a) Amino-terminal propeptide (NTPP) pathway; b) Carboxy-terminal propeptides (CTPP); c) ER-to-vacuole pathway; d) ER-to-PAC-to-vacuole pathway; e) Secretion pathway; f) CCV endocytosis; and g) Receptor-mediated endocytosis (Surpin and Raikhel, 2004, Nature Reviews/Molecular Cell Biology 5:100-109). The ER signal peptide can be fused to the N-terminal of the chimeric protein comprising vacuole-targeting signal peptide and sucrose isomerase. The ER signal peptide can be any one of the ER signal peptides identified in plant proteins, e.g., in storage proteins. Non-limiting examples of ER signal peptides include, ER signal peptide from fava bean lectin, barley hordein, sweet potato sporamin A (SEQ ID NO: 21), tomato polygalacturonase, wheat high molecular weight glutenin, soybean protein VSPαS, chitinase (Gnanasambandam et al., Plant Biotechnology Journal, Volume 5 Issue 2, Pages 290-296), oleosin, patatin (Bevan et al., 1986, Nucleic Acids Res. 41, 4625-4638), 2s alhumin of Bertbolletia excelsa H.B.K. (Saalbach et al., Plant Physiol. (1996) 11 2: 975-985), proteinase inhibitor II (Herbers et al., 1995. Bio/Technology 13, 63-66.), and ER retention signal (KDEL or HDEL), each of the references mentioned herein is incorporated by reference in its entirety.

### Expression Vectors

The present invention provides expression vectors comprising nucleic acid sequences encoding chimeric sucrose isomerase. The backbone of the expression vectors can be any expression vectors suitable for producing transgenic plant, which are well known in the art. In some embodiments, the expression vector is suitable for expressing transgene in monocot plants, e.g., in cereal crops, such as maize, rice, wheat, barley, sorghum, millets, oats, ryes, triticales, buckwheats, fonio, quinoa and oil palm et al. In another embodiment, the expression vector is suitable for expressing transgene in dicot plants, such as beans, soybeans, peanuts, nuts, members of the Brassicaceae family (Camelina, oilseed rape, Canola, etc.), amaranth, cotton, peas, tomatoes, sugarbeet, and sunflower.

In some embodiments, the expression vector is an Agrobacterium binary vector (see, Karimi et al., Plant Physiol 145: 1183-1191; Komari et al., Methods Mol Biol 343: 15-42; Bevan M W (1984) Nucleic Acids Res 12: 1811-1821; Becker (1992), Plant Mol Biol 20: 1195-1197; Datla et al., (1992), Gene 122: 383-384; Hajdukiewicz (1994) Plant Mol Biol 25:989-994; Xiang (1999), Plant Mol Biol 40: 711-717; Chen et al., (2003) Mol Breed 11: 287-293; Weigel et al., (2000) Plant Physiol 122: 1003-1013). In another embodiment, the expression vector is a co-integrated vector (also called hybrid Ti plasmids). More expression vectors and methods of using them can be found in U.S. Pat. Nos. 4,940,838, 5,464,763, 5,149,645, 5,501,967, 6,265,638, 4,693,976, 5,635,381, 5,731,179, 5,693,512, 6,162,965, 5,693,512, 5,981,840, 6,420,630, 6,919,494, 6,329,571, 6,215,051, 6,369,298, 5,169,770, 5,376,543, 5,416,011, 5,569,834, 5,824,877, 5,959,179, 5,563,055, and 5,968,830. Each of the references mentioned herein is incorporated by reference in its entirety.

The nucleic acid sequence encoding chimeric sucrose isomerase is operably linked to a nucleic acid sequence of a plant promoter. Generally speaking, a plant promoter of the present invention can be either a constitutive promoter or a non-constitutive promoter, so long as the expression of a sucrose isomerase driven by the plant promoter can lead to increased seed weight, seed number and/or size. In some embodiments, the promoter is a tissue specific or tissue preferred promoter. In some embodiments, the tissue specific or tissue preferred promoters of the present invention useful for expressing sucrose isomerase in plant are stem specific and/or stem preferred promoters. A stem specific or a stem preferred promoter can be, for example, a stalk specific or stalk preferred promoter, a vascular specific or vascular specific promoter, a xylem specific or xylem preferred promoter, a phloem specific or phloem preferred promoter, or a xylem/phloem specific or a xylem/phloem preferred promoter. The stem specific and/or stem preferred promoters can be the promoters of cellulose synthesis catalytic complex involved in the synthesis of cellulose during plant secondary cell wall formation in the plant vascular tissue (stem tissue), such as Arabidopsis thaliana genes AtCesA4, AtCesA7, and AtCesA8; Zea mays genes ZmCesA 10, ZmCesA 11, and ZmCesA 12; and the Oryza sativa orthologs of ZmCesA 10, ZmCesA 11, and ZmCesA 12; rice ENOD40 promoter (see Kouchi et al., Plant J. 1999 April, 18(2):121-9; SEQ ID NO: 22), o-methyl transferase (OMT) promoter (e.g., sugarcane o-methyl transferase promoter, homologs, orthologs, or functional variants thereof, see Damaj et al., Planta. 2010 May, 231(6):1439-58; SEQ ID NO: 23), or jasmonate-inducible protein promoter, JAS (e.g., sugarcane JAS promoter, homologs, orthologs, or functional variants thereof, SEQ ID NO: 24). Also, any promoter homologous to (or having a high sequence identity to) any of the promoters mentioned and also exhibiting stem specific expression, as defined herein, can be used.

In some embodiments, the sucrose isomerase gene is under the control of a promoter associated with an early nodulin gene, homologs, orthologs, variants, functional gragment thereof. In some embodiments, the early nodulin gene is an ENOD40 gene. Examples of early nodulin genes have been disclosed previously, e.g., in Kouchi et al. (Plant J. 1999 April, 18(2):121-129) and Larsen (2003. Molecular cloning and characterization of a cDNA encoding a ryegrass (Lolium perenne) ENOD40 homologue, J. Plant Physiol. 160 (6), 675-687), including but are not limited to, Glycine max ENOD40a (GenBank ID NO. X69155); GmENOD40b (GenBank ID NO. X69154); Phaseolus vulgaris ENOD40 (GenBank ID NO. X86441); Lotus japonicus ENOD40 (H. Kouchi, unpublished data); Sesbania rostrata ENOD40 (GenBank ID NO. Y12714); Pisum sativum ENOD40 (GenBank ID NO. X81064); Vicia sativa ENOD40 (GenBank ID NO. X83683); Medicago sativa ENOD40 (GenBank ID NO. X80263); Medicago truncatula ENOD40 (GenBank ID NO. X80264); Trifolium repens ENOD40 (GenBank ID NO. AJ000268); Nicotiana tabacum ENOD40 (GenBank ID NO. X98716); Oryza sativa ENOD40 (GenBank ID NO. AB024054); Oryza brachyantha ENOD40 (GenBank ID NO. AB024055); Zea mays ENOD40 (GenBank ID NO. AI001271); Lolium perenne ENOD40 (GenBank ID NO. AF538351). Lolium perenne ENOD40 and Hordeum vulgare ENOD40 described in Larsen (2003). All references and GenBank ID Nos with associated sequences mentioned above are incorporated by reference in their entireties for all purposes.

In some embodiments, the sucrose isomerase gene is under the control of a promoter associated with an O-methyltransferase (OMT) gene, homologs, orthologs, variants, functional gragment thereof. Examples of OMT genes have been disclosed previously, e.g., in Damaj et al (2010), Sugarcane DIRIGENT and O-METHYLTRANSFERASE promoters confer stem-regulated gene expression in diverse monocots, Planta 231:1439-1458 (attached). They are also described in U.S. Pat. No. 7,323,622 B2, including but are not limited to. Saccharum spp. Hybrid (sugarcane hybrid) O-methyltransferase-like protein (SHOMT, GenBank ID NO. GU062719), Medicago truncatula (barrel clover) hydroxyisoflavanone 4-O-methyltransferase (MtHI4′OMT, GenBank ID NO. AY942158), Zea mays (maize) O-methyltransferase ZRP4 (ZmZRP4-2, GenBank ID NO. NM_{—}001155649), Z. mays O-methyltransferase (newly annotated as caffeic acid-3-O-methyltransferase, ZmOMT or ZmCOMT, GenBank ID NO. M73235), Arabidopsis thaliana (Arabidopsis) O-methyltransferase 1 (AtOMT1, GenBank ID NO. NM_{—}124796), Populus tremnuloides (quaking aspen) caffeic acid/5-hydroxyferulic acid O-methyltransferase (Ptrbi-OMT, GenBank ID NO. U13171), S. officinarum (sugarcane) caffeic acid 3-O-methyltransferase (SoCOMT, GenBank ID NO. AJ231133), M. sativa (alfalfa) caffeic acid/5-hydroxyferulic acid 3/5-O-methyltransferase (MsCOMT, GenBank ID NO. M63853). Homologs, orthologs of OMT genes include, but are not limited to genes associated with Sorghum bicolor hypothetical protein (ACCESSION XM_{—}002451916, similar to O-methyltransferase ZRP4), Sorghum bicolor hypothetical protein (ACCESSION XM_{—}002441335, similar to O-methyltransferase ZRP4), Sorghum bicolor hypothetical protein (ACCESSION XM_{—}002441331, similar to O-methyltransferase ZRP4), Sorghum bicolor hypothetical protein (ACCESSION XM_{—}002441334, similar to O-methyltransferase ZRP4), Sorghum bicolor hypothetical protein (ACCESSION XM_{—}002450280. similar to O-methyltransferase ZRP4), Sorghum bicolor hypothetical protein (ACCESSION XM_{—}002441327, similar to O-methyltransferase ZRP4), Zea mays LOC100283159 (umc2690, ACCESSION NM_{—}001156061. O-methyltransferase ZRP4; p-umc2690), Oryza sativa Japonica Group Os05g0515500 (Os05g0515500, ACCESSION NM_{—}001062574, similar to O-methyltransferase ZRP4 (EC 2.1.1.-)(OMT); Oryza sativa Japonica Group Os12g0441300 (Os12g0441300, ACCESSION NM_{—}001073230, similar to Flavonoid 4′-O-methyltransferase); Oryza sativa Japonica Group Os09g0344500 (Os09g0344500, ACCESSION NM_{—}001069466, similar to O-methyltransferase ZRP4 (EC 2.1.1.-)(OMT); Oryza sativa Japonica Group Os06g0281300 (Os06g0281300, ACCESSION NM_{—}001187785, similar to O-methyltransferase ZRP4 (EC 2.1.1.-)(OMT); Sorghum bicolor hypothetical protein (ACCESSION XM_{—}002438833, similar to O-methyltransferase ZRP4); Sorghum bicolor hypothetical protein (ACCESSION XM_{—}002438832. similar to O-methyltransferase ZRP4); Sorghum bicolor hypothetical protein (ACCESSION XM_{—}002437407. similar to O-methyltransferase ZRP4); Sorghum bicolor hypothetical protein, ACCESSION XM_{—}002447380, similar to O-methyltransferase ZRP4); Sorghum bicolor hypothetical protein (ACCESSION XM_{—}002449512, similar to O-methyltransferase ZRP4,); Zea mays O-methyltransferase ZRP4 (ACCESSION NM_{—}001165490); Zea mays O-methyltransferase ZRP4 (ACCESSION NM_{—}001155121), Brachypodium distachyon O-methyltransferase ZRP4-like, transcript variant 3 (LOC100833262) (ACCESSION XM_{—}003568061); Zea mays clone 689435 O-methyltransferase ZRP4 (ACCESSION EU976253); Zea mays clone 236658 O-methyltransferase ZRP4 mRNA (ACCESSION EU961576); Zea mays LOC100281319 (TIDP3344, ACCESSION NM_{—}001154237. O-methyltransferase ZRP4); Brachypodium distachyon O-methyltransferase ZRP4-like, transcript variant 1 (LOC100833262 ACCESSION XM_{—}003568059); Brachypodium distachyon O-methyltransferase ZRP4-like, transcript variant 2 (LOC100833262, ACCESSION XM_{—}003568060); Brachypodium distachyon O-methyltransferase ZRP4-like, transcript variant 4 (LOC100833262, ACCESSION XM_{—}003568062); Sorghum bicolor O-methyltransferase 3 (OMT3, ACCESSION EF189708); Brachypodium distachyon 5-pentadecatrienyl resorcinol O-methyltransferase-like (LOC100832641, ACCESSION XM_{—}003568057); Brachypodium distachyon 5-pentadecatrienyl resorcinol O-methyltransferase-like (LOC100831021, ACCESSION XM_{—}003565995); Brachypodium distachyon 5-pentadecatrienyl resorcinol O-methyltransferase-like (LOC100846894. ACCESSION XM_{—}003564128). All references and GenBank ID Nos with associated sequences mentioned above are incorporated by reference in their entireties for all purposes.

In some embodiments of this invention the plant-expressed promoter is a vascular specific or preferred promoter such as a phloem specific or preferred promoter. In some embodiments of the present invention, a phloem specific promoter is a plant-expressible promoter at least expressed in phloem cells, wherein the expression in non-phloem cells is more limited (or absent) compared to the expression in phloem cells. Examples of suitable vascular specific, vascular preferred, phloem specific, or phloem preferred promoters in accordance with this invention include, but are not limited to the promoters selected from the group consisting of: the SCSV3, SCSV4, SCSV5, and SCSV7 promoters (Schunmann et al., 2003), the rolC gene promoter of Agrobacterium rhizogenes (Kiyokawa et al., 1994; Pandolfini et al., 2003; Graham et al., 1997; Guivarc'h et al., 1996, Almon et al; 1997), the rolA gene promoter of Agrobacteriumn rhizogenes (Dehio et al., 1993), the promoter of the Agrobacteriumn tumefaciens T-DNA gene 5 (Korber et al. 1991), the rice sucrose synthase RSs1 gene promoter (Shi et al., 1994), the CoYMV or Commelina yellow mottle badnavirus promoter (Medberry et al., 1992; Zhou et al., 1998), the CFDV or coconut foliar decay virus promoter (Rohde et al., 1994; Hehn and Rhode, 1998), the RTBV or rice tungro bacilliform virus promoter (Yin and Beachy, 1995; Yin et al., 1997), the pea glutamine synthase GS3A gene promoter (Edwards et al., 1990; Brears et al., 1991), the invCD111 and invCD141 promoters of the potato invertase genes (Hedley et al., 2000), the promoter isolated from Arabidopsis shown to have phloem specific expression in tobacco by Kertbundit et al (1991), the VAHOX1 promoter region (Tomero et al., 1996), the pea cell wall invertase gene promoter (Zhang et al., 1996), the promoter of the endogenous cotton protein related to chitinase of US published patent application 20030106097, an acid invertase gene promoter from carrot (Ramloch-Lorenz et al., 1993), the promoter of the sulfate transporter gene Sultr1;3 (Yoshimoto et al., 2003), a promoter of a sucrose synthase gene (Nolte and Koch, 1993), and the promoter of a tobacco sucrose transporter gene (Kuhn et al., 1997). Also, any promoter homologous to (or having a high sequence identity to) any of the above promoters and also exhibiting phloem specific expression, as defined herein, can be used. The selection of a particular promoter can depend on the expression level and tissue distribution desired. These promoters can be combined with enhancer elements, they can be combined with minimal promoter elements, or can comprise repeated elements to ensure the expression profile desired.

More stem specific and/or stem preferred promoters have been described before in Birch & Potier, 2000; Hudspeth & Gurla (Plant Molec. Biol. 12: 579-589 (1989)); De Framond (FEBS 290: 103-106 (1991)). Yin et al., (1997, Plant Journal 12:1178-1188); Ito et al. (2000, Plant Science 155:85-100); Bhattacharya-Pakrasi, et al. (The Plant Journal, 4(1):71-79 (1993)); Keller et al. (EMBO J. 8: 1309 (1989), bean grp1.8 promoter); Feuillet et al. (Plant Mol. Biol. 27: 651 (1995), eucalyptus CAD promoter); Van der Meer et al. (The Plant Cell 4: 253 (1992)); Salehuzzaman et al. (Plant Mol. Biol. 23: 947 (1993)); Matsuda et al. (Plant Cell Physiol. 37: 215 (1996)); Grima-Pettenati et al. (Plant Science, 145: 51-65 (1999)), and also in U.S. Pat. Nos. 5,824,857, 7,232,941, 7,674,951, 7,323,622, 7,253,276, 5,495,007, 5,391,725, and U.S. Pat. Appl. Publi. Nos: 20090031452, and 20080244794. Each of the publications on tissue specific or tissue preferred promoters mentioned herein is incorporated by reference in its entirety.

In some embodiments, the stem specific and/or stem preferred promoter is associated with a DIRIGENT gene. Examples of DIRIGENT genes are described previously, e.g., in Damaj et al. (2010), including but are not limited to genes e.g., genes encoding Saccharum spp. hybrid (sugarcane hybrid) dirigent-like protein (SHDIR16, GenBank ID NO. GU062718); S. officinarum (sugarcane) dirigents, e.g., SoDIR1 (GenBank ID NO. AAR00251), SoDIR2 (GenBank ID NO. CAF25234) and SoDIR3 (GenBank ID NO. AAV50047); Zea mays (maize) dirigents: e.g., ZmDIR-A (GenBank ID NO. NM_{—}001158343), ZmDIR-B (GenBank ID NO. NM_{—}001156165), ZmDIR1 (GenBank ID NO. AAF71261), ZmDIR3 (GenBank ID NO. NM_{—}001158356) and ZmDIR9 (GenBank ID NO. NM_{—}001157043); Z. mays disease resistance-response (DRR) genes: e.g., ZmDRR1 (GenBank ID NO. NM_{—}001157553), ZmDRR2 (GenBank ID NO. NM_{—}001158590), ZmDRR3 (GenBank ID NO. NM_{—}001156097) and ZmDRR4 (GenBank ID NO. NM_{—}001158233); Sorghum bicolor (sorghum) dirigent (SbDIR1, GenBank ID NO. AAM94289); Hordeum vulgare (barley) dirigents: e.g., HvDIR1 (GenBank ID NO. AAA87042), HvDIR2 (GenBank ID NO. AAA87041) and HvDIR3 (GenBank ID NO. AAB72098); Triticum aestivum (wheat) dirigents: e.g., TaDIR1 (GenBank ID NO. AAC49284), TaDIR2 (GenBank ID NO. AAM46813), TaDIR3 (GenBank ID NO. BAA32786) and TaDIR4 (GenBank ID NO. AAR20919); Oryza sativa (rice) dirigents: e.g., OsDIR5 (GenBank ID NO. AK108922), OsDIR11 (GenBank ID NO. AK106022) and OsDIR15 (GenBank ID NO. AK108983); O. sativa DRRs: e.g., OsDRR1 (GenBank ID NO. AC115686/AAM74358), OsDRR3 (GenBank ID NO. AP003749.3/BAC16397), OsDRR4 (GenBank ID NO. AP003749.3/BAC16399), OsDRR5 (GenBank ID NO. AP005292.5/BAC45193), OsDRR6 (GenBank ID NO. CM000132.1/EEC82520), OsDRR7 (GenBank ID NO. AP005292.5/BAC45199), OsDRR9 (GenBank ID NO. CM000144/EAZ40847), Os-DRR10 (GenBank ID NO. AP003765.5/BAC19943) and OsDRR11 (GenBank ID NO. AP004342.5/BAC20739); Thuja plicata (western red cedar) dirigents: e.g., TpDIR1 (GenBank ID NO. AAF25359), TpDIR2 (GenBank ID NO. AAF25360), TpDIR3 (GenBank ID NO. AAF25361), TpDIR4 (GenBank ID NO. AAF25362), TpDIR5 (GenBank ID NO. AAF25363), TpDIR6 (GenBank ID NO. AAF25364), TpDIR7 (GenBank ID NO. AAF25365), TpDIR8 (GenBank ID NO. AAF25366) and TpDIR9 (GenBank ID NO. AAL92120); Tsuga heterophylla (western hemlock) dirigent (ThDIR1, GenBank ID NO. AAF25367); Forsythia×internedia (border Forsythia shrub) dirigents: e.g., FiDIR1 (GenBank ID NO. AF210061) and FiDIR2 (GenBank ID NO. AF210062); Pisum sativum (pea) DRR (GenBank ID NO. PsDRR206-d, PSU11716); Populus trichocarpa (western balsam poplar) DRRs: e.g., PtDRR1 (GenBank ID NO. XM_{—}002297959), PtDRR2 (GenBank ID NO. XM_{—}002297960), PtDRR3 (GenBank ID NO. XM_{—}002297961), PtDRR5 (GenBank ID NO. XM_{—}002303581) and PtDRR6 (GenBank ID NO. XM_{—}002304498); Podophyllum peltatum (mayapple) dirigent (PpDIR, GenBank ID NO. AF352736); and Agrostis stolonifera (bentgrass) dirigent (AsDIR1, GenBank ID NO. AAY41607). All references and GenBank ID Nos with associated sequences mentioned above are incorporated by reference in their entireties for all purposes.

Optionally, the nucleic acid sequence encoding chimeric sucrose isomerase is also operably linked to a plant 3′ non-translated region (3′ UTR). A plant 3′ non-translated sequence is not necessarily derived from a plant gene. For example, it can be a terminator sequence derived from viral or bacterium gene, or T-DNA. The 3′ non-translated regulatory DNA sequence can include from about 20 to 50, about 50 to 100, about 100 to 500, or about 500 to 1,000 nucleotide base pairs and may contain plant transcriptional and translational termination sequences in addition to a polyadenylation signal and any other regulatory signals capable of effecting mRNA processing or gene expression. Non-limiting examples of suitable 3′ non-translated sequences are the 3′ transcribed non-translated regions containing a polyadenylation signal from the nopaline synthase (NOS) gene of Agrobacterium tumefaciens (Bevan et al., 1983, Nucl. Acid Res., 11:369), or terminator for the T7 transcript from the octopine synthase gene of Agrobacterium tumefaciens. More suitable 3′ non-translated sequences include, 3′UTR of the potato cathepsin D inhibitor gene (GenBank Acc. No.: X74985), 3′UTR of the field bean storage protein gene VfLEIB3 (GenBank Acc. No.: Z26489), 3′UTR of pea E9 small subunit of the ribulose-1,5-bisphosphate carboxylase (ssRUBISCO) gene, 3′UTR of pea bcs, the tml terminator, the AHAS large and small subunit terminators, and OCS gene (octopene synthase) terminator. Each of the publications on plant 3′ non-translated region mentioned herein is incorporated by reference in its entirety. The plant 3′ non-translated regions and plant promoters mentioned herein can be used in vectors for both monocotyledon and dicotyledon transformations.

The expression vectors of the present invention further comprise nucleic acids encoding one or more selection markers. The selection marker can be a positive selectable marker, a negative selectable marker, or combination thereof. A “positive selectable marker gene” encodes a protein that allows growth on selective medium of cells that carry the marker gene, but not of cells that do not carry the marker gene. Selection is for cells that grow on the selective medium (showing acquisition of the marker) and is used to identify transformants. A common example is a drug-resistance marker such as NPT (neomycin phosphotransferase), whose gene product detoxifies kanamycin by phosphorylation and thus allows growth on media containing the drug. Other positive selectable marker genes for use in connection with the present invention include, but are not limited to, a 7leo gene (Potrykus et al., 1985), which codes for kanamycin resistance and can be selected for using kanamycin, G418, etc.; a bar gene, which codes for bialaphos (basta) resistance; a mutant aroA gene, which encodes an altered EPSP synthase protein (Hinchee et al., 1988), thus conferring glyphosate resistance; a nitrilase gene such as bxn from Klebsiella ozaenae, which confers resistance to bromoxynil (Stalker et al., 1988); a mutant acetolactate synthase gene (ALS), which confers resistance to imidazolinone, sulfonylurea or other ALS inhibiting chemicals (European Patent Application 154,204,1985); a methotrexate resistant DHFR gene (Thillet et al., 1988), or a dalapon dehalogenase gene that confers resistance to the herbicide dalapon; the pat gene from Streptomyces viridochromogenes, which encodes the enzyme phosphinothricin acetyl transferase (PAT) and inactivates the active ingredient in the herbicide bialaphos, phosphinothricin (PPT); or a mutated anthranilate synthase gene that confers resistance to 5-methyl tryptophan. Additional positive selectable marker genes include those genes that provide resistance to environmental factors such as excess moisture, chilling, freezing, high temperature, salt, and oxidative stress. Of course, when it is desired to introduce such a trait into a plant as a “gene of interest”, the selectable marker cannot be one that provides for resistance to an environmental factor.

Markers useful in the practice of the claimed invention include: an “antifreeze” protein such as that of the winter flounder (Cutler et al., 1989) or synthetic gene derivatives thereof; genes which provide improved chilling tolerance, such as that conferred through increased expression of glycerol-3-phosphate acetyltransferase in chloroplasts (Murata et al., 1992; Wolter et al., 1992); resistance to oxidative stress conferred by expression of superoxide dismutase (Gupta et al., 1993), and may be improved by glutathione reductase (Bowler et al., 1992); genes providing “drought resistance” and “drought tolerance”, such as genes encoding for mannitol dehydrogenase (Lee and Saier, 1982) and trehalose-6-phosphate synthase (Kaasen et al., 1992).

A “negative selectable marker gene” encodes a protein that prevents the growth of a plant or plant cell on selective medium of plants that carry the marker gene, but not of plants that do not carry the marker gene. Selection of plants that grow on the selective medium provides for the identification of plants that have eliminated or evicted the selectable marker genes. An example is CodA (Escherichia coli cytosine deaminase), whose gene product deaminates 5-fluorocytosine (which is normally non-toxic as plants do not metabolize cytosine) to the toxic 5-fluorouracil. Other negative selectable markers include the haloalkane dehalogenase (dhlA) gene of Xanthobacter autotrophicus GJ10 which encodes a dehalogenase, which hydrolyzes dihaloalkanes, such as 1,2-dichloroethane (DCE), to a halogenated alcohol and an inorganic halide (Naested et al., 1999, Plant J. 18 (5): 571-6). Each of the publications on selectable markers mentioned herein is incorporated by reference in its entirety.

Optionally, additional nucleic acid sequence can be included into the expression vectors of the present invention to facilitate the transcription, translation, and post-translational modification, so that expression and accumulation of active sucrose isomerase in a plant cell are increased. Such additional nucleic acid sequence can enhance either the expression, or the stability of the protein. In some embodiments, such nucleic acid is an intron that has positive effect on gene expression, which has been also known as intron-mediated enhancement (IME, see Mascarenhas et al., (1990). Plant Mol. Biol. 15: 913-920). IME has been observed in a wide range of eukaryotes, including vertebrates, invertebrates, fungi, and plants (see references 17-26), suggesting that it reflects a fundamental feature of gene expression. In many cases, introns have a larger influence than do promoters in determining the level and pattern of expression. Non-limiting IME in plants have been described in Rose et al. (The Plant Cell 20:543-551 (2008)); Lee et al. (Plant Physiology 145:1294-1300 (2007)); Casas-Mollano et al. (Journal of Experimental Botany Volume 57, Number 12 Pp. 3301-3311); Jeong et al. (Plant Physiology 140:196-209 (2006)); Clancy et al. (Plant Physiol, October 2002, Vol. 130, pp. 918-929); Jeon et al. (Plant Physiol, July 2000, Vol. 123, pp. 1005-1014); Rose et al. (Plant Physiol, February 2000, Vol. 122, pp. 535-542); Kim et al. (Plant Physiol. (1999) 121: 225-236), and Callis et al. (Genes Dev. 1987 1: 1183-1200). Each of the publications on IMEs mentioned herein is incorporated by reference in its entirety. Thus, in some embodiments, any one of the IME described herein can be included in the expression vectors of the present invention. For example, the first intron (SEQ ID NO: 25) of ADH1 (Alcohol Dehydrogenase 1) gene can be included upstream of the initiator methionine to increase expression (see Callis et al., Genes Dev. 1987 1: 1183-1200).

The expression vectors of the present invention can be transformed into a plant to increase the seed weight, seed number and/or seed size thereof, using the transformation methods described separately below. Thus, the present invention provides transgenic plants transformed with the expression vectors as described herein. The plant can be any plant in which an increased seed weight, seed number and/or seed size is preferred by breeders for any reasons, e.g., for economical/agricultural interests. In some embodiments, said plants are dicotyledon plants. For example, the plant is a bean plant, a soybean plant, peanuts, nuts, members of the Brassicaceae family (Camelina, oilseed rape, Canola, etc.), amaranth, cotton, peas, tomatoes, sugarbeet, sunflower. In another embodiment, said plants are monocotyledon plants. For example, the plant is corn, rice, wheat, barley, sorghum, millets, oats, ryes, triticales, buckwheats, fonio, quinoa, oil palm.

Methods of Increasing Average Seed Weight, Seed Number and/or Seed Size

The present invention provides methods of increasing seed weight, seed number and/or size. In some embodiments, the methods comprise incorporating the recombinant sucrose isomerase of the present invention as described herein into a plant. One skilled in the art would be able to select suitable methods of incorporation.

The recombinant sucrose isomerase can be incorporated into a plant by transforming the plant with an expression vector of the present invention as described elsewhere herein. The recombinant sucrose isomerase can also be incorporated into a plant by breeding methods. For example, a transgenic plant comprising the recombinant sucrose isomerase of the present invention can be crossed to a second plant to produce a progeny wherein new transgenic plants comprising the recombinant sucrose isomerase can be isolated. Methods of breeding are discussed separately below.

Any transgenic plant with increased seed weight, seed number and/or seed size generated from the present invention comprising a recombinant sucrose isomerase can be used as a donor to produce more transgenic plants through plant breeding methods well known to those skilled in the art. The goal in general is to develop new, unique and superior varieties and hybrids. In some embodiments, selection methods, e.g., molecular marker assisted selection, can be combined with breeding methods to accelerate the process.

In some embodiments, said method comprises (i) crossing any one of the plants of the present invention comprising a recombinant sucrose isomerase with increased seed weight, seed number and/or seed size as a donor to a recipient plant line to create a F1 population; (ii) evaluating seed weight, seed number and/or seed size in the offsprings derived from said F1 population; and (iii) selecting offsprings that have increased seed weight, seed number and/or seed size.

In some embodiments, complete chromosomes of the donor plant are transferred. For example, the transgenic plant with increased seed weight, seed number and/or seed size can serve as a male or female parent in a cross pollination to produce offspring plants, wherein by receiving the transgene from the donor plant, the offspring plants have increased seed weight, seed number and/or seed size.

In a method for producing plants having increased seed weight, seed number and/or seed size, protoplast fusion can also be used for the transfer of the transgene from a donor plant to a recipient plant. Protoplast fusion is an induced or spontaneous union, such as a somatic hybridization, between two or more protoplasts (cells of which the cell walls are removed by enzymatic treatment) to produce a single bi- or multi-nucleate cell. The fused cell, that may even be obtained with plant species that cannot be interbred in nature, is tissue cultured into a hybrid plant exhibiting the desirable combination of traits. More specifically, a first protoplast can be obtained from a plant having increased seed weight, seed number and/or seed size. A second protoplast can be obtained from a second plant line, optionally from another plant species or variety, preferably from the same plant species or variety, that comprises commercially desirable characteristics, such as, but not limited to disease resistance, insect resistance, valuable grain characteristics (e.g., increased seed weight, seed number and/or seed size) etc. The protoplasts are then fused using traditional protoplast fusion procedures, which are known in the art to produce the cross.

Alternatively, embryo rescue may be employed in the transfer of recombinant sucrose isomerase from a donor plant to a recipient plant. Embryo rescue can be used as a procedure to isolate embryo's from crosses wherein plants fail to produce viable seed. In this process, the fertilized ovary or immature seed of a plant is tissue cultured to create new plants (see Pierik, 1999, In vitro culture of higher plants, Springer, ISBN 079235267x, 9780792352679, which is incorporated herein by reference in its entirety).

In some embodiments, the recipient plant is an elite line having one or more certain agronomically important traits. As used herein, “agronomically important traits” include any phenotype in a plant or plant part that is useful or advantageous for human use. Examples of agronomically important traits include but are not limited to those that result in increased biomass production, production of specific biofuels, increased food production, improved food quality, increased seed oil content, etc. Additional examples of agronomically important traits includes pest resistance, vigor, development time (time to harvest), enhanced nutrient content, novel growth patterns, flavors or colors, salt, heat, drought and cold tolerance, and the like. Agronomically important traits do not include selectable marker genes (e.g., genes encoding herbicide or antibiotic resistance used only to facilitate detection or selection of transformed cells), hormone biosynthesis genes leading to the production of a plant hormone (e.g., auxins, gibberellins, cytokinins, abscisic acid and ethylene that are used only for selection), or reporter genes (e.g. luciferase, β-glucuronidase, chloramphenicol acetyl transferase (CAT, etc.). For example, the recipient plant can be a plant with increased seed weight, seed number and/or seed size which is due to a trait not related to sucrose isomerase, such as traits in the plants created by the KRP protein related techniques and REVOLUTA protein related techniques described in WO 2007/016319 and WO 2007/079353, which are incorporated herein by reference in their entireties. The recipient plant can also be a plant with preferred carbohydrate composition, e.g., composition preferred for nutritional or industrial applications, especially those plants in which the preferred composition is present in seeds.

Transgenic Plants with Increased Average Seed Weight, Seed Number and/or Seed Size

The present invention provides transgenic plants expressing a sucrose isomerase, biologically active variants, or fragments thereof, wherein the transgenic plant has increased seed weight, seed number and/or seed size compared to a control plant not expressing the sucrose isomerase, biologically active variants, or fragments thereof. In some embodiments, said plant is a dicotyledon plant. In another embodiment, said plant is a monocotyledon plant. The plant can be any plant wherein an increased seed weight, seed number and/or seed size are of interest. For example, the plant is a dicotyledon plant, e.g., bean, soybean, peanut, nuts, members of the Brassicaceae family (Camelina, oilseed rape, Canola, etc.), amaranth, cotton, peas, sunflower, or a monocotyledon plant, such as a cereal crop, e.g., corn, rice, wheat, barley, sorghum, millets, oats, ryes, triticales, buckwheats, fonio, quinoa, or oil palm.

In some embodiments, the seed weight, seed number and/or seed size of the corn plant increases at least 5%, at least 6%, 7%. 8%, 9%, 10%, 11%. 12%, 13%, 14%, 15%, 16%. 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%. 40%, 41%, 42%, 43%, 44%. 45%, 46%, 47%, 48%, 49%. 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 110%, 120%. 130%, 140%, 150%, 160%. 170%, 180%, 190%, 200%. 210%, 220%, 230%, 240%, 250%, 260%, 270%, 280%, 290%, 300%, 310%, 320%, 330%, 340%, 350%, 360%, 370%, 380%, 390%, 400%, or more compared to a control plant. The control plant is a plant which does not express a sucrose isomerase, biologically active variants, or fragments thereof.

In other embodiments, new plants can be derived from a cross wherein at least one parent is the transgenic plants of the present invention with increased seed weight, seed number and/or seed size as described herein using breeding methods described elsewhere herein. Additional breeding methods have been known to one of ordinary skill in the art, e.g., methods discussed in Chahal and Gosal (Principles and procedures of plant breeding: biotechnological and conventional approaches, CRC Press, 2002, ISBN 084931321X, 9780849313219), Taji et al. (In vitro plant breeding, Routledge, 2002, ISBN 156022908X, 9781560229087), Richards (Plant breeding systems, Taylor & Francis U S, 1997, ISBN 0412574500, 9780412574504), Hayes (Methods of Plant Breeding, Publisher: READ BOOKS, 2007. ISBN1406737062, 9781406737066), each of which is incorporated by reference in its entirety.

The present invention also provides a seed, a fruit, a plant population, a plant part, a plant cell and/or a plant tissue culture derived from the transgenic plants as described herein.

Modern plant tissue culture is performed under aseptic conditions under filtered air. Living plant materials from the environment are naturally contaminated on their surfaces (and sometimes interiors) with microorganisms, so surface sterilization of starting materials (explants) in chemical solutions (usually alcohol or bleach) is required. Explants are then usually placed on the surface of a solid culture medium, but are sometimes placed directly into a liquid medium, particularly when cell suspension cultures are desired. Solid and liquid media are generally composed of inorganic salts plus a few organic nutrients, vitamins and plant hormones. Solid media are prepared from liquid media with the addition of a gelling agent, usually purified agar.

The composition of the medium, particularly the plant hormones and the nitrogen source (nitrate versus ammonium salts or amino acids) have profound effects on the morphology of the tissues that grow from the initial explant. For example, an excess of auxin will often result in a proliferation of roots, while an excess of cytokinin may yield shoots. A balance of both auxin and cytokinin will often produce an unorganized growth of cells, or callus, but the morphology of the outgrowth will depend on the plant species as well as the medium composition. As cultures grow, pieces are typically sliced off and transferred to new media (subcultured) to allow for growth or to alter the morphology of the culture. The skill and experience of the tissue culturist are important in judging which pieces to culture and which to discard. As shoots emerge from a culture, they may be sliced off and rooted with auxin to produce plantlets which, when mature, can be transferred to potting soil for further growth in the greenhouse as normal plants.

The transgenic plants of the present invention can be used for many purposes. In some embodiments, the transgenic plant is used as a donor plant of genetic material which can be transferred to a recipient plant to produce a plant which has the transferred genetic material and has also increased seed weight, seed number and/or seed size. Any suitable method known in the art can be applied to transfer genetic material from a donor plant to a recipient plant. In most cases, such genetic material is genomic material.

In some embodiments, the whole genome of the transgenic plants of the present invention is transferred into a recipient plant. This can be done by crossing the transgenic plants to a recipient plant to create a F1 plant. The F1 plant can be further selfed and selected for one, two, three, four, or more generations to give plants with increased seed weight, seed number and/or seed size.

In another embodiment, at least the parts containing the transgene of the donor plant's genome are transferred. This can be done by crossing the transgenic plants to a recipient plant to create a F1 plant, followed with one or more backcrosses to one of the parent plants to plants with the desired genetic background. The progeny resulting from the backcrosses can be further selfed and selected to give plants with increased seed weight, seed number and/or seed size. In some embodiments, the recipient plant is an elite line having one or more certain agronomically important traits.

The transgenic plants of the present invention may have altered metabolic profiles compared to a control plant. For example, the transgenic plants of the present invention may contain certain levels of isomaltulose and trehalulose, which are not normally present in plants. Thus, in some embodiments, the transgenic plants of the present invention can be used to produce isomaltulose and/or trehalulose, which have many industrial applications. In some embodiments, the transgenic plants of the present invention have increased or decreased sucrose compared to a control plant. In some embodiments, the transgenic plants of the present invention have increased or decreased disaccharide contents compared to a control plant.

A plant with altered metabolic profiles can be selected by methods well known to one skilled in the art. For example, metabolic profiles can be screened using quantitative chemical analysis methods, e.g., using gas chromatography (GC) analysis, or high performance liquid chromatography analysis (or high pressure liquid chromatography, HPLC).

A gas chromatograph is a chemical analysis instrument for separating chemicals in a complex sample. A gas chromatograph uses a flow-through narrow tube known as the column, through which different chemical constituents of a sample pass in a gas stream (carrier gas, mobile phase) at different rates depending on their various chemical and physical properties and their interaction with a specific column filling, called the stationary phase. As the chemicals exit the end of the column, they are detected and identified electronically. The function of the stationary phase in the column is to separate different components, causing each one to exit the column at a different time (retention time). Other parameters that can be used to alter the order or time of retention are the carrier gas flow rate, and the temperature. In a GC analysis, a known volume of gaseous or liquid analyte is injected into the “entrance” (head) of the column, usually using a microsyringe (or, solid phase microextraction fibers, or a gas source switching system). As the carrier gas sweeps the analyte molecules through the column, this motion is inhibited by the adsorption of the analyte molecules either by adherence onto the column walls or onto packing materials in the column. The rate at which the molecules progress along the column depends on the strength of adsorption, which in turn depends on the type of molecule and on the stationary phase materials. Since each type of molecule has a different rate of progression, the various components of the analyte mixture are separated as they progress along the column and reach the end of the column at different times (retention time). A detector is used to monitor the outlet stream from the column; thus, the time at which each component reaches the outlet and the amount of that component can be determined. Generally, substances are identified (qualitatively) by the order in which they emerge (elute) from the column and by the retention time of the analyte in the column. Detailed methods of performing GC analysis can be found in Pavia et al., (2006, Introduction to Organic Laboratory Techniques (4th Ed.). ISBN 978-0-495-28069-9) and Harris (1999, Quantitative chemical analysis (Fifth ed.), Chapter 24, W. H. Freeman and Company, pp. 675-712, ISBN 0-7167-2881-8), which are incorporated by reference in their entireties.

HPLC is a form of column chromatography used frequently in biochemistry and analytical chemistry to separate, identify, and quantify compounds based on their idiosyncratic polarities and interactions with the column's stationary phase. HPLC utilizes different types of stationary phase (typically, hydrophobic saturated carbon chains), a pump that moves the mobile phase(s) and analyte through the column, and a detector that provides a characteristic retention time for the analyte. The detector may also provide other characteristic information (i.e. UV/Vis spectroscopic data for analyte if so equipped). Analyte retention time varies depending on the strength of its interactions with the stationary phase, the ratio/composition of solvent(s) used, and the flow rate of the mobile phase. Non-limiting exemplary HPLC types include, partition chromatography, normal phase chromatography, displacement chromatography, reversed phase chromatography (RPC), size exclusion chromatography, ion exchange chromatography, bioaffinity chromatography, and aqueous normal phase chromatography. HPLC chromatographs are commercially produced at least by several companies, e.g., Agilent Technologies, Shimadzu Scientific Instruments, Beckman Coulter, Cecil Instruments, Dionex Corp, Hitachi, PerkinElmer, Inc., Thermo Fisher Scientific, Varian, Inc., and Waters Corporation. More detailed description of using HPLC can be found in Swadesh (HPLC: practical and industrial applications, Publisher: CRC Press, 2001, ISBN 0849300037, 9780849300035), MacDonald et al. (HPLC: instrumentation & applications, Publisher: International Scientific Communications, 1986), Katz (Handbook of HPLC, Publisher: CRC Press, 2002, ISBN 0824794443, 9780824794446), and Nollet (Food analysis by HPLC, Publisher: CRC Press, 2000, ISBN 082478460X, 9780824784607), each of which is incorporated by reference in its entirety.

### Plant Transformation

The polynucleotides of the present invention can be transformed into a plant. The most common method for the introduction of new genetic material into a plant genome involves the use of living cells of the bacterial pathogen Agrobacterium tumefaciens to literally inject a piece of DNA, called transfer or T-DNA, into individual plant cells (usually following wounding of the tissue) where it is targeted to the plant nucleus for chromosomal integration. There are numerous patents governing Agrobacterium mediated transformation and particular DNA delivery plasmids designed specifically for use with Agrobacterium—for example, U.S. Pat. No. 4,536,475, EP0265556, EP0270822, WO8504899, WO8603516, U.S. Pat. No. 5,591,616, EP0604662, EP0672752, WO8603776, WO9209696, WO9419930, WO9967357, U.S. Pat. No. 4,399,216, WO8303259, U.S. Pat. No. 5,731,179, EP068730, WO9516031, U.S. Pat. No. 5,693,512, U.S. Pat. No. 6,051,757 and EP904362A 1. Agrobacterium-mediated plant transformation involves as a first step the placement of DNA fragments cloned on plasmids into living Agrobacterium cells, which are then subsequently used for transformation into individual plant cells. Agrobacterium-mediated plant transformation is thus an indirect plant transformation method. Methods of Agrobacterium-mediated plant transformation that involve using vectors with no T-DNA are also well known to those skilled in the art and can have applicability in the present invention. See, for example, U.S. Pat. No. 7,250,554, which utilizes P-DNA instead of T-DNA in the transformation vector.

Direct plant transformation methods using DNA have also been reported. The first of these to be reported historically is electroporation, which utilizes an electrical current applied to a solution containing plant cells (M. E. Fromm et al., Nature, 319, 791 (1986); H. Jones et al., Plant Mol. Biol., 13, 501 (1989) and H. Yang et al., Plant Cell Reports, 7, 421 (1988). Another direct method, called “biolistic bombardment”, uses ultrafine particles, usually tungsten or gold, that are coated with DNA and then sprayed onto the surface of a plant tissue with sufficient force to cause the particles to penetrate plant cells, including the thick cell wall, membrane and nuclear envelope, but without killing at least some of them (U.S. Pat. No. 5,204,253, U.S. Pat. No. 5,015,580). A third direct method uses fibrous forms of metal or ceramic consisting of sharp, porous or hollow needle-like projections that literally impale the cells, and also the nuclear envelope of cells. Both silicon carbide and aluminum borate whiskers have been used for plant transformation (Mizuno et al., 2004; Petolino et al., 2000; U.S. Pat. No. 5,302,523 US Application 20040197909) and also for bacterial and animal transformation (Kaepler et al., 1992; Raloff, 1990; Wang, 1995). There are other methods reported, and undoubtedly, additional methods will be developed. However, the efficiencies of each of these indirect or direct methods in introducing foreign DNA into plant cells are invariably extremely low, making it necessary to use some method for selection of only those cells that have been transformed, and further, allowing growth and regeneration into plants of only those cells that have been transformed.

For efficient plant transformation, a selection method must be employed such that whole plants are regenerated from a single transformed cell and every cell of the transformed plant carries the DNA of interest. These methods can employ positive selection, whereby a foreign gene is supplied to a plant cell that allows it to utilize a substrate present in the medium that it otherwise could not use, such as mannose or xylose (for example, refer U.S. Pat. No. 5,767,378; U.S. Pat. No. 5,994,629). More typically, however, negative selection is used because it is more efficient, utilizing selective agents such as herbicides or antibiotics that either kill or inhibit the growth of nontransformed plant cells and reducing the possibility of chimeras. Resistance genes that are effective against negative selective agents are provided on the introduced foreign DNA used for the plant transformation. For example, one of the most popular selective agents used is the antibiotic kanamycin, together with the resistance gene neomycin phosphotransferase (nptII), which confers resistance to kanamycin and related antibiotics (see, for example, Messing & Vierra, Gene 19: 259-268 (1982); Bevan et al., Nature 304:184-187 (1983)). However, many different antibiotics and antibiotic resistance genes can be used for transformation purposes (refer U.S. Pat. No. 5,034,322, U.S. Pat. No. 6,174,724 and U.S. Pat. No. 6,255,560). In addition, several herbicides and herbicide resistance genes have been used for transformation purposes, including the bar gene, which confers resistance to the herbicide phosphinothricin (White et al., Nucl Acids Res 18: 1062 (1990), Spencer et al., Theor Appl Genet. 79: 625-631 (1990), U.S. Pat. No. 4,795,855, U.S. Pat. No. 5,378,824 and U.S. Pat. No. 6,107,549). In addition, the dhfr gene, which confers resistance to the anticancer agent methotrexate, has been used for selection (Bourouis et al., EMBO J. 2(7): 1099-1104 (1983).

The expression control elements used to regulate the expression of a given protein can either be the expression control element that is normally found associated with the coding sequence (homologous expression element) or can be a heterologous expression control element. A variety of homologous and heterologous expression control elements are known in the art and can readily be used to make expression units for use in the present invention. Transcription initiation regions, for example, can include any of the various opine initiation regions, such as octopine, mannopine, nopaline and the like that are found in the Ti plasmids of Agrobacterium tumefaciens. Alternatively, plant viral promoters can also be used, such as the cauliflower mosaic virus 19S and 35S promoters (CaMV 19S and CaMV 35S promoters, respectively) to control gene expression in a plant (U.S. Pat. Nos. 5,352,605; 5,530,196 and 5,858,742 for example). Enhancer sequences derived from the CaMV can also be utilized (U.S. Pat. Nos. 5,164,316; 5,196.525; 5,322,938; 5,530,196; 5,352,605; 5,359,142; and 5,858,742 for example). Lastly, plant promoters such as prolifera promoter, fruit specific promoters, Ap3 promoter, heat shock promoters, seed specific promoters, etc. can also be used.

Either a gamete specific promoter, a constitutive promoter (such as the CaMV or Nos promoter), an organ specific promoter (e.g., stem specific promoter), or an inducible promoter is typically ligated to the protein or antisense encoding region using standard techniques known in the art. The expression unit may be further optimized by employing supplemental elements such as transcription terminators and/or enhancer elements. The expression cassette can comprise, for example, a seed specific promoter (e.g. the phaseolin promoter (U.S. Pat. No. 5,504,200). The term “seed specific promoter”, means that a gene expressed under the control of the promoter is predominantly expressed in plant seeds with no or no substantial expression, typically less than 10% of the overall expression level, in other plant tissues. Seed specific promoters have been well known in the art, for example, U.S. Pat. Nos. 5,623,067, 5,717,129, 6,403,371, 6,566,584, 6,642,437, 6,777,591, 7,081,565, 7,157,629, 7,192,774, 7,405,345, 7,554,006, 7,589,252, 7,595,384, 7,619,135, 7,642,346, and US Application Publication Nos. 20030005485, 20030172403, 20040088754, 20040255350, 20050125861, 20050229273, 20060191044, 20070022502, 20070118933, 20070199098, 20080313771, and 20090100551.

Thus, for expression in plants, the expression units will typically contain, in addition to the protein sequence, a plant promoter region, a transcription initiation site and a transcription termination sequence. Unique restriction enzyme sites at the 5′ and 3′ ends of the expression unit are typically included to allow for easy insertion into a preexisting vector.

In the construction of heterologous promoter/structural gene or antisense combinations, the promoter is preferably positioned about the same distance from the heterologous transcription start site as it is from the transcription start site in its natural setting. As is known in the art, however, some variation in this distance can be accommodated without loss of promoter function.

In addition to a promoter sequence, the expression cassette can also contain a transcription termination region downstream of the structural gene to provide for efficient termination. The termination region may be obtained from the same gene as the promoter sequence or may be obtained from different genes. If the mRNA encoded by the structural gene is to be efficiently processed, DNA sequences which direct polyadenylation of the RNA are also commonly added to the vector construct. Polyadenylation sequences include, but are not limited to the Agrobacterium octopine synthase signal (Gielen et al., EMBO J 3:835-846 (1984)) or the nopaline synthase signal (Depicker et al., Mol. and Appl. Genet. 1:561-573 (1982)). The resulting expression unit is ligated into or otherwise constructed to be included in a vector that is appropriate for higher plant transformation. One or more expression units may be included in the same vector. The vector will typically contain a selectable marker gene expression unit by which transformed plant cells can be identified in culture. Usually, the marker gene will encode resistance to an antibiotic, such as G418, hygromycin, bleomycin, kanamycin, or gentamicin or to an herbicide, such as glyphosate (Round-Up) or glufosinate (BASTA) or atrazine. Replication sequences, of bacterial or viral origin, are generally also included to allow the vector to be cloned in a bacterial or phage host, preferably a broad host range prokaryotic origin of replication is included. A selectable marker for bacteria may also be included to allow selection of bacterial cells bearing the desired construct. Suitable prokaryotic selectable markers include resistance to antibiotics such as ampicillin, kanamycin or tetracycline. Other DNA sequences encoding additional functions may also be present in the vector, as is known in the art. For instance, in the case of Agrobacterium transformations, T-DNA sequences will also be included for subsequent transfer to plant chromosomes.

To introduce a desired gene or set of genes by conventional methods requires a sexual cross between two lines, and then repeated back-crossing between hybrid offspring and one of the parents until a plant with the desired characteristics is obtained. This process, however, is restricted to plants that can sexually hybridize, and genes in addition to the desired gene will be transferred.

Recombinant DNA techniques allow plant researchers to circumvent these limitations by enabling plant geneticists to identify and clone specific genes for desirable traits, such as resistance to an insect pest, and to introduce these genes into already useful varieties of plants. Once the foreign genes have been introduced into a plant, that plant can then be used in conventional plant breeding schemes (e.g., pedigree breeding, single-seed-descent breeding schemes, reciprocal recurrent selection) to produce progeny which also contain the gene of interest.

Genes can be introduced in a site directed fashion using homologous recombination. Homologous recombination permits site specific modifications in endogenous genes and thus inherited or acquired mutations may be corrected, and/or novel alterations may be engineered into the genome. Homologous recombination and site-directed integration in plants are discussed in, for example, U.S. Pat. Nos. 5,451,513; 5,501,967 and 5,527,695.

Methods of producing transgenic plants are well known to those of ordinary skill in the art. Transgenic plants can now be produced by a variety of different transformation methods including, but not limited to, electroporation; microinjection; microprojectile bombardment, also known as particle acceleration or biolistic bombardment; viral-mediated transformation; and Agrobacterium-mediated transformation. See, for example, U.S. Pat. Nos. 5,405,765; 5,472,869; 5,538,877; 5,538,880; 5,550,318; 5,641,664; 5,736,369 and 5,736,369; International Patent Application Publication Nos. WO2002/038779 and WO/2009/117555; Lu et al., (Plant Cell Reports, 2008, 27:273-278); Watson et al., Recombinant DNA, Scientific American Books (1992); Hinchee et al., Bio/Tech. 6:915-922 (1988); McCabe et al., Bio/Tech. 6:923-926 (1988); Toriyama et al., Bio/Tech. 6:1072-1074 (1988); Fromm et al., Bio/Tech. 8:833-839 (1990); Mullins et al., Bio/Tech. 8:833-839 (1990); Hiei et al., Plant Molecular Biology 35:205-218 (1997); Ishida et al., Nature Biotechnology 14:745-750 (1996); Zhang et al., Molecular Biotechnology 8:223-231 (1997); Ku et al., Nature Biotechnology 17:76-80 (1999); and, Raineri et al., Bio/Tech. 8:33-38 (1990)), each of which is expressly incorporated herein by reference in their entirety.

Agrobacterium tumefaciens is a naturally occurring bacterium that is capable of inserting its DNA (genetic information) into plants, resulting in a type of injury to the plant known as crown gall. Most species of plants can now be transformed using this method, including cucurbitaceous species.

Microprojectile bombardment is also known as particle acceleration, biolistic bombardment, and the gene gun (Biolistic® Gene Gun). The gene gun is used to shoot pellets that are coated with genes (e.g., for desired traits) into plant seeds or plant tissues in order to get the plant cells to then express the new genes. The gene gun uses an actual explosive (.22 caliber blank) to propel the material. Compressed air or steam may also be used as the propellant. The Biolistic® Gene Gun was invented in 1983-1984 at Cornell University by John Sanford, Edward Wolf, and Nelson Allen. It and its registered trademark are now owned by E. I. du Pont de Nemours and Company. Most species of plants have been transformed using this method.

A transgenic plant formed using Agrobacterium transformation methods typically contains a single gene on one chromosome, although multiple copies are possible. Such transgenic plants can be referred to as being hemizygous for the added gene. A more accurate name for such a plant is an independent segregant, because each transformed plant represents a unique T-DNA integration event (U.S. Pat. No. 6,156,953). A transgene locus is generally characterized by the presence and/or absence of the transgene. A heterozygous genotype in which one allele corresponds to the absence of the transgene is also designated hemizygous (U.S. Pat. No. 6,008,437).

General transformation methods, and specific methods for transforming certain plant species (e.g., maize, rice, wheat, barley, soybean) are described in U.S. Pat. Nos. 4,940,838, 5,464,763, 5,149,645, 5,501,967, 6,265,638, 4,693,976, 5,635,381, 5,731,179, 5,693,512, 6,162,965, 5,693,512, 5,981,840, 6,420,630, 6,919,494, 6,329,571, 6,215,051, 6,369,298, 5,169,770, 5,376,543, 5,416,011, 5,569,834, 5,824,877, 5,959,179, 5,563,055, and 5,968,830, each of which is incorporated by reference in its entirety.

### Breeding Methods

Classic breeding methods can be included in the present invention to introduce one or more recombinant sucrose isomerase of the present invention into other plant varieties, or other close-related species that are compatible to be crossed with the transgenic plant of the present invention.

Open-Pollinated Populations.

The improvement of open-pollinated populations of such crops as rye, many maizes and sugar beets, herbage grasses, legumes such as alfalfa and clover, and tropical tree crops such as cacao, coconuts, oil palm and some rubber, depends essentially upon changing gene-frequencies towards fixation of favorable alleles while maintaining a high (but far from maximal) degree of heterozygosity. Uniformity in such populations is impossible and trueness-to-type in an open-pollinated variety is a statistical feature of the population as a whole, not a characteristic of individual plants. Thus, the heterogeneity of open-pollinated populations contrasts with the homogeneity (or virtually so) of inbred lines, clones and hybrids.

Population improvement methods fall naturally into two groups, those based on purely phenotypic selection, normally called mass selection, and those based on selection with progeny testing. Interpopulation improvement utilizes the concept of open breeding populations; allowing genes to flow from one population to another. Plants in one population (cultivar, strain, ecotype, or any germplasm source) are crossed either naturally (e.g., by wind) or by hand or by bees (commonly Apis mellifera L. or Megachile rotundata F.) with plants from other populations. Selection is applied to improve one (or sometimes both) population(s) by isolating plants with desirable traits from both sources.

There are basically two primary methods of open-pollinated population improvement. First, there is the situation in which a population is changed en masse by a chosen selection procedure. The outcome is an improved population that is indefinitely propagable by random-mating within itself in isolation. Second, the synthetic variety attains the same end result as population improvement but is not itself propagable as such; it has to be reconstructed from parental lines or clones. These plant breeding procedures for improving open-pollinated populations are well known to those skilled in the art and comprehensive reviews of breeding procedures routinely used for improving cross-pollinated plants are provided in numerous texts and articles, including: Allard, Principles of Plant Breeding, John Wiley & Sons, Inc. (1960); Simmonds, Principles of Crop Improvement, Longman Group Limited (1979); Hallauer and Miranda, Quantitative Genetics in Maize Breeding, Iowa State University Press (1981); and, Jensen, Plant Breeding Methodology, John Wiley & Sons, Inc. (1988).

Mass Selection.

In mass selection, desirable individual plants are chosen, harvested, and the seed composited without progeny testing to produce the following generation. Since selection is based on the maternal parent only, and there is no control over pollination, mass selection amounts to a form of random mating with selection. As stated herein, the purpose of mass selection is to increase the proportion of superior genotypes in the population.

Synthetics.

A synthetic variety is produced by crossing inter se a number of genotypes selected for good combining ability in all possible hybrid combinations, with subsequent maintenance of the variety by open pollination. Whether parents are (more or less inbred) seed-propagated lines, as in some sugar beet and beans (Vicia) or clones, as in herbage grasses, clovers and alfalfa, makes no difference in principle. Parents are selected on general combining ability, sometimes by test crosses or topcrosses, more generally by polycrosses. Parental seed lines may be deliberately inbred (e.g. by selfing or sib crossing). However, even if the parents are not deliberately inbred, selection within lines during line maintenance will ensure that some inbreeding occurs. Clonal parents will, of course, remain unchanged and highly heterozygous.

Whether a synthetic can go straight from the parental seed production plot to the farmer or must first undergo one or two cycles of multiplication depends on seed production and the scale of demand for seed. In practice, grasses and clovers are generally multiplied once or twice and are thus considerably removed from the original synthetic.

While mass selection is sometimes used, progeny testing is generally preferred for polycrosses, because of their operational simplicity and obvious relevance to the objective, namely exploitation of general combining ability in a synthetic.

The number of parental lines or clones that enter a synthetic vary widely. In practice, numbers of parental lines range from 10 to several hundred, with 100-200 being the average. Broad based synthetics formed from 100 or more clones would be expected to be more stable during seed multiplication than narrow based synthetics.

Pedigreed Varieties.

A pedigreed variety is a superior genotype developed from selection of individual plants out of a segregating population followed by propagation and seed increase of self pollinated offspring and careful testing of the genotype over several generations. This is an open pollinated method that works well with naturally self pollinating species. This method can be used in combination with mass selection in variety development. Variations in pedigree and mass selection in combination are the most common methods for generating varieties in self pollinated crops.

Hybrids.

A hybrid is an individual plant resulting from a cross between parents of differing genotypes. Commercial hybrids are now used extensively in many crops, including corn (maize), sorghum, sugarbeet, sunflower and broccoli. Hybrids can be formed in a number of different ways, including by crossing two parents directly (single cross hybrids), by crossing a single cross hybrid with another parent (three-way or triple cross hybrids), or by crossing two different hybrids (four-way or double cross hybrids).

Strictly speaking, most individuals in an out breeding (i.e., open-pollinated) population are hybrids, but the term is usually reserved for cases in which the parents are individuals whose genomes are sufficiently distinct for them to be recognized as different species or subspecies. Hybrids may be fertile or sterile depending on qualitative and/or quantitative differences in the genomes of the two parents. Heterosis, or hybrid vigor, is usually associated with increased heterozygosity that results in increased vigor of growth, survival, and fertility of hybrids as compared with the parental lines that were used to form the hybrid. Maximum heterosis is usually achieved by crossing two genetically different, highly inbred lines.

The production of hybrids is a well-developed industry, involving the isolated production of both the parental lines and the hybrids which result from crossing those lines. For a detailed discussion of the hybrid production process, see, e.g., Wright, Commercial Hybrid Seed Production 8:161-176, In Hybridization of Crop Plants.

This invention is further illustrated by the following examples which should not be construed as limiting. The contents of all references, patents and published patent applications cited throughout this application, as well as the Figures and the Sequence Listing, are incorporated herein by reference.

### EXAMPLES

### Example 1

### Recombinant Expression Vectors

Several recombinant expression vectors were constructed to express sucrose isomerase (SI) in plants. Each recombinant expression vector comprises:

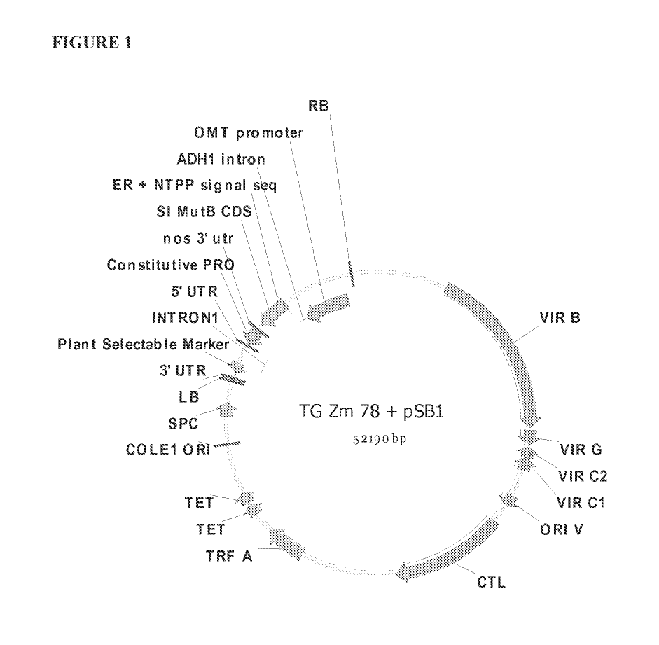

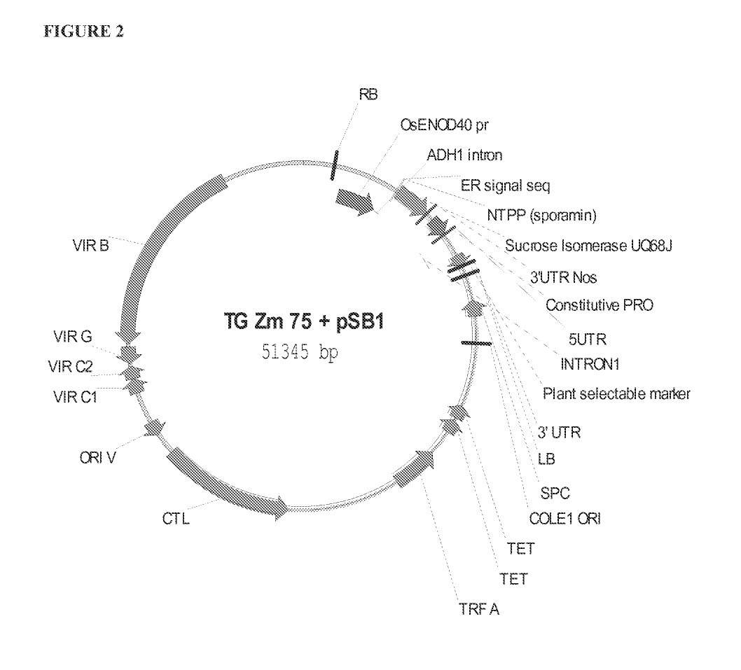

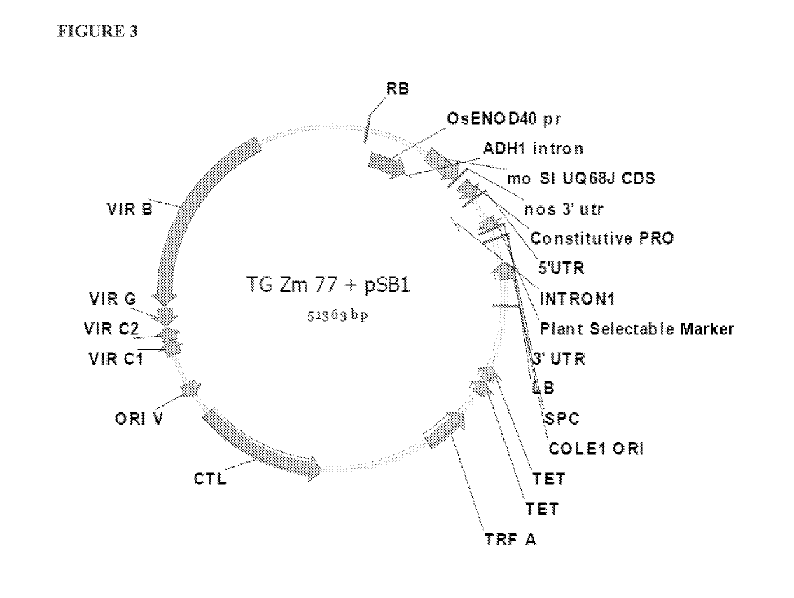

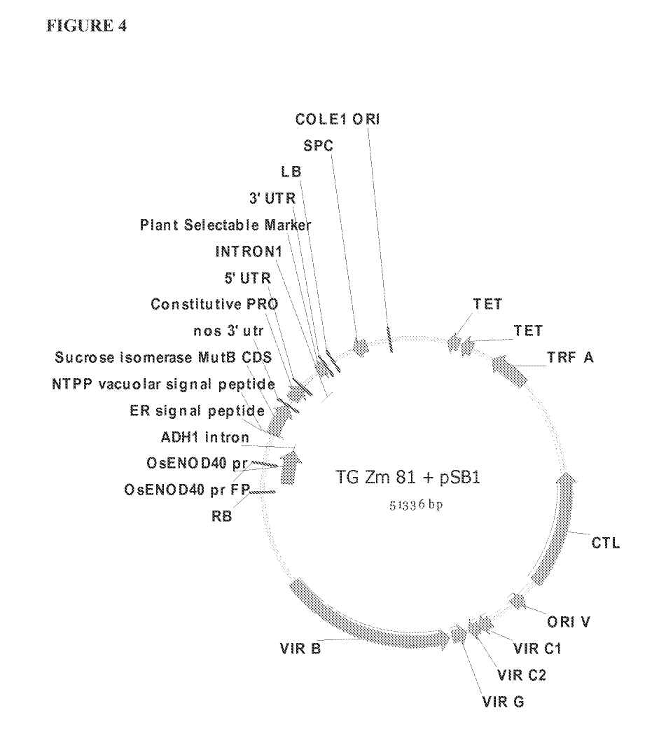

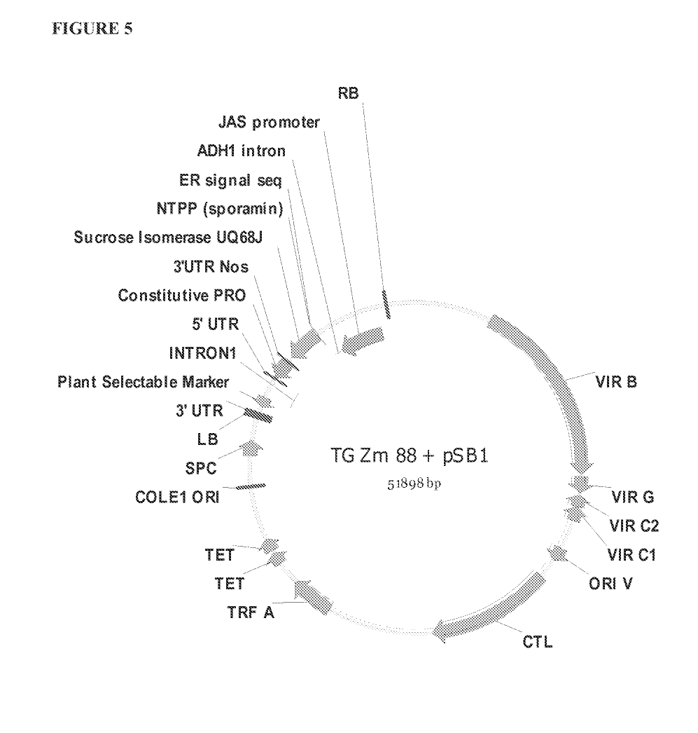

(i) a nucleic acid sequence encoding a sucrose isomerase, or biologically active variants, fragments thereof;
(ii) a nucleic acid sequence encoding a signal peptide, which is in-frame with the nucleic acid sequence encoding the sucrose isomerase, wherein the signal peptide comprises an ER signal peptide and a vacuolar targeting signal peptide;
(iii) a nucleic acid sequence of a stalk specific promoter; and
(iv) a plant selection marker gene.
Structure of the recombinant vectors for expressing sucrose isomerase in maize are shown in Table 2 below, and in FIGS. 1-5.

TABLE 2
Sucrose Isomerase Recombinant Vectors for Maize Transformation

| | Pro- | In- | Gene-nos | |
|---|---|---|---|---|
| TG_Zm # | moter | tron | 3′UTR | Source of SI Gene |
| TG_Zm #75 | ENOD40 | ADH1 | SI Pd | Pantoea dispersa |
| | | | UQ68J | UQ68J |
| TG_Zm #77 | ENOD40 | ADH1 | SI Pd | Pantoea dispersa |
| | | | moUQ68J | UQ68J |
| TG_Zm #78 | OMT | ADH1 | SI Pm | Pseudomonas |
| | | | MutB | mesoacidophila MX-45 |
| TG_ZM #81 | ENOD40 | ADH1 | SI Pm | Pseudomonas |
| | | | MutB | mesoacidophila MX-45 |
| TG_Zm #88 | JAS | ADH1 | SI Pd | Pantoea dispersa |
| | | | UQ68J | UQ68J |

The promoters used for expression in maize plants for these examples are as follows:

a) the rice stem specific promoter, ENOD40 (SEQ ID NO: 22), which in rice expresses in the xylem parenchyma cells surrounding the protoxylem of large lateral vascular bundles,
b) the o-methyl transferase promoter which is also active within parenchyma cells of the stem, and
c) the jasmonate-inducible protein promoter, which is stem preferred.

Between the promoter and the gene encoding sucrose isomerase, ADH1 (Alcohol Dehydrogenase 1) intron (SEQ ID NO: 25) was included upstream of the initiator methionine to increase expression.

Three different sucrose isomerases were used to produce transgenic plants for these examples:

a) A sucrose isomerase cloned from Pseudomonas mesoacidophila MX-45, called MutB (SEQ ID NO: 2, encoded by the nucleic acid of SEQ ID NO: 1),
b) A sucrose isomerase cloned from Pantoea dispersa, called UQ68J (SEQ ID NO: 8, encoded by the nucleic acid of SEQ ID NO: 7), and
c) A modified version of UQ68J called moUQ68J (SEQ ID NO: 13).

All three genes were codon optimized for Z. mays, and the nucleic acid sequences encoding the N-termini of these genes were deleted to produce SEQ ID NOs: 3, 9 and 14 (corresponding to MutB, UQ68J, and modified UQ68J, respectively), which were ligated in-frame to the nucleic acid sequence SEQ ID NO: 18 encoding sweet potato sporamin ER sorting signal (SEQ ID NO: 19) and nucleic acid sequence SEQ ID NO: 20 encoding sweet potato sporamin vacuolar sorting signal (NTPP, SEQ ID NO: 21), so that translation products of these three sucrose isomerase genes were targeted to the vacuoles of cells. Polypeptide sequences for codon optimized chimeric sucrose isomerases MutB, UQ68J, and modified UQ68J with ER and NTPP signal peptides at the N-termini are SEQ ID NOs: 6, 12, and 17, encoded by SEQ ID NOs: 5, 11, and 16, respectively. The maps of final recombinant vectors for transformation in maize are shown in FIGS. 1 to 5.

### Example 2

### Plant Transformation

### Maize Transformation

The constructs in superbinary Agrobacterium were maintained on minimal medium containing the antibiotics spectinomycin, rifampicin and tetracycline. Agrobacterium was streaked on LB medium with antibiotics and grown for 1-2 days.

Greenhouse-grown plants of Hi-II genotype were used as the donor material and ears were harvested 9-12 days after pollination. These were surface-sterilized with bleach solution and rinsed with sterile Milli-Q water. Immature zygotic embryos were aseptically excised from the F2 kernels of Hi-II genotype. The Agrobacteriumn from LB bacterial medium was collected and suspended in liquid infection medium and acetosyringone added to a final concentration of 100 μM. Zygotic embryos were immersed in the Agrobacterium suspension to start the bacterial infection process. Subsequently, the embryos were cultured with the scutellum side up onto the surface of co-cultivation medium and incubated in the dark for 4 days. Embryos were transferred to resting medium for 3 days followed by culturing these on selection medium containing an enzyme inhibitor used as a selection marker. Explants were sub-cultured to fresh medium every 2 weeks and maintained in the dark at 28° C. Callus resistant to the enzyme inhibitor was selected and cultured on regeneration media to initiate shoot regeneration. In most cases, multiple shoots from subcultured callus of a single source-embryo were carried through the regeneration process to produce replicate plants, or “clones”, of a single “event”. Although it is recognized that multiple clones derived from a single Agrobacterium-infected embryo do not always represent identical transgenic events of equal patterns for T-DNA integration into the maize genome, commonly this is the case.

The regenerated plants were transferred to 25×150 mm test tubes containing growth and rooting medium. Callus and leaves of regenerated plants were confirmed to be transformed. Plantlets with healthy roots were transferred into 4 inch pots containing Metro-mix 360 and maintained in the greenhouse. At 4-5 leaf stage, plants were transferred to 3 gallon pots and grown to maturity. The plants were self-pollinated and T1 seed collected ˜35 days post-pollination.

Arabidopsis thaliana Transformation

Transformation in Arabidopsis thaliana using Agrobacterium can follow the simplified protocol below. The protocol (Clough and Bent, 1998; modified from Bechtold et al. 1993) is extremely simple. The MS salts, hormone, etc. make no difference, the OD of bacteria doesn't make much of a difference, the vacuum doesn't even make much of a difference as long as one has a decent amount of surfactant present. Plant health is still a major factor. With this method one skilled in the art is able to achieve transformation rates above 1% (one transformant for every 100 seeds harvested from Agrobacterium-treated plants). The protocol is:
1. Grow healthy Arabidopsis plants until they are flowering. Grow under long days in pots in soil covered with bridal veil, window screen or cheesecloth.
2. (optional) Clip first bolts to encourage proliferation of many secondary bolts. Plants will be ready roughly 4-6 days after clipping. Clipping can be repeated to delay plants. Optimal plants have many immature flower clusters and not many fertilized siliques, although a range of plant stages can be successfully transformed.
3. Prepare Agrobacterium tumefaciens strain carrying gene of interest on a binary vector. Grow a large liquid culture at 28° C. in LB with antibiotics to select for the binary plasmid, or grow in other media. Mid-log cells or a recently stationary culture can be used.
4. Spin down Agrobacterium, resuspend to OD₆₀₀=0.8 (can be higher or lower) in 5% Sucrose solution (if made fresh, no need to autoclave). 100-200 ml for each two or three small pots are needed to be dipped, or 400-500 ml for each two or three 3.5″ (9 cm) pots.
5. Before dipping, add Silwet L-77 to a concentration of 0.05% (500 ul/L) and mix well. If there are problems with L-77 toxicity, use 0.02% or as low as 0.005%.
6. Dip above-ground parts of plant in Agrobacterium solution for 2 to 3 seconds, with gentle agitation. A film of liquid coating plant can be seen. Some investigators dip inflorescences only, while others also dip rosettes to hit the shorter axillary inflorescences.
7. Place dipped plants under a dome or cover for 16 to 24 hours to maintain high humidity (plants can be laid on their side if necessary). Do not expose to excessive sunlight (air under dome can get hot).
8. Water and grow plants normally, tying up loose bolts with wax paper, tape, stakes, twist-ties, or other means. Stop watering as seeds become mature.
9. Harvest dry seed. Transformants are usually all independent, but are guaranteed to be independent if they come off of separate plants.
10. Select for transformants using antibiotic or herbicide selectable marker. For example, vapor-phase sterilize and plate 40 mg=2000 seed (resuspended in 4 ml 0.1% agarose) on 0.5×MS/0.8% tissue culture Agar plates with 50 ug/ml Kanamycin, cold treat for 2 days, and grow under continuous light (50-100 microEinsteins) for 7-10 days.
11. Transplant putative transformants to soil.
   For higher rates of transformation, plants may be dipped two or three times at seven day intervals. For example, dip two days after clipping, and dip again one week later. Do not dip less than 6 days apart.

### Example 3

### Expressing Sucrose Isomerase in Maize Increases Seed Weight

Recombinant vectors TG ZM 75, 77, 78, 81, and 88 were transformed into maize to generate T1 seeds. Table 3 below summarizes the results of a test evaluating the effect on average seed weight, one of the primary determinants of crop grain yield.

T1 seeds collected from transgenic plants selfed in the greenhouse, or sib pollinated between plants of related clones within a transgenic event (i.e., 3.3% of the total clones evaluated were sibs), were dried (3-4 days at approx. 32° C.) on the ear and shelled to collect grain. While processing ears from TG75, the inventors noticed that kernels on some ears appeared to be larger than typical compared to plants of other constructs. Subsequently, T1 seeds from all ears of TG_ZM 75 and several other constructs were measured for average kernel weight (air-dry basis). This trait was determined for each plant by weighing all seed from the ear, counting the number of kernels included in the weight, and taking the quotient as “average kernel weight”. Seed count to calculate the average kernel weight averaged 87 seeds. Mean kernel weight for each event was calculated by using the “average kernel weight” value of individual plants (i.e., clones) within an event and calculating an average trait value across those plants. When considering all events of constructs described herein, there was an average of 2.5 clones per event. To assess gene effects on the basis of individual constructs, the mean phenotype of all events within each construct was calculated.

The mean kernel weights from transgenic plants of TG_ZM 75, 77, 78, 81, and 88, all of which express a form of sucrose isomerase, were compared to mean kernel weights from transgenic plants of unrelated constructs (TG_ZM 74, 84, 85, 86, and 87, which comprise non-sucrose isomerase genes). The table below demonstrates that constructs TG_ZM 75, 77, 78 and 81 significantly increased seed weight by roughly 16% compared to the other constructs tested. The number of kernels per ear varied between individual plants, since these were grown in the greenhouse and hand-pollinated, but it did not appear that enhanced seed weights in select SI-containing constructs were related to differences in the number of seed produced on ears.

The average kernel weight (air-dry basis) of F2 seed from untransformed Hi-II maize plants has been determined to typically be approximately 180 mg per kernel. In comparison, the seed weight and/or size of kernels derived from selfed Hi-II plants transformed with non-sucrose isomerase genes in constructs TG_ZM 74, 84, 85, 86, and 87 approximates the same value (Table 3). This fact indicates that the non-sucrose isomerase genes transformed into maize and tested alongside the sucrose isomerase constructs do not likely reduce seed growth and average kernel weight. By contrast, the comparison between transgenic plants expressing sucrose isomerases and transgenic plants expressing non-sucrose isomerase genes indicates that expressing sucrose isomerase in maize can, in many cases, increase seed weight.

TABLE 3
Mean of Kernel Weights Across Events for Sucrose Isomerase and non-Sucrose Isomerase Constructs

| | | Total # | Mean of Kernel | Std. Error of Kernel |
|---|---|---|---|---|
| | # Events | Plants | Weights Across | Weights Across |
| Construct | Tested | Tested | Events (mg/kernel) | Events (mg/kernel) |
| TG_ZM 74 (ENOD40::non-SI 1) | 12 | 34 | 175 | 6 |
| TG_ZM 75 (ENOD40::SI Pd UQ68J) | 26 | 49 | 216 | 9 |
| TG_ZM 77 (EMOD40::SI Pd moUQ68J) | 16 | 44 | 218 | 14 |
| TG_ZM 78 (OMT::SI Pm MutB) | 20 | 52 | 212 | 8 |
| TG_ZM 81 (ENOD40::SI Pm MutB) | 5 | 11 | 211 | 5 |
| TG_ZM 84 (Actin::non-SI 2) | 15 | 31 | 182 | 8 |
| TG_ZM 85 (OMT::non-SI 2) | 10 | 20 | 191 | 12 |
| TG_ZM 86 (ENOD40::non-SI 2) | 18 | 44 | 186 | 8 |
| TG_ZM 87 (JAS::non-SI 2) | 18 | 59 | 177 | 7 |
| TG_ZM 88 (JAS::SI Pd UQ68J) | 16 | 46 | 187 | 6 |

SI: Sucrose Isomerase
non-SI 1: A gene that produces a hemicellulose hydrolytic enzyme, which is not a sucrose isomerase gene
non-SI 2: A gene that produces an enzyme capable of modifying the chemical character of various polysaccharides, which is not a sucrose isomerase gene

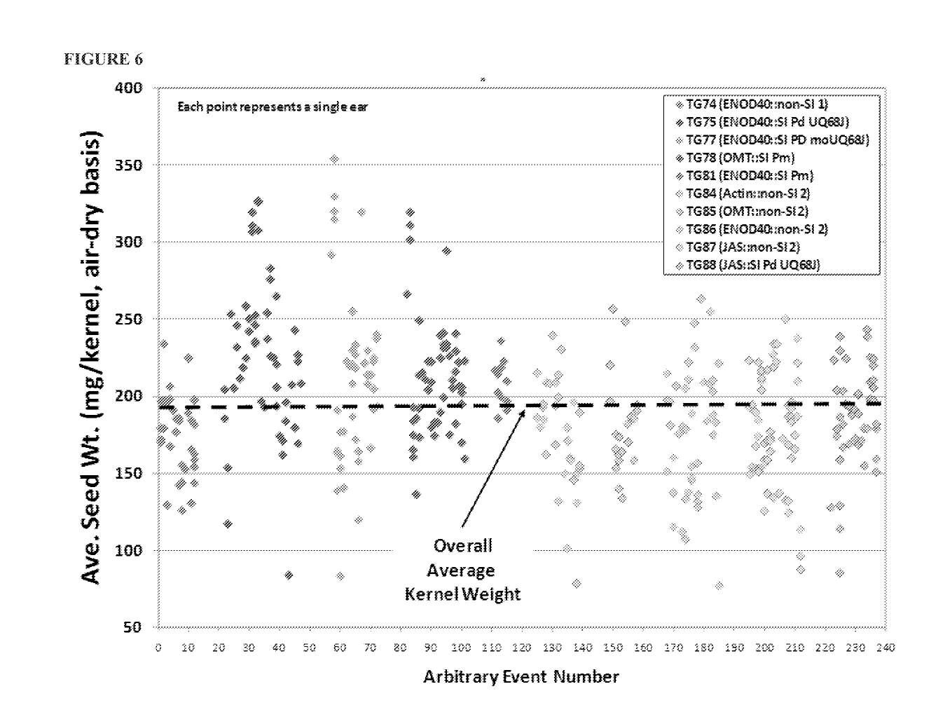

The effect of sucrose isomerase (SI) on kernel weight of each individual transgenic line is shown graphically in FIG. 6 where data for each individual ear of 10 constructs is displayed. Points aligned vertically are from clones (plants) of a single event.

The average kernel weight across all samples is 194 mg and the seed weight for most constructs ranged from approximately 125-250 mg. A range of this magnitude for kernel weight is not unusual for untransformed Hi-II plants since the parents of Hi-II are not fully inbred and transformation utilized F2 embryos. The range of kernel weights for TG_ZM 75, 77 and 78 extends well above that of other constructs for most, but not all, events. This indicates that the sucrose isomerase gene was efficacious in producing heavier seed for most events, but other events among these constructs had kernel weights typical of that occurring in the non-sucrose isomerase constructs. Although fewer events were tested with TG_ZM 81, the sucrose isomerase gene raised the mean kernel weight to the same level as seen for TG_ZM 75, 77, and 78.

It is noteworthy that three different forms of sucrose isomerases, using two different stalk preferred promoters, were effective in producing seed with greater average weight. With the ENOD40 promoter, which is specific to expression in cells straddling the vasculature of stems (rice), sucrose isomerase genes from both Pantoea dispersa UQ68J and Pseudonmonas mesoacidophila MX-45 favorably affected seed growth. Also, the o-methyl transferase (OMT) promoter was equally active in causing plants to make heavier seed when the SI from Pseudomonas mesoacidophila MX-45 was used. It appears from the effectiveness of the ENOD40 and OMT promoters that tissue specific expression is a key factor in allowing sucrose isomerases to affect assimilate partitioning and seed growth. However, the Pantoea dispersa UQ68J sucrose isomerase gene driven by the JAS promoter did not lead to plants with greater kernel weights. The JAS promoter has been reported to express in stem cells of sugarcane, but specific information relating to its strength or spatial and/or temporal specificity in maize has not been reported. It is possible that expression of sucrose isomerases with JAS failed to increase seed weight in maize because this promoter expresses weakly or otherwise inappropriately for the methods of this invention.

Sucrose isomerases produced from Pantoea dispersa UQ68J and Pseudomonas mesoacidophila MX-45 MutB genes are known to have widely different kinetic properties (Wu and Birch, 2005). Sucrose isomerase from UQ68J produces primarily isomaltulose while the MX-45 MutB protein synthesizes primarily trehalulose. Despite this, and other biochemical differences in the properties of these proteins, both utilize sucrose as a substrate and surprisingly can produce seeds with increased average weight when allowed to act on the pool of sucrose in stem tissues.

### Example 4

### Further Confirming Sucrose Isomerase Effects and Introducing Sucrose Isomerase into Commercial Maize Line to Increase Seed Weight

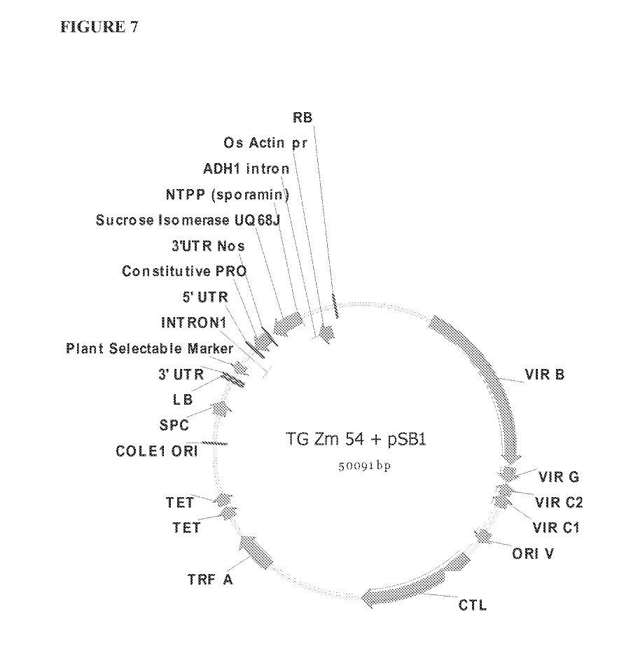

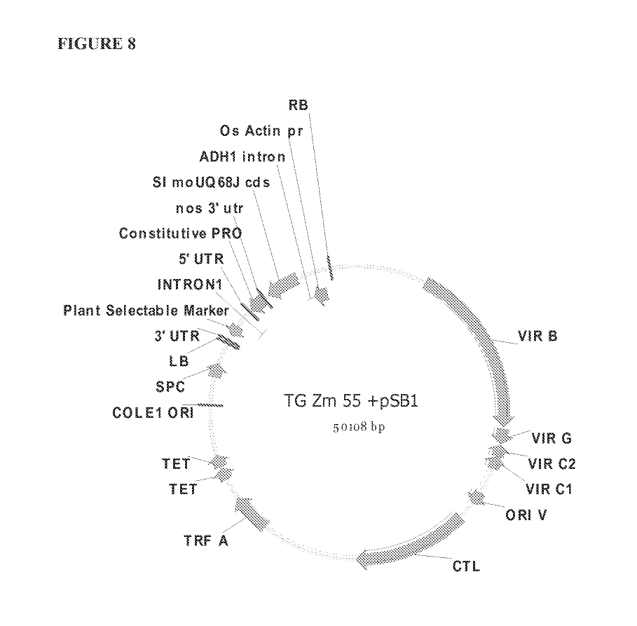

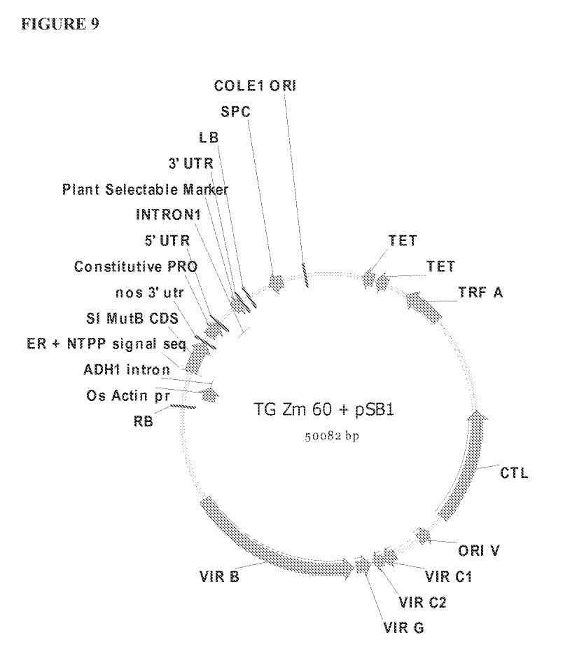

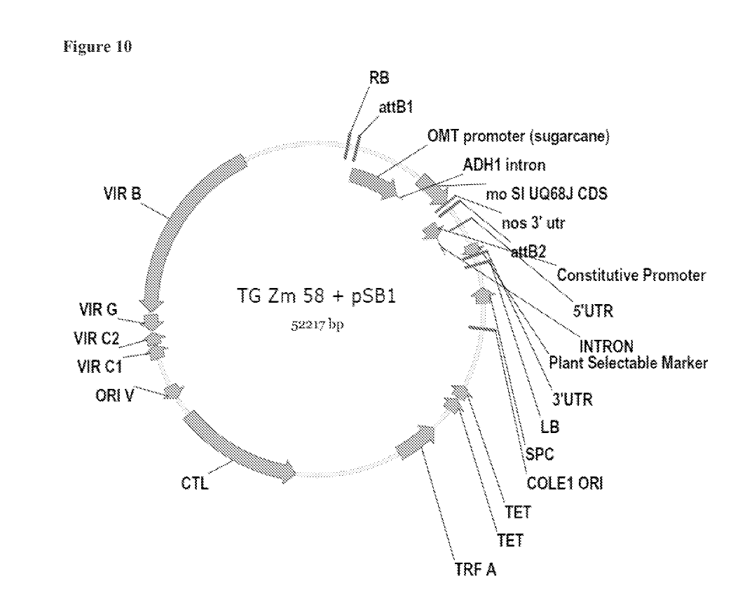

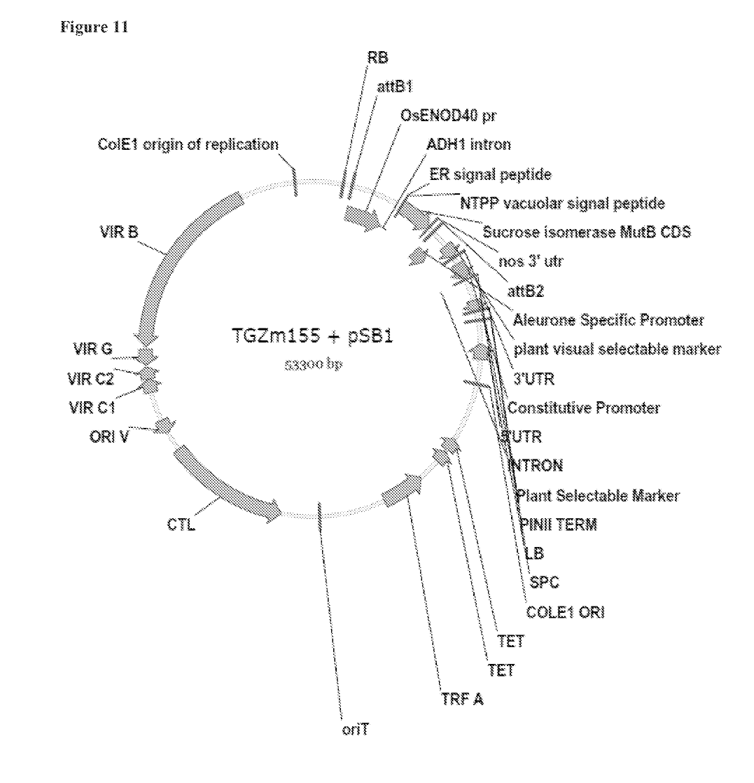

The inventors currently have at least 13 constructs (Tables 2 and 4) made and transformed into maize for evaluation of a sucrose isomerase effect. Distributed across these is the use of five different promoters, two of which to date have proven to be effective with sucrose isomerases in producing larger seed. T1 seed has been produced and evaluated for kernel weight from at least eight of the 13 sucrose isomerase constructs. Sucrose isomerase events carrying constructs TG_Zm #54, TG_Zm #55, TG_Zm #60, TG_Zm #58 and TG_Zm #155 (FIGS. 7, 8, 9, 10 and 11) will be evaluated in the field to determine the effects of sucrose isomerase on seed weight and/or size.

TABLE 4
Additional Sucrose Isomerase Recombinant
Vectors for Maize Transformation

| | Pro- | In- | Gene + | |
|---|---|---|---|---|
| TG_Zm # | moter | tron | 3′UTR | Gene source |
| TG_Zm #49 | OsMAD6 | ADH1 | SI Pd | Pantoea dispersa |
| | pr | | moUQ68J | UQ68J |
| TG_Zm #50 | JAS pr | ADH1 | SI Pd | Pantoea dispersa |
| | | | moUQ68J | UQ68J |
| TG_Zm #54 | Actin pr | ADH1 | SI Pd | Pantoea dispersa |
| | | | UQ68J | UQ68J |
| TG_Zm #55 | Actin pr | ADH1 | SI Pd | Pantoea dispersa |
| | | | moUQ68J | UQ68J |
| TG_Zm #58 | OMT pr | ADH1 | SI Pd | Pantoea dispersa |
| | | | moUQ68J | UQ68J |
| TG_Zm #60 | Actin | ADH1 | SI Pm | Pseudomonas |
| | | | MutB | mesoacidophila |
| | | | | MX-45 |
| TG_Zm #89 | OMT | ADH1 | SI Pd | Pantoea dispersa |
| | | | UQ68J | UQ68J |
| TG_Zm #155* | ENOD40 | ADH1 | SI Pm | Pseudomonas |
| | | | MutB | mesoacidophila |
| | | | | MX-45 |

*TG_Zm #155 differs from TG_Zm #81 in that an additional visual selectable marker is included in the expression vector.

Kernel weight increases were detected with constructs TG_ZM 75, 77, 78 and 81. T1 seed from large-kernel events of these four constructs was planted in the greenhouse for selfing plants and crossing them onto commercial maize inbred line.

### Isolated Crossing Block (ICB)

The overall objective of the isolated crossing block trials is to assess the influence of the genes being tested on productivity of grain per plant and the two underlying yield components, kernel number per ear and average kernel dry weight.

In most cases, seed being planted are F1 hybrid seed. They are produced by crossing one type of plant (A), which carries the gene of interest and typically contains 50-75% elite corn breeding germplasm background (i.e., recurring parent), with a second type (B) that is a commercial elite inbred of a counter heterotic group. In other cases, seed being planted are BC1 or BC2, meaning that they contain, on average, 75 or 87% elite corn breeding germplasm background (i.e., recurring parent).

Some events in the ICB trials employ a visual color marker gene which helps segregate Null seed (yellow, lacks transgene) from Transgenic seed (reddish, contains transgene). Plants of events which do not contain the color marker are identified and tagged as Null or Transgenic by applying (i.e., leaf painting) a small band of glufosinate herbicide to a narrow region of a leaf and seven days later scoring the plants for dead leaf tissue (Null) or green herbicide-resistant leaf tissue (Transgenic).

In the ICB design, rows are planted with a mix of Null and Transgenic seed, so plants of these two types occur at random positions within each row. These are considered female rows. Female plots consist of four female rows, each 17.5 ft. long with rows 30 inches apart. Planted adjacent to each female plot are two male rows, which are planted with the recurring parent referred to above.

At all ICB trial locations, plants in female rows will be pollinated by pollen released from the recurring parent planted in the male rows. Therefore, each ICB field will be planted in a pattern of two male rows for every four female rows.

Following plant maturity and dry down, ears will be harvested from plants within each female row and segregated as originating from a Null or Transgenic plant. This is done on the basis of whether ears contain color marked seed (Transgenic), or not (Null), or on the basis of the leaf paint results. Individual ears will be shelled and the grain weighed and measured for moisture level. A grain subsample will be collected from each ear for determination of 100-kernel weight, which in turn allows estimation of the number of kernels on each ear. Thus, we will determine grain productivity per ear and the core kernel weight and kernel number yield components.

### Measurements of Sucrose Isomerase “Inbred” Plants Grown in Isolated Crossing Block

Sucrose isomerase “inbred” plants grown in isolated crossing blocks are measured, for the purpose of confirming/refuting the sucrose isomerases' effect on seed weight and/or size, and also for providing insight to mechanism of action. The measurements include:

a. Plant height (minimum, V8 and R1), i.e., about thigh-high and at pollination
b. Stalk diameter (minimum, V8, R1, R6), i.e., thigh-high, pollination, maturity
c. Leaf sample for sugar, starch (maybe isomaltulose) determination, maybe also specific leaf wt. (at least at V8). Defined numbers of leaf punches collected from several leaves on plant is used. Analytically, not many punches are needed at all. This test indicates if carbohydrates metabolism is grossly altered or not. Without wishing to be bound by certain theory, if kernel weight and yield are increased, more carbohydrates should have been synthesized. Time-coursed samples may be collected for this purpose, e.g., samples can be collected in light (˜4 PM) and in dark (5 AM). The leaf would be “fully” charged, or depleted, respectively, of starch (i.e., reserve, buffer C).
d. Flowering date. This is a little tricky because they require detasseling. One way to proceed is detasseling and open pollination for accurate “IE yield” estimate. Flowering can be gauged by time of tassel emergence or, more likely, silk emergence.
e. Plant lodging
f. Once kernels are physiologically mature, Individual Ears (IEs) are harvested for:

i. IE dry grain wt. (ear yield) . . . ave. kernel wt by calculation

ii. IE kernel number

iii. IE grain composition, by NIR, if average seed weight, seed number and/or seed size is affected.

Meanwhile, stalk is collected for stalk dry weight measurement. Stalks are kept matched to specific ears so, if ear yield increased, stalks are weighed to know if the stalk became significantly lighter (i.e., weaker) due to heavier seed. This may imply where the extra carbohydrates come from to make bigger seed. Since these plants are detasseled, de-leafed stalks from ground level to 3 nodes above ear node are collected for this purpose. After drying and weighing, specific internodes collected are subjected to determination of sugars, starch, isomaltulose, and trehalulose, et al., through GC or other methods.

### Sucrose Isomerase Constructs Give Increased Seed Yield

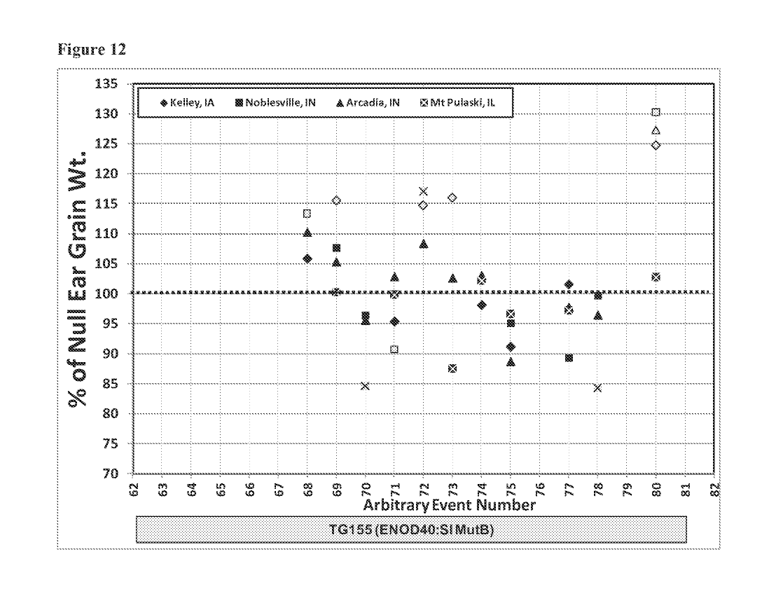

In ICB trials conducted across four locations, TG_ZM 155 (OsENOD40 promoter-Sucrose Isomerase Pm MutB) significantly increased ear grain weight, with five of eleven events having a significant increase in ear grain weight at least at one location compared to the Null sibling control. Arbitrary Event 80 in particular (155-A022), significantly increased yield (25-30%) in 3 out of 4 locations tested. See FIG. 12.

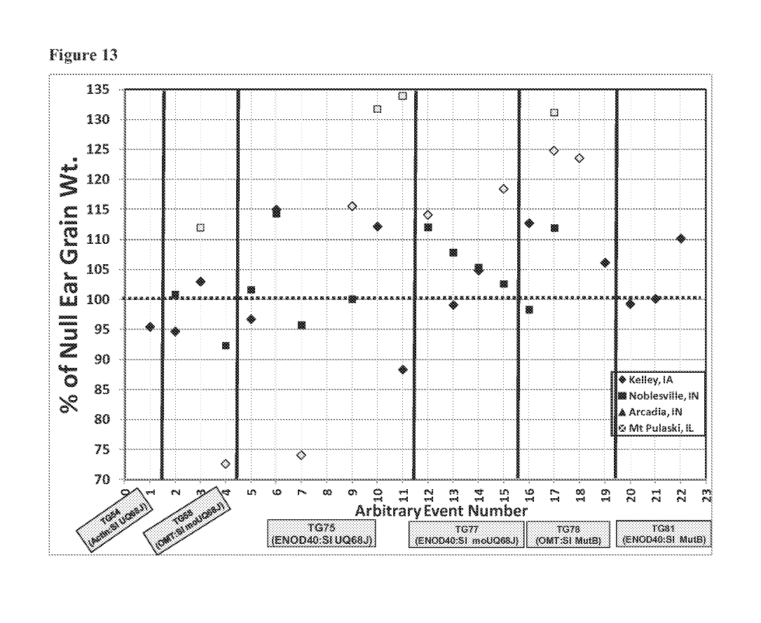

In ICB trials conducted across two locations. TG_Zm 58 (OMT promoter-Sucrose isomerase modified UQ68J), TG_Zm 75 (OsENOD40 promoter-Sucrose Isomerase UQ68J), TG_Zm 77 (OsENOD40 promoter-Sucrose Isomerase modified UQ68J) and TG_Zm 78 (OMT promoter-Sucrose isomerase Pm MutB) all had at least one event having a significant increase in ear grain weight at least at one location compared to the null sibling control. See FIG. 13.

### Example 5

### Expressing Sucrose Isomerase in Rice Increases Seed Weight and/or Size

To determine whether expression of sucrose isomerase in the vacuoles of stalk cells or of all cells in another monocotyledonous crop, rice (Oryza sativa var. indica or japonica), leads to increased average kernel weight and/or kernel size, constructs are made for Agrobacterium-mediated transformation into rice (Hiei and Komari, Nature Protocols, 3(5), 824, (2008)). A cassette containing a sucrose isomerase gene operably linked to a stalk specific promoter or a constitutive promoter and including an N-terminal ER sequence from sweet potato sporamin, an N-terminal propeptide (NTPP) sequence from sweet potato sporamin for targeting of the sucrose isomerase to the vacuole, and a suitable 3′ terminator is ligated into the binary vector pMH20 at the SmaI site. The stalk specific promoter can be, but is not limited to, the rice stem specific promoter ENOD40, the o-methyl transferase promoter OMT, or the jasmonate-inducible protein promoter JAS. The constitutive promoter can be, but is not limited to, the rice actin promoter (SEQ ID NO. 28 herein, as also described in U.S. Pat. No. 6,429,357). Additionally, a tissue specific promoter such as OsMAD6 (US 2006/0260011 A1), which expresses in inflorescences, ovary and early embryo of rice (The Bio-Array Resource for Plant Functional Genomics, University of Toronto), can be used. The sucrose isomerase gene can be the Pseudomonas mesoacidophila MX-45 (MutB), the Pantoea dispersa UQ68J, or a modified version of UQ68J (moUQ68J). The sucrose isomerase genes are codon optimized for rice.

Agrobacterium host EHA 105 carrying the resulting binary vector containing a sucrose isomerase cassette is selected by kanamycin antibiotic. The binary clones are restriction digested and sequenced to verify that the cassettes have been cloned at the appropriate sites and that the junctions are intact. Each binary vector containing a sucrose isomerase cassette or an empty binary vector (to serve as control) is transformed into rice calli by Agrobacterium-mediated transformation and transgenic calli is selected on hygromycin. Shoot and root formation of the transformed calli is induced by hormones and the resulting T0 plantlets are grown up in the greenhouse. T0 tissue is collected and Southerns done to confirm presence of the sucrose isomerase transgene and to determine transgene insert number. Typically single insert events are taken forward. About 15-30 T0 events will be obtained for each sucrose isomerase construct.

Total non-structural carbohydrates are extracted from T0 transgenic sucrose isomerase rice tissue (Heberer et al., Crop Science, 25(6), 1117 (1985)) and detected by HPLC (Zhang et al., Appl Environ Microbiol, 68(6), 2676 (2002)). Additionally, T1 mean kernel weight and/or size is measured. These preliminary analyses are compared to the corresponding measurements from transgenic events carrying the empty vector. It is expected that transgenic sucrose isomerase events have the non-plant sugars isomaltulose (using UQ68J or moUQ68J) or trehalulose (using MutB) compared to the empty vector events, and that T1 mean kernel weight and/or size is increased by 16% to 100% compared to the empty vector events. Sucrose isomerase enzyme activities may also be measured from T0 transgenic sucrose isomerase rice tissue extracts, although such measurements may be difficult if the enzyme degrades too quickly.

Reproducibility of the increased kernel weight and/or size is determined in subsequent generations and in newly created rice hybrids in the greenhouse and field and correlated with sugar type, sugar quantity, and sucrose isomerase enzyme expression.

### Example 6

### Expressing Sucrose Isomerase in Arabidopsis thaliana Increases Seed Weight and/or Size

To determine whether expression of sucrose isomerase in the vacuoles of stem cells or of all cells in the dicotyledonous model organism, Arabidopsis thaliana, leads to increased average seed weight, seed number and/or seed size, constructs are made for Agrobacterium-mediated transformation into Arabidopsis. A cassette containing a sucrose isomerase gene operably linked to a stem specific or stem preferred promoter or a constitutive promoter and including an N-terminal ER sequence from sweet potato sporamin, an N-terminal propeptide (NTPP) sequence from sweet potato sporamin for targeting of the sucrose isomerase to the vacuole, and a suitable 3′ terminator is ligated into a suitable binary vector. The stem specific or stem preferred promoter can be, but is not limited to, AtCesA4, AtCesA7 and AtCesA8 (promoters of cellulose synthase genes, US 2005/0086712 A1, WO 2000/070058, Taylor et al., Plant Cell, 11(5), 769 (1999), Taylor et al., Plant Cell, 12, 2529 (2000), Jones et al., Plant Journal, 26(2), 205 (2001)). The constitutive promoter can be, but is not limited to, the cauliflower mosaic virus CaMV 19S and 35S promoters (U.S. Pat. Nos. 5,352,605; 5,530,196 and 5,858,742 for example). The sucrose isomerase gene can be the Pseudomonas mesoacidophila MX-45 (MutB), the Pantoea dispersa UQ68J, or a modified version of UQ68J (moUQ68J). The sucrose isomerase genes are codon optimized for Arabidopsis.

Agrobacterium host At503 carrying the resulting binary vector containing a sucrose isomerase cassette is selected by appropriate antibiotics. The binary clones are restriction digested and sequenced to verify that the cassettes have been cloned at the appropriate sites and that the junctions are intact. Each binary vector containing a sucrose isomerase cassette or an empty binary vector (to serve as control) is transformed into Arabidopsis by the floral dip method (Bent, Methods Mol Biol, 343, 87 (2006)) and T1 seeds are selected on appropriate selection. Putative transformed T1 seeds are collected and sown in the greenhouse to get T1 plants. T1 tissue is collected and real-time PCR or Southerns done to confirm presence of the sucrose isomerase transgene. Insert number is determined by segregation analysis of T2 seeds.

Total non-structural carbohydrates are extracted from T1 transgenic sucrose isomerase Arabidopsis tissue (Heberer et al., Crop Science, 25(6), 1117 (1985)) and detected by HPLC (Zhang et al., Appl Environ Microbiol, 68(6), 2676 (2002)). Additionally, homozygous T2 mean seed weight and/or size is measured and compared to null siblings. It is expected that homozygous T2 mean seed weight and/or size is increased by 16% to 100% compared to the corresponding null siblings. Sucrose isomerase enzyme activities may also be measured from T1 transgenic sucrose isomerase Arabidopsis tissue extracts.

Reproducibility of the increased kernel weight and/or size would be determined in subsequent generations in the greenhouse and correlated with sugar type, sugar quantity, and sucrose isomerase enzyme expression, although measurements of the latter may be difficult if the enzyme degrades too quickly.

### Example 7

### Expressing Sucrose Isomerase in Camelina Increases Seed Weight and/or Size

To determine whether expression of sucrose isomerase in the vacuoles of stem cells or of all cells in a dicotyledonous crop, Camelina sativa, will lead to increased average seed weight, seed number and/or seed size, constructs are made for Agrobacterium-mediated transformation into Camelina. A cassette containing a sucrose isomerase gene operably linked to a stem specific or stem preferred promoter or a constitutive promoter and including an N-terminal ER sequence from sweet potato sporamin, an N-terminal propeptide (NTPP) sequence from sweet potato sporamin for targeting of the sucrose isomerase to the vacuole, and a suitable 3′ terminator is ligated into a suitable binary vector. The stem specific or stem preferred promoter can be, but is not limited to, AtCesA4, AtCesA7 and AtCesA8 (promoters of cellulose synthase genes, US 2005/0086712 A1, WO 2000/070058, Taylor et al., Plant Cell, 11(5), 769 (1999), Taylor et al., Plant Cell, 12, 2529 (2000), Jones et al., Plant Journal, 26(2), 205 (2001)). The constitutive promoter can be, but is not limited to, the cauliflower mosaic virus CaMV 19S and 35S promoters (U.S. Pat. Nos. 5,352,605; 5,530,196 and 5,858,742 for example). The sucrose isomerase gene can be the Pseudomonas mesoacidophila MX-45 (MutB), the Pantoea dispersa UQ68J, or a modified version of UQ68J (moUQ68J). The sucrose isomerase genes are codon optimized for Camelina.

An Agrobacterium host (EHA105, At503, or GV3101 (pMP90)) carrying the resulting binary vector containing a sucrose isomerase cassette is selected by appropriate antibiotics. The binary clones are restriction digested and sequenced to verify that the cassettes have been cloned at the appropriate sites and that the junctions are intact. Each binary vector containing a sucrose isomerase cassette or an empty binary vector (to serve as control) is transformed into Camelina by the floral dip method (WO 2009/117555) and T1 seeds are selected on appropriate selection. Putative transformed T1 seeds are collected and sown in the greenhouse to get T1 plants. T1 tissue is collected and real-time PCR or Southerns done to confirm presence of the sucrose isomerase transgene and to determine transgene insert number. Typically single insert events are taken forward. About 15-30 T0 events are obtained for each sucrose isomerase construct.

Total non-structural carbohydrates are extracted from T1 transgenic sucrose isomerase Camelina tissue (Heberer et al., Crop Science, 25(6), 1117 (1985)) and detected by HPLC (Zhang et al., Appl Environ Microbiol, 68(6), 2676 (2002)). Additionally, homozygous T2 mean seed weight and/or size is measured and compared to null siblings. It is expected that transgenic sucrose isomerase events have the non-plant sugars isomaltulose (using UQ68J or moUQ68J) or trehalulose (using MutB) compared to the empty vector events, and that homozygous T2 mean seed weight and/or size is increased by 16% to 100% compared to the corresponding null siblings. Sucrose isomerase enzyme activities may also be measured from T1 transgenic sucrose isomerase Camelina tissue extracts, although such measurements may be difficult if the enzyme degrades too quickly.

Reproducibility of the increased kernel weight and/or size would be determined in subsequent generations in the greenhouse and field and correlated with sugar type, sugar quantity, and sucrose isomerase enzyme expression, although measurements of the latter may be difficult if the enzyme degrades too quickly.

### Example 8

### Measurement of Sucrose Isomerase (SI) Enzyme Activity in Transgenic Callus Tissue

Expression and activity of SI in plant cells is monitored in transgenic callus tissue. Callus tissue is transformed with Agrobacterium containing empty vector or Agrobacterium containing the vacuole-directed SI under control of a constitutive promoter. Alternatively, transgenic SI plants that have been previously generated is used to generate callus material for analysis of sucrose isomerase function.

Callus material is used to monitor isomerase activity by several methods. One approach is to purify enough sucrose isomerase from callus material followed by in vitro isomerase assays. Alternatively, the callus material is grown on media containing carbon sources (see example 2) such as sucrose. The media is modified with different amounts of sucrose to optimize conversion of sucrose into isomaltulose and/or trehalulose. Transgenic calli are harvested and total non-structural carbohydrates are isolated and prepared for HPLC analysis. Sucrose isomerase expression at the transcript level as well at the protein level is also monitored in callus.

### Example 9

### Measurement of Corn Kernel Size in Transgenic Sugar Isomerase Events

Seed volume of kernels from all sugar isomerase events and their null sibling events are measured. A gas pycnometer with helium is used to determine volumes of corn kernel samples from sugar isomerase and null events. Seed volume is determined either with a wax coating to fill in any void volumes or without wax coating (Chang C. S. Cereal Chem 65(1):13-15 (1988)). The results can indicate that kernels from transgenic plants with sugar isomerase have obvious bigger size than the kernels from their null siblings. The kernel size of transgenic plants expressing sucrose isomerases can increase at least 1%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 105%, at least 110%, at least 120%, at least 130%, at least 140%, at least 150%, at least 160%, at least 170%, at least 180%, at least 190%, at least 200%, or more compared to the controls (e.g, kernels of null siblings).

### Example 10

### Expressing Sucrose Isomerase in Cereals Increases Grain Test Weight

Differences in apparent density between grain samples can be associated with the size, arrangement, and efficiency of “packing” of cells in the endosperm, the primary tissue of the kernel. They can also be a function of diversity in the form, size, and molecular composition of starch granules which comprise the majority of material in endosperm cells (Lu et al. (1996) Carbohydrate Res 282:157-170).

Expression of sucrose isomerase genes in plants can modify assimilate partitioning, metabolite utilization, and growth within the plant and lead to formation of seed which are heavier than analogous control seed of nontransformed plants. The increase in seed weight is not coincident with a directly proportional increase in volume of intact kernels due to an increase in apparent density of the seed. As a result, sucrose isomerase genes can be used to influence mechanisms within developing seed that determine the “compactness” of growth and how efficiently cells and their contents are ordered in space to minimize intracellular and intercellular areas lacking tangible matter and maximizing the accumulation of cellular substances which add mass to the seed and its subunit organs.

Because sucrose isomerase genes can increase grain apparent density, they also increase grain test weight, improve grain quality, and assist crop producers in maximizing financial compensation for their grain crop. The effect of these genes on seed growth and development improve, and/or sustain, the quality of the grain by influencing test weight. Although the “weight” bushel is used as the basis of payment for grain, price discounts are often coupled to consignments of lower grade grain that possesses low test weight. Expression of sucrose isomerase in cereal plants can be utilized to increase, or preserve, grain test weight and grade designations that bring higher payment.

Unless defined otherwise, all technical and scientific terms herein have the same meaning as commonly understood by one of ordinary skill in the art to which this invention belongs. Although any methods and materials, similar or equivalent to those described herein, can be used in the practice or testing of the present invention, the preferred methods and materials are described herein. All publications, patents, and patent publications cited are incorporated by reference herein in their entirety for all purposes.

The publications discussed herein are provided solely for their disclosure prior to the filing date of the present application. Nothing herein is to be construed as an admission that the present invention is not entitled to antedate such publication by virtue of prior invention.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

### REFERENCES

1. Veronese T, Perlot P. “Proposition for the biochemical mechanism occurring in the sucrose isomerase active site.” FEBS Lett. 1998 Dec. 28; 441(3):348-52.
2. Börnke F, Hajirezaei M, Sonnewald U. “Cloning and characterization of the gene cluster for palatinose metabolism from the phytopathogenic bacterium Erwinia rhapontici.” J. Bacteriol. 2001 April; 183(8):2425-30.
3. Börnke F, Hajirezaei M, Heineke D, Melzer M, Herbers K, Sonnewald U., “High-level production of the non-cariogenic sucrose isomer palatinose in transgenic tobacco plants strongly impairs development.” Planta. 2002 January; 214(3):356-64.
4. Börnke F, Hajirezaei M, Sonnewald U. “Potato tubers as bioreactors for palatinose production.” J Biotechnol. 2002 Jun. 13; 96(1): 119-24.
5. Hajirezaei M R, Börnke F, Peisker M, Takahata Y, Lerchl J. Kirakosyan A, Sonnewald U., “Decreased sucrose content triggers starch breakdown and respiration in stored potato tubers (Solanum tuberosum).” J Exp Bot. 2003 January; 54(382):477-88.
6. Wu L, Birch R G., “Characterization of Pantoea dispersa UQ68J: producer of a highly efficient sucrose isomerase for isomaltulose biosynthesis.” J Appl Microbiol. 2004; 97(1):93-103.
7. Wu L, Birch R G., “Characterization of the highly efficient sucrose isomerase from Pantoea dispersa UQ68J and cloning of the sucrose isomerase gene.” Appl Environ Microbiol. 2005 March, 71(3):1581-90.
8. Ravaud S, Watzlawick H, Haser R, Mattes R, Aghajari N., “Expression, purification, crystallization and preliminary X-ray crystallographic studies of the trehalulose synthase MutB from Pseudomonas mesoacidophila MX-45.” Acta Crystallogr Sect F Struct Biol Cryst Commun. 2005 Jan. 1; 61 (Pt 1):100-3. Epub 2004 Dec. 2.
9. Ravaud S. Watzlawick H. Haser R, Mattes R, Aghajari N., “Overexpression, purification, crystallization and preliminary diffraction studies of the Protaminobacter rubrum sucrose isomerase SmuA.”, Acta Crystallogr Sect F Struct Biol Cryst Commun. 2006 Jan. 1; 62 (Pt 1):74-6. Epub 2005 Dec. 16.
10. Wu L, Birch R G., “Doubled sugar content in sugarcane plants modified to produce a sucrose isomer.” Plant Biotechnol J. 2007 January; 5(1):109-17.
11. Park S E, Cho M H, Lim J K, Kim J S, Kim J H, Kwon D Y, Park C S., “A new colorimetric method for determining the isomerization activity of sucrose isomerase.”, Biosci Biotechnol Biochem. 2007 February; 71(2):583-6. Epub 2007 Feb. 7.
12. Ravaud S, Robert X, Watzlawick H, Haser R, Mattes R, Aghajari N. “Trehalulose synthase native and carbohydrate complexed structures provide insights into sucrose isomerization.” J Biol. Chem. 2007 Sep. 21; 282(38):28126-36. Epub 2007 Jun. 27.
13. Lee H C, Kim J H, Kim S Y, Lee J K. “Isomaltose production by modification of the fructose-binding site on the basis of the predicted structure of sucrose isomerase from “Protaminobacter rubrum”.” Appl Environ Microbiol. 2008 August; 74(16):5183-94. Epub 2008 Jun. 13.
14. Ravaud S, Robert X, Watzlawick H, Haser R, Mattes R, Aghajari N. “Structural determinants of product specificity of sucrose isomerases.” FEBS Lett. 2009 Jun. 18; 583(12):1964-8. Epub 2009 May 8.
15. Cha J. Jung J H, Park S E, Cho M H, Seo D H, Ha S J. Yoon J W, Lee O H. Kim Y C, Park C S. “Molecular cloning and functional characterization of a sucrose isomerase (isomaltulose synthase) gene from Enterobacter sp. FMB-1.” J Appl Microbiol. 2009 October; 107(4): 1119-30. Epub 2009 Mar. 30.
16. Watzlawick H, Mattes R. “Gene cloning, protein characterization, and alteration of product selectivity for the trehalulose hydrolase and trehalulose synthase from “Pseudomonas mesoacidophila” MX-45.”, Appl Environ Microbiol. 2009 November; 75(22):7026-36. Epub 2009 Sep. 25.
17. Buchman, A. R., and Berg, P. (1988). Comparison of intron-dependent and intron-independent gene expression. Mol. Cell. Biol. 8: 4395-4405.
18. Palmiter, R. D., Sandgren, E. P., Avarbock, M. R., Allen, D. D., and Brinster, R. L. (1991). Heterologous introns can enhance expression of transgenes in mice. Proc. Natl. Acad. Sci. USA 88: 478-482
19. Okkema, P. G., Harrison, S. W., Plunger, V., Aryana, A., and Fire, A. (1993). Sequence requirements for myosin gene expression and regulation in Caenorhabditis elegans. Genetics 135: 385-404.
20. Koziel, M. G., Carozzi, N. B., and Desai, N. (1996). Optimizing expression of transgenes with an emphasis on post-transcriptional events. Plant Mol. Biol. 32: 393-405.
21. Simpson, G. G., and Filipowicz, W. (1996). Splicing of precursors to mRNA in higher plants: Mechanism, regulation and sub-nuclear organisation of the spliceosomal machinery. Plant Mol. Biol. 32: 1-41.
22. Duncker, B. P., Davies, P. L., and Walker, V. K. (1997). Introns boost transgene expression in Drosophila melanogaster. Mol. Gen. Genet. 254: 291-296
23. Lugones, L. G., Scholtmeijer, K., Klootwijk, R., and Wessels, J. G. (1999). Introns are necessary for mRNA accumulation in Schizophyllum commune. Mol. Microbiol. 32: 681-689
24. Ho, S. H., So, G. M., and Chow, K. L. (2001). Postembryonic expression of Caenorhabditis elegans mab-21 and its requirement in sensory ray differentiation. Dev. Dyn. 221: 422-430
25. Xu, J., and Gong, Z. Z. (2003). Intron requirement for AFP gene expression in Trichoderma viride. Microbiology. 149: 3093-3097.
26. Xu, Y., Yu, H., and Hall, T. C. (1994). Rice triosephosphate isomerase gene 5′ sequence directs β-glucuronidase activity in transgenic tobacco but requires an intron for expression in rice. Plant Physiol. 106: 459-467.
27. Chemy, J. R., Fidantsef, A. L., 2003. Directed evolution of industrial enzymes: an update. Current Opinion in Biotechnology 14, 438-443.
28. Sylvestre, J., Chautard, H., et al., 2006. Directed evolution of biocatalysts. Organic Process Research and Development 10:562-571.
29. Yun, C., Matsuda, H., et al., 2006. Directed evolution to enhance secretion efficiency and thermostability of chitosanase from mitsuaria chitosanitabida 3001. Bioscience Biotechnology and Biochemistry 70(2):559-563.
30. Chautard, H., Blas-Galindo. E., et al., 2007. Anactivity-independent selection system of thermostable protein variants. Nature Methods 4(11):919-921
31. Joyce, G. F., 1994. In vitro evolution of nucleicacids. Current Opinion in Structural Biology 4:331-336.
32. Piatesi, A., Howland, S. W., et al., 2006. Directed evolution for improved secretion of cancer-testis antigen NY-ESO-1 from yeast. Protein Expression and Purification 48:232-242.
33. Chang C S, “Measuring density and porosity of grain kernels using a gas pycnometer.” Cereal Chem. 1988; 65(1):13-15.
34. Lu T J, Jane J L, Keeling P L, Singletary G W. “Maize starch fine structures affected by ear developmental temperature.” Carbohydrate Res. 1996; (1)282:157-170.
35. Hasjim J, Srichuwong S, Scott M P, Jane J L. “Kernel composition, starch structure, and enzyme digestibility of opaque-2 maize and quality protein maize.” J. Agric. Food Chem. 200; 57:2049-2055.

1. An expression vector comprising a polynucleotide having a nucleic acid sequence encoding a sucrose isomerase, or biologically active variant, or fragment thereof, wherein the polynucleotide is operably-linked to a stem specific and/or stem preferred promoter.

2. (canceled)

3. The expression vector of claim 1, wherein the sucrose isomerase is encoded by a polynucleotide sequence selected from the group consisting of:
(i) SEQ ID NOs: 1, 3, 7, 9, 14, biologically active variants, and fragments thereof;
(ii) a sequence sharing at least 65% identity to a gene encoding the polypeptide of SEQ ID NO: 2, 4, 8, 10, 13, or 15 wherein the sequence encodes a functional sucrose isomerase, biologically active variants, and fragments thereof; and
(iii) a sequence that can hybridize under stringent condition with a nucleic acid sequence encoding the polypeptide of SEQ ID NO 2, 4, 8, 10, 13 or 15, or biologically active variants, or fragments thereof;
and optionally, wherein the polynucleotide sequence is codon-optimized for plant expression.

4. The expression vector of claim 1, wherein the stem specific and/or stem preferred promoter is a promoter associated with an early nodulin (ENOD) gene or an o-methyltransferase (OMT) gene, homologs thereof, or functional variants or fragments thereof.

5. (canceled)

6. The expression vector of claim 4, wherein the promoter is a rice ENOD40 promoter or a sugarcane o-methyl transferase (OMT) promoter, homologs thereof, or functional variants or fragments thereof.

7. The expression vector of claim 1, wherein the polynucleotide having a nucleic acid sequence encoding the sucrose isomerase is in-frame tagged with a subcellular localization signal peptide.

8. (canceled)

9. The expression vector of claim 7, wherein the subcellular localization signal peptide is an ER signal peptide, a vacuolar targeting signal peptide, or combination thereof.

10-11. (canceled)

12. The expression vector of claim 1, wherein a plant gene intron sequence is between the plant promoter and the polynucleotide encoding the sucrose isomerase, and wherein the intron sequence leads to intron-mediated enhancement (IME) of sucrose isomerase expression.

13. (canceled)

14. A method for increasing average seed weight and/or seed number in a plant comprising incorporating into the plant a transgene comprising a polynucleotide having a nucleic acid sequence encoding a sucrose isomerase, or biologically active variant, or fragment thereof.

15. The method of claim 14, wherein the polynucleotide is operably-linked to a stem specific and/or stem preferred promoter.

16-21. (canceled)

22. The method of claim 15, wherein the stem specific and/or stem preferred promoter is a promoter associated with an early nodulin (ENOD) gene or an o-methyltransferase (OMT) gene, homologs thereof, or functional variants or fragments thereof.

23-24. (canceled)

25. The method of claim 14, wherein the sucrose isomerase is encoded by a polynucleotide sequence selected from the group consisting of:
(i) SEQ ID NOs: 1, 3, 7, 9, 14, biologically active variants, and fragments thereof;
(ii) a sequence sharing at least 65% identity to a gene encoding the polypeptide of SEQ ID NO: 2, 4, 8, 10, 13, or 15 wherein the sequence encodes a functional sucrose isomerase, biologically active variants, and fragments thereof; and
(iii) a sequence that can hybridize under stringent condition with a nucleic acid sequence encoding the polypeptide of SEQ ID NO 2, 4, 8, 10, 13 or 15, or biologically active variants, or fragments thereof;
and optionally, wherein the polynucleotide sequence is codon-optimized for plant expression.

26. A transgenic plant expressing a sucrose isomerase, biologically active variants, or fragments thereof, wherein the transgenic plant has increased average seed weight and/or seed number compared to a control plant not expressing the sucrose isomerase, biologically active variants, or fragments thereof.

27. The transgenic plant of claim 26, wherein the sucrose isomerase is under the control of a stem specific and/or stem preferred promoter.

28. (canceled)

29. The transgenic plant of claim 26, wherein the plant is the dicotyledon plant selected from the group consisting of bean, soybean, peanut, nuts, members of the Brassicaceae family (Camelina, Canola, oilseed rape, etc.), cotton, peas, amaranth, tomatoes, sugarbeet, sunflower.

30. The transgenic plant of claim 26, wherein the plant is the monocotyledon plant selected from the group consisting of corn, rice, wheat, barley, sorghum, millets, oats, ryes, triticales, buckwheats, fonio, quinoa and oil palm.

31-33. (canceled)

34. The transgenic plant of claim 26, wherein the stem specific and/or stem preferred promoter is a promoter associated with an early nodulin (ENOD) gene or an o-methyltransferase (OMT) gene, homologs thereof, or functional variants or fragments thereof.

35-36. (canceled)

37. The transgenic plant of claim 26, wherein the sucrose isomerase is encoded by a polynucleotide sequence selected from the group consisting of:
(i) SEQ ID NOs: 1, 3, 7, 9, 14, biologically active variants, and fragments thereof;
(ii) a sequence sharing at least 65% identity to a gene encoding the polypeptide of SEQ ID NO: 2, 4, 8, 10, 13, or 15 wherein the sequence encodes a functional sucrose isomerase, biologically active variants, and fragments thereof; and
(iii) a sequence that can hybridize under stringent condition with a nucleic acid sequence encoding the polypeptide of SEQ ID NO 2, 4, 8, 10, 13 or 15, or biologically active variants, or fragments thereof;
and optionally, wherein the polynucleotide sequence is codon-optimized for plant expression.

38. A seed, a fruit, a pollen, or an ovule or a part of the transgenic plant of claim 26.

39-40. (canceled)

41. A genetically related plant population comprising the transgenic plant of claim 26.

42. A tissue culture of regenerable cells of the transgenic plant of claim 26.

43-45. (canceled)
